SEQUENCE LISTING (557 sequences)

SEQ ID NO: 1 - RNA, 411 nt, synthetic construct
  source 1..411: mol_type = other RNA
atgacagagtacaagctggtggtcgtgggggccaggggcgtgggcaagagcgccctgactatccagctggaatacaagctggtggtggtgggcgccgacggcgtggggaagtccgccctcacaatcgagtacaagctggtcgtggtgggggccgtgggcgtcgggaagtccgccctgacaatcgagtacaagttggtggtggtgggcgcctgcggggtgggcaagagcgccctgaccatctacaagctcgtggtggtgggggccggggacgtggggaagagtgccctgaccatccagtataagctggtggtggtcggcgccgggtgcgtgggcaagtccgccctgacaatccagctgctggatatcctggataccgccggccacgaggagtacagcgccatgagggatcag

SEQ ID NO: 2 - RNA, 357 nt, synthetic construct
  source 1..357: mol_type = other RNA
tacaagctggtcgtcgtgggggcctgcggcgtggggaagtccgctctgacctacaaactggtggtggtgggggccgatggggtgggcaagtccgccctgacctacaagctggtggtggtcggggccgtcggcgtgggcaaaagcgccctgacatacaagctcgtggtggtgggcgccaggggggtgggcaagtccgccctgacaaagctggtcgtggtgggcgccgggtgcgtgggcaagagcgccctgacaatcaagctggtggtggtgggggccggggatgtgggcaagagcgccctgaccatcctggacatcctggacacagccgggcacgaggagtacagcgccatgagggat

SEQ ID NO: 3 - RNA, 501 nt, synthetic construct
  source 1..501: mol_type = other RNA
ctgctgagcgtgggggggtacaagctcgtggtggtgggcgcctgcggcgtggggaagtccgccctgacactgctgtccgtgggcggctacaagctggtggtcgtgggcgccgacggggtgggcaagagcgccctcaccctgctcagcgtggggggctacaagctggtcgtggtcggggccgtgggcgtgggcaagtccgctctgacactgctgtccgtgggcgggtacaagctggtggtggtgggggccaggggggtcgggaaatccgccctgactctgctgagcgtcggcggcaagctcgtggtggtgggggccgggtgcgtggggaagtccgccctgacaattctgctgagcgtgggcgggaagctggtcgtggtgggcgccggggatgtgggcaagagcgccctgaccatcctgctgagcgtcggcggcctggacatcctggacaccgccggccacgaggagtacagcgccatgagggatctgctcagcgtggggggg

SEQ ID NO: 4 - RNA, 252 nt, synthetic construct
  source 1..252: mol_type = other RNA
gttggagcctgtggagttggtaaaagtctcctaagcgttggaggagtagttgtgggagcttgtggagtaggtctgttaagcgtgggtggtgtaggagcttgtggagtaggaaagtctctgctctccgtaggaggagtagtagtaggagcatgcggagtcggactgctgtccgtaggaggtgtaggagcatgcggagtaggaaagtcacttctgtctgtaggaggagtagtagtaggagcttgtggagtagga

SEQ ID NO: 5 - RNA, 252 nt, synthetic construct
  source 1..252: mol_type = other RNA
gtgggcgcctgtggagtaggaaaatctctcttatctgttggaggagtggtggtcggagcatgcggagtaggactcttgtccgtaggaggtgtgggagctgacggcgtgggtaaatccctattaagtgtcggaggagtggtcgtgggagctgacggtgttggtttattgagcgttggaggaggagcgggagatgttggaaagtcagccttacttagcgtcggaggagttgtgggggctggtgacgtgggaaag

SEQ ID NO: 6 - RNA, 441 nt, synthetic construct
  source 1..441: mol_type = other RNA
atgacagaatacaaacttgtggtagttggagcatgcggtgtgggtaagagtgccctcaccatccaactaattcagaaccacttcgttgacgaactcctctcagtggggggggttggagcatgtggtgtagggaagctgttgagcgtcggcgggatgactgaatacaaacttgtggttgttggagcagatggggtcggaaaatccgccttgacaatacagcttattcagaatcactttgtggatgagctgttaagcgttggcggcgtcggagctgatggcgtcggaaagctcttgtctgtgggcggcactgagtataaattagtggtagtcggggctggagacgtgggcaagagtgcccttaccatccagctcatccagaatcattttgtggacgagtatctattgtccgtgggaggtggagctggtgacgtcggaaagtct

SEQ ID NO: 7 - RNA, 648 nt, synthetic construct
  source 1..648: mol_type = other RNA
atgactgaatataaactcgtcgtggttggagcaggttgcgttggcaaaagcgcccttacaatccagctgatacagaaccacttcgtggatgagttattgagcgtcggcggcatgacagagtacaagttggtggtggtgggcgcctgtggggtggggaaatctgcccttactatccagctgatccagaaccactttgtggatgagttacttagcgtcggagggatgacagaatacaaactggtcgtggtcggcgctgatggcgttgggaagagtgctctgacaattcaactcatccagaatcacttcgtagacgaactgctttcagtcggggggatgacggagtataagctcgttgtggtaggcgccggagacgtaggcaagtccgcactgaccattcagctcattcaaaatcattttgtagacgaactattgtctgtgggcggtatgaccgagtacaagctggttgttgtgggtgccgtgggggtgggtaagtcagccctcaccattcagctcatccaaaaccatttcgtggacgagctgctgagtgtcggaggtacctgcctcctggacatattggacactgcggggcatgaggagtattccgcaatgcgcgatcagtacatgaggaccggagaaggatttctgtgc

SEQ ID NO: 8 - RNA, 351 nt, synthetic construct
  source 1..351: mol_type = other RNA
atgacagagtataagctggtcgtggtcggcgcgtgcggagtggggaaatcagctctcaccattcagctaatccagaaccacttcgttgacgaagtcggcgcttgcggtgtcggaaagatgacggagtataaacttgtcgtagttggcgccgacggtgttggaaaatccgcattaaccatacaattgatccagaaccacttcgtggacgaagtgggggctgacggcgtaggaaaaacagagtacaagctggtggtggttggcgcaggggatgtgggtaagagcgccctgactattcagctgatccaaaatcattttgtggatgaatacggagcaggcgatgtggggaagtct

SEQ ID NO: 9 - RNA, 1179 nt, synthetic construct
  source 1..1179: mol_type = other RNA
tgtataaacctcgattgggatgtaatccgcgacaaaaccaagacaaaaattgaatctctcaaggagcacggccctatcaaaaataaaatgtcagagtcacccaataagacggtgtctgaggaaaaggccaagcaatacttggaagaatttcatcaaaccgccttggaacatccggaactaagcgagcttaagacagttacaggtactaacccagtgtttgccggagctaactacgccgcgtgggcggtcaatgtagcccaggtcattgatagtgaaactgctgacaatttagaaaagacaactgccgctctctccattctgcccggaatagggagcgttatgggtatcgcagatggcgcggtgcatcataacaccgaggagatcgtcgcacagtcgatcgctctcagtagcctaatggtggctcaggcaattcctctggtcggggaactggttgacatcggattcgccgcatacaatttcgtagagtctattataaatcttttccaggtggtccacaactcatataatcgaatgactgagtacaagctggtagtggtaggggctggatgcgtcgggaagtcagccctgactatacagctgatccagaaccacttcgtggatgagctcctatccgtgggcgggatgacggaatataagctcgttgtggtaggggcttgtggagtgggcaagtccgcactgacaatccagctgatccagaatcactttgtggatgagctcctgagtgtggggggaatgaccgaatacaaacttgttgtggtgggcgctgacggagttggtaaaagcgcccttaccatccaactgattcagaaccactttgtcgacgagttactgagcgtcggagggatgaccgagtataaattggtggtggtgggcgctggcgacgtgggcaaaagtgcactgacaattcagctgattcagaaccactttgtggacgagttgctaagcgtcggtggaatgaccgagtataagttggtggttgtcggcgcggttggtgtgggaaaatctgcccttacaatccagctgattcaaaaccatttcgtcgacgagctgctgtccgttgggggcacgtgcttgctcgacatcctggatactgccggccacgaagagtactccgccatgcgggatcaatatatgcgtaccggcgaggggttcttatgt

SEQ ID NO: 10 - RNA, 1197 nt, synthetic construct
  source 1..1197: mol_type = other RNA
atgacagagtacaagctggtggtcgtcggagccggttgcgtaggaaagagcgcattaaccatccagctaatccaaaaccatttcgtggacgaactcctgtcagtggggggaatgactgaatacaagttagtggtggttggggcctgtggtgtgggaaaatccgctctcacgatccagctgattcagaaccacttcgtcgacgagttactttccgttggaggcatgaccgaatataaactggttgtcgtgggggctgatggagttggcaagtctgccttgactattcaactgatccagaatcattttgtggatgagctcctgagcgtaggggggatgacagagtataaactcgtcgtggtgggtgccggagatgttgggaaatctgctcttaccatacagttaatccaaaatcacttcgttgatgaactgctttcagtgggcggaatgactgagtacaagttggtggtggtgggggctgtgggggtgggaaagtctgcactgactattcagctcattcagaaccacttcgtggatgagctgctctcggtaggcgggacctgtctgctagacattctggacacggccggccatgaggagtatagcgcaatgagagaccaatacatgcggaccggcgaaggctttctttgcctgctcagcgtgggcggctgtataaacctcgactgggatgtgatccgcgataaaaccaagaccaagatcgaaagcctaaaggagcatggccctattaagaataaaatgtcagaatctcccaataaaacagtatccgaggaaaaggccaaacagtatttggaagagtttcaccagaccgccctggaacacccagagctgtccgaactgaagacagtcactgggacaaatcctgtcttcgccggtgctaactacgcggcatgggcggtcaatgtagcccaagttatcgatagtgagactgccgacaaccttgagaaaaccacagcagcattgagcatattgccgggcatcggttctgtcatgggcatagctgacggcgctgtgcaccacaacacagaggagatcgtcgctcagagtattgcgctgtccagtcttatggtggcccaggccatcccacttgttggtgaactggtggacatcggatttgcagcgtacaactttgtcgagtcaattattaatctgttccaggttgtccataacagttataatcgt

SEQ ID NO: 11 - RNA, 1155 nt, synthetic construct
  source 1..1155: mol_type = other RNA
tgtataaatcttgactgggacgtcattagggataagacaaagaccaagatagagtccttgaaggagcacggccccatcaaaaacaaaatgtcggagagcccaaacaaaacagtgagtgaagagaaagcaaagcagtatcttgaagagtttcaccagacggctctggaacatcctgagctgtctgaacttaagacagtgaccggcacaaaccctgtgtttgcaggagctaattatgcggcttgggccgttaacgtggctcagatgactgagtataaactggttgtggtgggcgccggctgcgtcggaaagagcgcgctgaccattcaacttattcaaaatcacttcgttgatgaactcctgtcagtaggaggcatgactgaatataagttagtggtcgtgggcgcctgtggggtgggtaaaagcgccctaactatacagctaatccagaaccactttgtagacgagcttctgtccgtggggggtatgacagagtacaagctggtggttgtaggtgcagacggcgtcggcaaatccgccctcactatccagctgattcagaaccactttgtagacgagttgctctcagtcggaggaatgactgaatacaaacttgttgtggttggggctggggatgtcggaaagagtgctctgacgattcagctgatccagaaccattttgtggacgaactcctgtcagttgggggcatgaccgagtacaagctggtggttgtgggagcggtcggggtcgggaagtccgccttaacaatccagctgatccaaaatcacttcgtggatgaactgttgtcagtggggggcacctgcctgctcgacatcctcgataccgccggtcacgaagagtactccgccatgcgtgatcaatatatgcgaacgggggagggtttcttatgcaatttggagaaaaccaccgcagcactatctatcctgccgggaataggtagcgtcatgggcatcgccgacggagcagtacatcataatactgaagaaattgtggcccaatctatcgccttaagttctttgatggtggctcaggctattcccctggtcggggagctggtagatattggcttcgctgcctacaacttcgtggagagcatcattaatttgttccaggtcgtgcataatagctacaacagg

SEQ ID NO: 12 - RNA, 408 nt, synthetic construct
  source 1..408: mol_type = other RNA
tacaagctagtggtggtcggcgcatgcggcgtgggaaagagcgcgctaacctataaattggtcgttgtgggcgccgatggtgtagggaaatctgcgctgacctacaagctcgtggtggtgggggcagtcggcgtcggcaaaagtgccctgacttataagctggtcgtggtaggtgctcgtggcgttggaaagtccgccttgacaaagctggttgtcgtgggggccggatgcgtgggcaaatctgctcttacaatcaaattagtagttgtgggagctggagacgtaggtaaatccgcactgacgatactcgatattctcgacaccgctggacacgaagagtacagcgccatgcgcgacaagctggtcgtggtgggggccggcagagttggtaagtcagcactcactatc

SEQ ID NO: 13 - RNA, 1206 nt, synthetic construct
  source 1..1206: mol_type = other RNA
atgaccgagtacaaactcgttgtggtgggggcctgcggagtgggcaagtctgcattgaccatacagttgatccagaatcatttcgtggatgagctcctaagtgtcggcggcgtcggagcgtgtggcgtgggcaaactcctcagcgtggggggcatgactgagtataagttagtagtcgttggtgccgatggagtaggcaaatcggcgttaacgatccagcttattcagaatcatttcgtagatgagttgctgtccgttggcggagtcggggccgacggcgtgggcaaattgcttagtgtgggggggatgaccgagtataagttagtcgtcgtgggtgctgtgggcgtaggtaaatccgccctgactatacagcttattcaaaaccattttgtggatgagttgctgtctgtaggaggagtgggagctgtgggcgttgggaaattgctgagtgtgggagggatgactgagtacaagctggtggtcgtcggtgcccggggcgttggtaagagcgccctgaccatacaactcattcagaaccactttgtagacgaactgctaagcgtggggggcgtgggagcaaggggagtcgggaaactcctcagcgtggggggtacagaatacaagctggttgtagtcggcgccggttgtgtcgggaagtccgcactcacaattcagcttatccagaatcacttcgtggacgagtatctgttgtcagtgggcggtggggcaggctgcgttggaaagagcctgctctcagtgggcggcacagagtacaaattagtggtcgtgggagctggggacgtgggaaagtccgctctcactatccagctgattcagaaccattttgtcgacgaatatctcctgtcagtggggggcggggctggggacgtgggcaaatcacttctaagcgtgggcggaacctgcctgctggatatcctggacacagcgggacacgaagaatacagcgccatgagagatcagtacatgcgaacgggtgaaggcttcctgtgcctcctgtctgttggtggcacagcagggcacgaggaatactccctgctgtccgtgggtgggactgaatataagctggtagttgttggcgcaggaagagttgggaagagtgctctaaccatccaacttatccagaaccactttgtggacgagtatctgctttctgttggtggcggcgccgggcgcgtcggaaagtct

SEQ ID NO: 14 - RNA, 936 nt, synthetic construct
  source 1..936: mol_type = other RNA
atgacagaatacaagttagtggtggtaggagcgtgtggagtggggaaaagcgccctaactattcagttgatacaaaaccatttcgtggacgaagtgggcgcatgtggagtcggcaagatgaccgagtataagctcgtagtcgtgggggccgacggcgtggggaaatcagcccttactatccagcttatacaaaaccattttgttgacgaagtaggcgccgacggggtcggaaagatgaccgagtacaagctggttgtcgtcggggccgtaggggttggcaaaagcgccctgactatccagctcatccaaaatcatttcgtcgacgaggtgggcgccgttggagtcggtaaaatgacggaatacaagttggtggtagtgggggctcggggggtgggcaagagtgctttgaccatacagttaattcagaaccactttgtggatgaggtcggcgctcgcggagtcggcaaaaccgagtataaattggttgttgtcggcgcaggctgtgtgggcaagtctgctctcaccattcaactaatccagaaccactttgttgatgagtacggagcaggctgcgtgggaaaaagcacggagtacaagctggttgtcgtaggagcaggagacgtgggcaaatccgctctgacaattcagcttatccagaatcactttgtggatgaatatggtgccggggatgtcggtaaaagtacctgcttactggacatcctggacactgctggtcacgaggaatactccgcgatgcgagatcagtatatgcggacaggagagggtttcctgtgcacagcaggccatgaggaatattctactgaatataagctggtggtggtgggggctggtagagtggggaagtcagcactgacaatccagctcattcagaatcacttcgttgatgagtacggtgcgggtagagtgggcaagtcc

SEQ ID NO: 15 - RNA, 1206 nt, synthetic construct
  source 1..1206: mol_type = other RNA
atgacggaatacaagctcgtcgtggtgggcgccgatggcgtcggcaagtcggcactgactatccagctcatacaaaatcattttgttgatgaacttttatccgtcggtggagtgggggcggacggagtggggaagctgctgagcgttggtgggatgaccgaatacaaactggtggtggtcggggcgtgtggggtgggcaaatcagccctcacaatacagctgattcagaaccactttgttgacgagcttctgtccgtgggcggggtgggggcatgtggagtggggaagctcctgtccgtagggggcatgactgagtacaagctggttgttgtaggtgctgttggagtgggaaagagcgccctcacaatccaattgatccagaatcacttcgtcgacgagctcttgagcgttggaggtgtgggggccgtaggcgtgggcaagctgctatctgtgggtggaatgaccgaatataagttggtagtggtgggggctcgcggagtcgggaagagtgctcttactattcagctgattcagaaccatttcgtagatgaactcctatcagtcggtggcgtgggcgccagaggcgtgggcaaactgttatcagtgggcggtactgagtacaaactagtagtcgtaggagctgggtgtgtgggcaaaagtgcactcaccatccagctgatccagaaccatttcgtggatgagtatttgttgagtgtcggaggtggagctggctgcgtgggcaaaagcctgctgtccgttggggggacggaatacaaactagttgttgtgggggctggtgatgtcggaaagtccgccttgactattcagcttattcaaaatcactttgtcgacgagtatcttctgagcgtcggcggcggcgcaggtgacgtaggaaagtctttactgtcagttgggggaacctgccttctggacatcctggacacagccggccacgaggaatacagcgccatgcgggatcagtatatgaggacaggagaaggcttcctctgcctcttaagtgtcggcggtacagctggccacgaggagtattctctgctgtctgtcggaggcaccgagtacaaacttgtggtggttggagcagggcgtgtcggcaaatctgccctgaccatccagctgatacaaaaccattttgtggatgagtatttactttccgtgggaggcggtgcgggacgagtgggcaagagc

SEQ ID NO: 16 - RNA, 1224 nt, synthetic construct
  source 1..1224: mol_type = other RNA
tataagttggtggtggttggggcatgtggcgtaggtaagagcgcactgacttataagctagttgtcgtgggagctgacggagtggggaagagtgcactcacatacaagctcgtcgtggtgggcgccgtcggcgtagggaaatcagctcttacctataaactcgtggtggtgggcgctcgcggtgtgggcaaaagtgccctgaccaagctggtcgtcgtgggcgccggctgcgttgggaaatccgccctgacaatcaaattggtcgtcgtgggcgccggggacgtgggtaagtcagccttgaccatcctcgacatactggacactgccggccacgaagaatattctgccatgagggataaattagtggtggtaggtgccggtagagtggggaagtcggctctcaccatttacaagctggtggtagtcggcgcatgtggagtgggaaaaagcgccctgacatacaaattagtggtggtcggtgctgatggggtcggcaagtcagctctgacctacaaactcgttgtcgttggggccgtcggagtgggtaaatccgccctgacctataaattggtcgtggtaggggctcggggagtcgggaaatctgcgctgaccaagctcgttgtggtgggagcaggttgcgttggtaagtctgctttgactatcctcgacatcctcgacaccgcaggacatgaggagtactccgccatgcgtgacaagctagtagttgtaggagccggtgatgtgggaaagtccgctctgacaattaaattggtagtcgtgggggccggcagagtgggaaaaagcgcgctgacaatctataaacttgttgtggtgggcgcagtcggagttggtaagtcagctctgacttacaagctcgttgttgtgggcgctcgcggggtgggcaagtctgccctaacaaaactagtggtggtcggcgccgggtgcgtaggaaagagtgctctgaccatacttgacattctggatactgcgggccacgaggaatatagcgcgatgcgagattacaaactggttgttgtgggagcatgtggcgtcgggaagtccgcattaacgtacaagttagtggtggtcggagcggatggcgttgggaagtctgcacttacgaagttggtggtggtgggggccggagacgtaggcaagtccgcacttaccatcaaactggttgtcgtcggcgccgggcgggtgggcaagagcgcactgactatt

SEQ ID NO: 17 - RNA, 1053 nt, synthetic construct
  source 1..1053: mol_type = other RNA
atgacggaatacaagctcgtcgtggtgggcgccgatggcgtcggcaagtcggcactgactatccagctcatacaaaatcattttgttgatgaacttttatccgtcggtggagtgggggcggacggagtggggaagctgctgagcgttggtgggatgaccgaatacaaactggtggtggtcggggcgtgtggggtgggcaaatcagccctcacaatacagctgattcagaaccactttgttgacgagcttctgtccgtgggcggggtgggggcatgtggagtggggaagctcctgtccgtagggggcatgactgagtacaagctggttgttgtaggtgctgttggagtgggaaagagcgccctcacaatccaattgatccagaatcacttcgtcgacgagctcttgagcgttggaggtgtgggggccgtaggcgtgggcaagctgctatctgtgggtggaatgaccgaatataagttggtagtggtgggggctcgcggagtcgggaagagtgctcttactattcagctgattcagaaccatttcgtagatgaactcctatcagtcggtggcgtgggcgccagaggcgtgggcaaactgttatcagtgggcggtactgagtacaaactagtagtcgtaggagctgggtgtgtgggcaaaagtgcactcaccatccagctgatccagaaccatttcgtggatgagtatttgttgagtgtcggaggtggagctggctgcgtgggcaaaagcctgctgtccgttggggggacggaatacaaactagttgttgtgggggctggtgatgtcggaaagtccgccttgactattcagcttattcaaaatcactttgtcgacgagtatcttctgagcgtcggcggcggcgcaggtgacgtaggaaagtctttactgtcagttgggggaacctgccttctggacatcctggacacagccggccacgaggaatacagcgccatgcgggatcagtatatgaggacaggagaaggcttcctctgcctcttaagtgtcggcggtacagctggccacgaggagtattct

SEQ ID NO: 18 - RNA, 1665 nt, synthetic construct
  source 1..1665: mol_type = other RNA
atgacagagtataaactcgtggtggtcggcgcagatggcgttggaaagagcgccctgactattcagctcatccagaaccatttcgtagacgaactcctgtctgtaggaggagtcggcgcagacggtgtgggaaagctgctgtctgtgggcggtatgaccgaatacaagttagtggtggtcggggcctgcggtgtgggtaaaagcgctctgaccatacagcttatccagaaccacttcgtggatgagctcctgtctgttgggggagtgggggcttgcggagttggcaagctgctttcagtgggcgggatgacagaatataaattggtggtagtgggggccgtaggcgtcggcaagagcgccctgactatacaactgatccaaaaccacttcgtggacgagttgctctccgtgggtggcgtgggcgccgttggagtcgggaaactcctgtccgtcggtggcatgactgagtataaacttgttgtagtcggggcacggggcgtgggaaagtccgctttaaccatacaattgatacagaaccactttgttgatgaactgctgtctgtgggtggggtgggtgccaggggagtcggtaagctgttgtcagtaggagggacctgtctcctagacattctggacacagccggccacgaggagtactcagctatgcgcgatcagtatatgagaacaggggagggatttctgtgtctcctctccgtcggagggacagcagggcacgaagagtacagcctgctttcggtggggggcactgagtacaagctagtcgtggttggcgcaggcgacgtgggtaaatctgccctcaccattcagttaattcagaatcatttcgtcgatgaatatctgctgagtgttggcggcggcgctggagacgtcggcaaatcccttctgagcgtcgggggaatgacggagtataaactggtagtcgtaggtgctgccggcgtcggcaaaagcgcgctaaccatccagctgatccagaatcactttgtggatgaattgttgagtgttgggggggtgggagccgctggagtggggaagttactaagtgtcggtggcagatcttacggaatccctttcatcgaaacctcaaccaagactcgtcaaagggttgaagacgctttctatacacttgtcagagagattaggcagttgctttccgtgggtggagaaacctccacaaagacgcggcaactcctcagtgtcgggggcactgagtacaagctggtggtagtgggagccggttgcgtgggcaagtcagcgctgacaatccagctcatccagaatcattttgttgatgagtacttgttatcagttggcggaggcgcaggctgcgtggggaaatcgcttctgtccgttggaggtatgactgagtacaagctggtggtggtgggggcgtctggggtggggaaatctgcactgacgattcaactgatccagaatcattttgtagacgagttactgagtgtgggaggcgtgggcgcgtccggcgtgggtaagttgctaagcgtgggggggacctgtttgctggatattctggacactgccggacgagaagaatacagtgcaatgagggatcagtatatgcgaaccggagagggctttctttgtctgctcagcgtcggcggaacagccggccgcgaagagtacagc

SEQ ID NO: 19 - RNA, 1206 nt, synthetic construct
  source 1..1206: mol_type = other RNA
atgacggaatataagctcgtggtcgttggggcctgtggcgtgggtaagagcgcactaaccattcagttaatccagaaccactttgttgatgaacttctcagcgtgggcggtgttggggcctgcggcgttggaaaactgttgtccgtgggtggcatgacagagtacaaactggtcgtggtgggggctgtgggtgtgggcaagtccgccctgactatccaactgatacagaaccatttcgtagatgaactgttgagtgtgggcggagtcggcgcagtcggggtaggtaaattgctgagcgtgggaggcatgactgagtataaactcgtggttgtgggtgcagatggagtaggaaagagtgccctcacaatacaactgattcagaatcattttgtcgatgaactgctgagcgtcggcggagtgggagctgatggtgttggcaagctgctttcagtagggggaacctgcctgctggacatcctggacaccgcggggcacgaagagtatagcgctatgcgggaccagtacatgagaaccggtgagggctttctgtgtctgctgtctgtcggaggaacagccggccacgaggagtattctctactatcagtaggcgggactgagtacaagctggtggtggttggcgctggctgtgtggggaaatctgcattaacgattcaactgatccagaatcacttcgtcgatgagtacctcctctcagtcggcggaggcgcaggctgcgtgggcaagtcactcttaagcgtgggcggcacagagtacaagctggttgtcgttggggcaggggacgtcgggaagtccgccctgactatccagctgattcagaaccattttgtggacgaatatctgctgtccgtcggggggggtgctggagacgtgggaaaatccttgctcagtgtaggaggaatgacagagtacaaacttgtggtggttggcgctgctggagtaggaaaatccgccctgaccatacaacttattcagaatcacttcgtggacgagttgctcagtgtagggggtgtcggggcggccggggtggggaagttgctttcggttgggggtatgactgaatacaagttggtggtggtgggcgcgagcggggtgggtaagtctgccttgaccatccagctgatccagaaccatttcgttgatgaacttctttctgtgggaggagtcggcgcctcgggcgtggggaaa

SEQ ID NO: 20 - RNA, 1206 nt, synthetic construct
  source 1..1206: mol_type = other RNA
atgacagagtacaaactggtagtagtgggagcagatggggttggcaaatccgcgctcaccatccagctgattcaaaaccatttcgtcgatgagctgctgagcgttgggggagtgggcgctgacggggtgggcaagctcctttctgtaggcggaatgaccgagtataaactcgtagtggtcggagctgtgggagtgggaaagtcagcgctcactatccaactaatacagaatcactttgtcgacgaactgctttctgtcggtggagtgggcgccgtgggtgtcggcaagctgcttagcgtggggggaatgacagaatacaagttggtcgttgttggtgcccgcggagtgggcaaatctgcactgaccatccaactgatccagaaccactttgtggatgagctactttccgtgggaggcgtcggtgccaggggtgtgggaaagctcctgagtgttggtggcacatgtttgttagacattctcgacacggctgggcatgaggagtatagcgctatgcgggatcagtatatgagaaccggcgaaggctttctctgccttctctcagtgggcggcacagccgggcacgaggaatattccctgctttcggtgggggggacttgcttgctggacatattagacacagcagggagggaagagtattccgcaatgcgagatcagtacatgcgtactggggaagggttcctgtgcctgctgagtgtgggaggcaccgccgggagggaggaatacagccttctgtcagtcggcggcactgagtacaagctggttgtggtgggagccggatgtgtcggtaaaagtgccctaaccatccagctcattcagaaccacttcgtcgatgagtatttgctgtccgtaggcggcggggccggctgtgttgggaaaagcctgctgagcgtaggagggatgactgagtacaagctcgttgtcgtgggtgcatgcggagtagggaagtcagcgctgacaattcagttaatccagaatcacttcgtcgatgaacttctaagtgtgggtggagtcggcgcctgtggagtgggtaagttgctctccgttggaggcacggagtacaagctggtcgtggttggcgccggcgacgtgggcaaaagcgctttgaccattcaactgatccagaatcattttgtggatgaatacttgctctctgtgggtggcggggcaggggacgtggggaaatct

SEQ ID NO: 21 - RNA, 1206 nt, synthetic construct
  source 1..1206: mol_type = other RNA
atgactgaatataaattagttgtggtgggagccgatggagtggggaaatccgcgttgacaatccagctgatccagaaccattttgttgacgaactcctctcagtcgggggcgttggagctgatggcgttggcaagctgctgtctgtggggggaatgacggaatacaagctggtcgtcgtgggcgcggtcggggtaggtaagtccgcgctgacaattcagctgattcagaatcacttcgtggatgagctgctgagcgtaggcggcgtgggcgccgtgggagtcggtaagttgctgtctgtgggtggtaccgagtacaagctcgtggttgtcggtgctggtgacgtgggcaagtcggctctgacgatccaactgatccagaatcatttcgttgatgagtaccttctgagcgtagggggcggggccggagatgtcggcaaatctctcctcagcgttggcgggaggtcatacgggatacctttcattgagacttccaccaaaactcgtcaacgcgtcgaggatgccttttataccctagtgagagagatccgacagctgctgagtgtgggcggtgaaacaagcaccaagacccggcagttactttctgtggggggcatgacagaatacaagttggtagttgtcggcgcctgcggggtggggaaaagtgctctaaccattcaattaatccaaaatcactttgtcgacgagctccttagtgtcggaggcgtgggcgcctgcggggtaggcaaactcctttcagtcggcggaatgaccgaatataaactggtggtggtgggagctgcaggagttggaaaaagcgctctgactatccaactgattcagaaccatttcgtggacgagctgctcagtgtggggggtgtcggggcagccggcgtgggaaaacttctgagcgtgggcgggatgaccgaatacaagctggtggtggttggcgcatccggtgtgggaaagtcagccctgacaatccagcttatacagaaccacttcgtcgacgaacttttgtctgttgggggtgtgggcgcatctggagtcggaaagctactatcggttggaggtacagagtataaattggtggtggtaggtgctggatgtgtcggcaagtccgcacttactatacagctcattcagaaccactttgtagacgagtatttactcagcgtgggcgggggggcagggtgcgtaggcaagtcc

SEQ ID NO: 22 - RNA, 1053 nt, synthetic construct
  source 1..1053: mol_type = other RNA
atgacggaatataagttggtcatggtgggtgcggatggtgtaggcaaatcagctctgactatccaactgatccagaatcactttgtggacgaactgttatcagttgggggggtcggggctgacggtgtcggaaaactgctaagtgtcggcggcatgaccgaatataagctcgttgtggtgggggcttgtggagttggcaagtccgctctgactatccaactaattcagaatcatttcgtcgatgagctgctgagcgttggaggagtcggcgcttgcggagtaggcaaacttctgtcagtgggaggcatgaccgaatacaagctcgtggtggtaggcgccgttggtgtagggaagagtgcgctaaccattcaacttatccagaaccattttgttgacgagcttctctccgtcggtggtgtaggcgcggtgggagtgggcaagctgctctccgtcggcggcatgaccgagtataaacttgtagtggtgggcgccaggggcgtggggaaaagcgccctgacaatccagctcatacagaatcacttcgtagacgagctgctgagtgttggaggagtgggcgcccgcggcgttggtaagctgctcagcgtgggggggactgaatataaactcgtcgtcgtgggagcaggctgtgtggggaagagtgccttgaccattcagttgatccagaaccacttcgtcgacgagtatctgttgtctgtgggaggcggagcagggtgcgttggaaagagccttctctctgtgggtggcacagaatacaaactggttgtcgtgggggccggcgacgtgggcaagagcgccttgactattcaactcatacagaaccactttgtggatgagtaccttctctctgtcgggggaggcgccggggacgtggggaaaagcctcctgtcggtgggaggaacgtgtcttctggatattttagacaccgcagggcatgaggagtactccgcaatgcgagatcagtacatgaggacaggtgagggtttcctgtgcttgctgtccgtgggaggtacagccggccacgaggaatactct

SEQ ID NO: 23 - protein, 137 aa, synthetic construct
MTEYKLVVVGARGVGKSALTIQLEYKLVVVGADGVGKSALTIEYKLVVVGAVGVGKSALTIEYKLVVVGACGVGKSALTIYKLVVVGAGDVGKSALTIQYKLVVVGAGCVGKSALTIQLLDILDTAGHEEYSAMRDQ

SEQ ID NO: 24 - protein, 119 aa, synthetic construct
YKLVVVGACGVGKSALTYKLVVVGADGVGKSALTYKLVVVGAVGVGKSALTYKLVVVGARGVGKSALTKLVVVGAGCVGKSALTIKLVVVGAGDVGKSALTILDILDTAGHEEYSAMRD

SEQ ID NO: 25 - protein, 167 aa, synthetic construct
LLSVGGYKLVVVGACGVGKSALTLLSVGGYKLVVVGADGVGKSALTLLSVGGYKLVVVGAVGVGKSALTLLSVGGYKLVVVGARGVGKSALTLLSVGGKLVVVGAGCVGKSALTILLSVGGKLVVVGAGDVGKSALTILLSVGGLDILDTAGHEEYSAMRDLLSVGG

SEQ ID NO: 26 - protein, 84 aa, synthetic construct
VGACGVGKSLLSVGGVVVGACGVGLLSVGGVGACGVGKSLLSVGGVVVGACGVGLLSVGGVGACGVGKSLLSVGGVVVGACGVG

SEQ ID NO: 27 - protein, 84 aa, synthetic construct
VGACGVGKSLLSVGGVVVGACGVGLLSVGGVGADGVGKSLLSVGGVVVGADGVGLLSVGGGAGDVGKSALLSVGGVVGAGDVGK

SEQ ID NO: 28 - protein, 147 aa, synthetic construct
MTEYKLVVVGACGVGKSALTIQLIQNHFVDELLSVGGVGACGVGKLLSVGGMTEYKLVVVGADGVGKSALTIQLIQNHFVDELLSVGGVGADGVGKLLSVGGTEYKLVVVGAGDVGKSALTIQLIQNHFVDEYLLSVGGGAGDVGKS

SEQ ID NO: 29 - protein, 216 aa, synthetic construct
MTEYKLVVVGAGCVGKSALTIQLIQNHFVDELLSVGGMTEYKLVVVGACGVGKSALTIQLIQNHFVDELLSVGGMTEYKLVVVGADGVGKSALTIQLIQNHFVDELLSVGGMTEYKLVVVGAGDVGKSALTIQLIQNHFVDELLSVGGMTEYKLVVVGAVGVGKSALTIQLIQNHFVDELLSVGGTCLLDILDTAGHEEYSAMRDQYMRTGEGFLC

SEQ ID NO: 30 - protein, 117 aa, synthetic construct
MTEYKLVVVGACGVGKSALTIQLIQNHFVDEVGACGVGKMTEYKLVVVGADGVGKSALTIQLIQNHFVDEVGADGVGKTEYKLVVVGAGDVGKSALTIQLIQNHFVDEYGAGDVGKS

SEQ ID NO: 31 - protein, 393 aa, synthetic construct
CINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRMTEYKLVVVGAGCVGKSALTIQLIQNHFVDELLSVGGMTEYKLVVVGACGVGKSALTIQLIQNHFVDELLSVGGMTEYKLVVVGADGVGKSALTIQLIQNHFVDELLSVGGMTEYKLVVVGAGDVGKSALTIQLIQNHFVDELLSVGGMTEYKLVVVGAVGVGKSALTIQLIQNHFVDELLSVGGTCLLDILDTAGHEEYSAMRDQYMRTGEGFLC

SEQ ID NO: 32 - protein, 399 aa, synthetic construct
MTEYKLVVVGAGCVGKSALTIQLIQNHFVDELLSVGGMTEYKLVVVGACGVGKSALTIQLIQNHFVDELLSVGGMTEYKLVVVGADGVGKSALTIQLIQNHFVDELLSVGGMTEYKLVVVGAGDVGKSALTIQLIQNHFVDELLSVGGMTEYKLVVVGAVGVGKSALTIQLIQNHFVDELLSVGGTCLLDILDTAGHEEYSAMRDQYMRTGEGFLCLLSVGGCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNR

SEQ ID NO: 33 - protein, 399 aa, synthetic construct; identical to SEQ ID NO: 32

SEQ ID NO: 34 - protein, 136 aa, synthetic construct
YKLVVVGACGVGKSALTYKLVVVGADGVGKSALTYKLVVVGAVGVGKSALTYKLVVVGARGVGKSALTKLVVVGAGCVGKSALTIKLVVVGAGDVGKSALTILDILDTAGHEEYSAMRDKLVVVGAGRVGKSALTI

SEQ ID NO: 35 - protein, 402 aa, synthetic construct
MTEYKLVVVGACGVGKSALTIQLIQNHFVDELLSVGGVGACGVGKLLSVGGMTEYKLVVVGADGVGKSALTIQLIQNHFVDELLSVGGVGADGVGKLLSVGGMTEYKLVVVGAVGVGKSALTIQLIQNHFVDELLSVGGVGAVGVGKLLSVGGMTEYKLVVVGARGVGKSALTIQLIQNHFVDELLSVGGVGARGVGKLLSVGGTEYKLVVVGAGCVGKSALTIQLIQNHFVDEYLLSVGGGAGCVGKSLLSVGGTEYKLVVVGAGDVGKSALTIQLIQNHFVDEYLLSVGGGAGDVGKSLLSVGGTCLLDILDTAGHEEYSAMRDQYMRTGEGFLCLLSVGGTAGHEEYSLLSVGGTEYKLVVVGAGRVGKSALTIQLIQNHFVDEYLLSVGGGAGRVGKS

SEQ ID NO: 36 - protein, 312 aa, synthetic construct
MTEYKLVVVGACGVGKSALTIQLIQNHFVDEVGACGVGKMTEYKLVVVGADGVGKSALTIQLIQNHFVDEVGADGVGKMTEYKLVVVGAVGVGKSALTIQLIQNHFVDEVGAVGVGKMTEYKLVVVGARGVGKSALTIQLIQNHFVDEVGARGVGKTEYKLVVVGAGCVGKSALTIQLIQNHFVDEYGAGCVGKSTEYKLVVVGAGDVGKSALTIQLIQNHFVDEYGAGDVGKSTCLLDILDTAGHEEYSAMRDQYMRTGEGFLCTAGHEEYSTEYKLVVVGAGRVGKSALTIQLIQNHFVDEYGAGRVGKS

SEQ ID NO: 37 - protein, 402 aa, synthetic construct
MTEYKLVVVGADGVGKSALTIQLIQNHFVDELLSVGGVGADGVGKLLSVGGMTEYKLVVVGACGVGKSALTIQLIQNHFVDELLSVGGVGACGVGKLLSVGGMTEYKLVVVGAVGVGKSALTIQLIQNHFVDELLSVGGVGAVGVGKLLSVGGMTEYKLVVVGARGVGKSALTIQLIQNHFVDELLSVGGVGARGVGKLLSVGGTEYKLVVVGAGCVGKSALTIQLIQNHFVDEYLLSVGGGAGCVGKSLLSVGGTEYKLVVVGAGDVGKSALTIQLIQNHFVDEYLLSVGGGAGDVGKSLLSVGGTCLLDILDTAGHEEYSAMRDQYMRTGEGFLCLLSVGGTAGHEEYSLLSVGGTEYKLVVVGAGRVGKSALTIQLIQNHFVDEYLLSVGGGAGRVGKS

SEQ ID NO: 38 - protein, 408 aa, synthetic construct
YKLVVVGACGVGKSALTYKLVVVGADGVGKSALTYKLVVVGAVGVGKSALTYKLVVVGARGVGKSALTKLVVVGAGCVGKSALTIKLVVVGAGDVGKSALTILDILDTAGHEEYSAMRDKLVVVGAGRVGKSALTIYKLVVVGACGVGKSALTYKLVVVGADGVGKSALTYKLVVVGAVGVGKSALTYKLVVVGARGVGKSALTKLVVVGAGCVGKSALTILDILDTAGHEEYSAMRDKLVVVGAGDVGKSALTIKLVVVGAGRVGKSALTIYKLVVVGAVGVGKSALTYKLVVVGARGVGKSALTKLVVVGAGCVGKSALTILDILDTAGHEEYSAMRDYKLVVVGACGVGKSALTYKLVVVGADGVGKSALTKLVVVGAGDVGKSALTIKLVVVGAGRVGKSALTI

SEQ ID NO: 39 - protein, 351 aa, synthetic construct
MTEYKLVVVGADGVGKSALTIQLIQNHFVDELLSVGGVGADGVGKLLSVGGMTEYKLVVVGACGVGKSALTIQLIQNHFVDELLSVGGVGACGVGKLLSVGGMTEYKLVVVGAVGVGKSALTIQLIQNHFVDELLSVGGVGAVGVGKLLSVGGMTEYKLVVVGARGVGKSALTIQLIQNHFVDELLSVGGVGARGVGKLLSVGGTEYKLVVVGAGCVGKSALTIQLIQNHFVDEYLLSVGGGAGCVGKSLLSVGGTEYKLVVVGAGDVGKSALTIQLIQNHFVDEYLLSVGGGAGDVGKSLLSVGGTCLLDILDTAGHEEYSAMRDQYMRTGEGFLCLLSVGGTAGHEEYS

SEQ ID NO: 40 - protein, 555 aa, synthetic construct
MTEYKLVVVGADGVGKSALTIQLIQNHFVDELLSVGGVGADGVGKLLSVGGMTEYKLVVVGACGVGKSALTIQLIQNHFVDELLSVGGVGACGVGKLLSVGGMTEYKLVVVGAVGVGKSALTIQLIQNHFVDELLSVGGVGAVGVGKLLSVGGMTEYKLVVVGARGVGKSALTIQLIQNHFVDELLSVGGVGARGVGKLLSVGGTCLLDILDTAGHEEYSAMRDQYMRTGEGFLCLLSVGGTAGHEEYSLLSVGGTEYKLVVVGAGDVGKSALTIQLIQNHFVDEYLLSVGGGAGDVGKSLLSVGGMTEYKLVVVGAAGVGKSALTIQLIQNHFVDELLSVGGVGAAGVGKLLSVGGRSYGIPFIETSTKTRQRVEDAFYTLVREIRQLLSVGGETSTKTRQLLSVGGTEYKLVVVGAGCVGKSALTIQLIQNHFVDEYLLSVGGGAGCVGKSLLSVGGMTEYKLVVVGASGVGKSALTIQLIQNHFVDELLSVGGVGASGVGKLLSVGGTCLLDILDTAGREEYSAMRDQYMRTGEGFLCLLSVGGTAGREEYS

SEQ ID NO: 41 - protein, 402 aa, synthetic construct
MTEYKLVVVGACGVGKSALTIQLIQNHFVDELLSVGGVGACGVGKLLSVGGMTEYKLVVVGAVGVGKSALTIQLIQNHFVDELLSVGGVGAVGVGKLLSVGGMTEYKLVVVGADGVGKSALTIQLIQNHFVDELLSVGGVGADGVGKLLSVGGTCLLDILDTAGHEEYSAMRDQYMRTGEGFLCLLSVGGTAGHEEYSLLSVGGTEYKLVVVGAGCVGKSALTIQLIQNHFVDEYLLSVGGGAGCVGKSLLSVGGTEYKLVVVGAGDVGKSALTIQLIQNHFVDEYLLSVGGGAGDVGKSLLSVGGMTEYKLVVVGAAGVGKSALTIQLIQNHFVDELLSVGGVGAAGVGKLLSVGGMTEYKLVVVGASGVGKSALTIQLIQNHFVDELLSVGGVGASGVGK

SEQ ID NO: 42 - protein, 402 aa, synthetic construct
MTEYKLVVVGADGVGKSALTIQLIQNHFVDELLSVGGVGADGVGKLLSVGGMTEYKLVVVGAVGVGKSALTIQLIQNHFVDELLSVGGVGAVGVGKLLSVGGMTEYKLVVVGARGVGKSALTIQLIQNHFVDELLSVGGVGARGVGKLLSVGGTCLLDILDTAGHEEYSAMRDQYMRTGEGFLCLLSVGGTAGHEEYSLLSVGGTCLLDILDTAGREEYSAMRDQYMRTGEGFLCLLSVGGTAGREEYSLLSVGGTEYKLVVVGAGCVGKSALTIQLIQNHFVDEYLLSVGGGAGCVGKSLLSVGGMTEYKLVVVGACGVGKSALTIQLIQNHFVDELLSVGGVGACGVGKLLSVGGTEYKLVVVGAGDVGKSALTIQLIQNHFVDEYLLSVGGGAGDVGKS

SEQ ID NO: 43 - protein, 402 aa, synthetic construct
MTEYKLVVVGADGVGKSALTIQLIQNHFVDELLSVGGVGADGVGKLLSVGGMTEYKLVVVGAVGVGKSALTIQLIQNHFVDELLSVGGVGAVGVGKLLSVGGTEYKLVVVGAGDVGKSALTIQLIQNHFVDEYLLSVGGGAGDVGKSLLSVGGRSYGIPFIETSTKTRQRVEDAFYTLVREIRQLLSVGGETSTKTRQLLSVGGMTEYKLVVVGACGVGKSALTIQLIQNHFVDELLSVGGVGACGVGKLLSVGGMTEYKLVVVGAAGVGKSALTIQLIQNHFVDELLSVGGVGAAGVGKLLSVGGMTEYKLVVVGASGVGKSALTIQLIQNHFVDELLSVGGVGASGVGKLLSVGGTEYKLVVVGAGCVGKSALTIQLIQNHFVDEYLLSVGGGAGCVGKS

SEQ ID NO: 44 - RNA, 69 nt, synthetic construct
  source 1..69: mol_type = other RNA
atgacagagtacaagctggtggtcgtgggggccaggggcgtgggcaagagcgccctgactatccagctg

SEQ ID NO: 45 - protein, 351 aa, synthetic construct
MTEYKLVMVGADGVGKSALTIQLIQNHFVDELLSVGGVGADGVGKLLSVGGMTEYKLVVVGACGVGKSALTIQLIQNHFVDELLSVGGVGACGVGKLLSVGGMTEYKLVVVGAVGVGKSALTIQLIQNHFVDELLSVGGVGAVGVGKLLSVGGMTEYKLVVVGARGVGKSALTIQLIQNHFVDELLSVGGVGARGVGKLLSVGGTEYKLVVVGAGCVGKSALTIQLIQNHFVDEYLLSVGGGAGCVGKSLLSVGGTEYKLVVVGAGDVGKSALTIQLIQNHFVDEYLLSVGGGAGDVGKSLLSVGGTCLLDILDTAGHEEYSAMRDQYMRTGEGFLCLLSVGGTAGHEEYS

SEQ ID NO: 46 - RNA, 57 nt, synthetic construct
  source 1..57: mol_type = other RNA
gaatacaagctggtggtggtgggcgccgacggcgtggggaagtccgccctcacaatc

SEQ ID NO: 47 - RNA, 57 nt, synthetic construct
  source 1..57: mol_type = other RNA
gagtacaagctggtcgtggtgggggccgtgggcgtcgggaagtccgccctgacaatc

SEQ ID NO: 48 - RNA, 57 nt, synthetic construct
  source 1..57: mol_type = other RNA
gagtacaagttggtggtggtgggcgcctgcggggtgggcaagagcgccctgaccatc

SEQ ID NO: 49 - RNA, 57 nt, synthetic construct
  source 1..57: mol_type = other RNA
tacaagctcgtggtggtgggggccggggacgtggggaagagtgccctgaccatccag

SEQ ID NO: 50 - RNA, 57 nt, synthetic construct
  source 1..57: mol_type = other RNA
tataagctggtggtggtcggcgccgggtgcgtgggcaagtccgccctgacaatccag

SEQ ID NO: 51 - RNA, 57 nt, synthetic construct
  source 1..57: mol_type = other RNA
ctgctggatatcctggataccgccggccacgaggagtacagcgccatgagggatcag

SEQ ID NO: 52 - RNA, 51 nt, synthetic construct
  source 1..51: mol_type = other RNA
tacaagctggtcgtcgtgggggcctgcggcgtggggaagtccgctctgacc

SEQ ID NO: 53 - RNA, 51 nt, synthetic construct
  source 1..51: mol_type = other RNA
tacaaactggtggtggtgggggccgatggggtgggcaagtccgccctgacc

SEQ ID NO: 54 - RNA, 51 nt, synthetic construct
  source 1..51: mol_type = other RNA
tacaagctggtggtggtcggggccgtcggcgtgggcaaaagcgccctgaca

SEQ ID NO: 55 - RNA, 51 nt, synthetic construct
  source 1..51: mol_type = other RNA
tacaagctcgtggtggtgggcgccaggggggtgggcaagtccgccctgaca

SEQ ID NO: 56 - RNA, 51 nt, synthetic construct
  source 1..51: mol_type = other RNA
aagctggtcgtggtgggcgccgggtgcgtgggcaagagcgccctgacaatc

SEQ ID NO: 57 - RNA, 51 nt, synthetic construct
  source 1..51: mol_type = other RNA
aagctggtggtggtgggggccggggatgtgggcaagagcgccctgaccatc

SEQ ID NO: 58 - RNA, 51 nt, synthetic construct
  source 1..51: mol_type = other RNA
ctggacatcctggacacagccgggcacgaggagtacagcgccatgagggat

SEQ ID NO: 59 - RNA, 51 nt, synthetic construct
  source 1..51: mol_type = other RNA
tacaagctcgtggtggtgggcgcctgcggcgtggggaagtccgccctgaca

SEQ ID NO: 60 - RNA, 51 nt, synthetic construct
  source 1..51: mol_type = other RNA
tacaagctggtggtcgtgggcgccgacggggtgggcaagagcgccctcacc

SEQ ID NO: 61 - RNA, 51 nt, synthetic construct
  source 1..51: mol_type = other RNA
tacaagctggtcgtggtcggggccgtgggcgtgggcaagtccgctctgaca

SEQ ID NO: 62 - RNA, 51 nt, synthetic construct
  source 1..51: mol_type = other RNA
tacaagctggtggtggtgggggccaggggggtcgggaaatccgccctgact

SEQ ID NO: 63 - RNA, 51 nt, synthetic construct
  source 1..51: mol_type = other RNA
aagctcgtggtggtgggggccgggtgcgtggggaagtccgccctgacaatt

SEQ ID NO: 64 - RNA, 51 nt, synthetic construct
  source 1..51: mol_type = other RNA
aagctggtcgtggtgggcgccggggatgtgggcaagagcgccctgaccatc

SEQ ID NO: 65 - RNA, 51 nt, synthetic construct
  source 1..51: mol_type = other RNA
ctggacatcctggacaccgccggccacgaggagtacagcgccatgagggat

SEQ ID NO: 66 - RNA, 27 nt, synthetic construct
  source 1..27: mol_type = other RNA
gttggagcctgtggagttggtaaaagt

SEQ ID NO: 67 - RNA, 27 nt, synthetic construct
  source 1..27: mol_type = other RNA
gtagttgtgggagcttgtggagtaggt

SEQ ID NO: 68 - RNA, 27 nt, synthetic construct
  source 1..27: mol_type = other RNA
gtaggagcttgtggagtaggaaagtct

SEQ ID NO: 69 - RNA, 27 nt, synthetic construct
  source 1..27: mol_type = other RNA
gtagtagtaggagcatgcggagtcgga

SEQ ID NO: 70 - RNA, 27 nt, synthetic construct
  source 1..27: mol_type = other RNA
gtaggagcatgcggagtaggaaagtca

SEQ ID NO: 71 - RNA, 27 nt, synthetic construct
  source 1..27: mol_type = other RNA
gtagtagtaggagcttgtggagtagga

SEQ ID NO: 72 - RNA, 27 nt, synthetic construct
  source 1..27: mol_type = other RNA
gtgggcgcctgtggagtaggaaaatct

SEQ ID NO: 73 - RNA, 27 nt, synthetic construct
  source 1..27: mol_type = other RNA
gtggtggtcggagcatgcggagtagga

SEQ ID NO: 74 - RNA, 27 nt, synthetic construct
  source 1..27: mol_type = other RNA
gtgggagctgacggcgtgggtaaatcc

SEQ ID NO: 75 - RNA, 27 nt, synthetic construct
  source 1..27: mol_type = other RNA
gtggtcgtgggagctgacggtgttggt

SEQ ID NO: 76 - RNA, 27 nt, synthetic construct
  source 1..27: mol_type = other RNA
ggagcgggagatgttggaaagtcagcc

SEQ ID NO: 77 - RNA, 27 nt, synthetic construct
  source 1..27: mol_type = other RNA
gttgtgggggctggtgacgtgggaaag

SEQ ID NO: 78 - RNA, 93 nt, synthetic construct
  source 1..93: mol_type = other RNA
atgacagaatacaaacttgtggtagttggagcatgcggtgtgggtaagagtgccctcaccatccaactaattcagaaccacttcgttgacgaa

SEQ ID NO: 79 - RNA, 24 nt, synthetic construct
  source 1..24: mol_type = other RNA
gttggagcatgtggtgtagggaag

SEQ ID NO: 80 - RNA, 93 nt, synthetic construct
  source 1..93: mol_type = other RNA
atgactgaatacaaacttgtggttgttggagcagatggggtcggaaaatccgccttgacaatacagcttattcagaatcactttgtggatgag

SEQ ID NO: 81 - RNA, 24 nt, synthetic construct
  source 1..24: mol_type = other RNA
gtcggagctgatggcgtcggaaag

SEQ ID NO: 82 - RNA, 93 nt, synthetic construct
  source 1..93: mol_type = other RNA
actgagtataaattagtggtagtcggggctggagacgtgggcaagagtgcccttaccatccagctcatccagaatcattttgtggacgagtat

SEQ ID NO: 83 - RNA, 24 nt, synthetic construct
  source 1..24: mol_type = other RNA
ggagctggtgacgtcggaaagtct

SEQ ID NO: 84 - RNA, 93 nt, synthetic construct
  source 1..93: mol_type = other RNA
atgactgaatataaactcgtcgtggttggagcaggttgcgttggcaaaagcgcccttacaatccagctgatacagaaccacttcgtggatgag

SEQ ID NO: 85 - RNA, 93 nt, synthetic construct
  source 1..93: mol_type = other RNA
atgacagagtacaagttggtggtggtgggcgcctgtggggtggggaaatctgcccttactatccagctgatccagaaccactttgtggatgag

SEQ ID NO: 86 - RNA, 93 nt, synthetic construct
  source 1..93: mol_type = other RNA
atgacagaatacaaactggtcgtggtcggcgctgatggcgttgggaagagtgctctgacaattcaactcatccagaatcacttcgtagacgaa

SEQ ID NO: 87 - RNA, 93 nt, synthetic construct
  source 1..93: mol_type = other RNA
atgacggagtataagctcgttgtggtaggcgccggagacgtaggcaagtccgcactgaccattcagctcattcaaaatcattttgtagacgaa

SEQ ID NO: 88 - RNA, 93 nt, synthetic construct
  source 1..93: mol_type = other RNA
atgaccgagtacaagctggttgttgtgggtgccgtgggggtgggtaagtcagccctcaccattcagctcatccaaaaccatttcgtggacgag

SEQ ID NO: 89 - RNA, 93 nt, synthetic construct
  source 1..93: mol_type = other RNA
acctgcctcctggacatattggacactgcggggcatgaggagtattccgcaatgcgcgatcagtacatgaggaccggagaaggatttctgtgc

SEQ ID NO: 90 - RNA, 93 nt, synthetic construct
  source 1..93: mol_type = other RNA
atgacagagtataagctggtcgtggtcggcgcgtgcggagtggggaaatcagctctcaccattcagctaatccagaaccacttcgttgacgaa

SEQ ID NO: 91 - RNA, 24 nt, synthetic construct
  source 1..24: mol_type = other RNA
gtcggcgcttgcggtgtcggaaag

SEQ ID NO: 92 - RNA, 93 nt, synthetic construct
  source 1..93: mol_type = other RNA
atgacggagtataaacttgtcgtagttggcgccgacggtgttggaaaatccgcattaaccatacaattgatccagaaccacttcgtggacgaa

SEQ ID NO: 93 - RNA, 24 nt, synthetic construct
  source 1..24: mol_type = other RNA
gtgggggctgacggcgtaggaaaa

SEQ ID NO: 94 - RNA, 93 nt, synthetic construct
  source 1..93: mol_type = other RNA
acagagtacaagctggtggtggttggcgcaggggatgtgggtaagagcgccctgactattcagctgatccaaaatcattttgtggatgaatac

SEQ ID NO: 95 - RNA, 24 nt, synthetic construct
  source 1..24: mol_type = other RNA
ggagcaggcgatgtggggaagtct

SEQ ID NO: 96 - RNA, 531 nt, synthetic construct
  source 1..531: mol_type = other RNA
tgtataaacctcgattgggatgtaatccgcgacaaaaccaagacaaaaattgaatctctcaaggagcacggccctatcaaaaataaaatgtcagagtcacccaataagacggtgtctgaggaaaaggccaagcaatacttggaagaatttcatcaaaccgccttggaacatccggaactaagcgagcttaagacagttacaggtactaacccagtgtttgccggagctaactacgccgcgtgggcggtcaatgtagcccaggtcattgatagtgaaactgctgacaatttagaaaagacaactgccgctctctccattctgcccggaatagggagcgttatgggtatcgcagatggcgcggtgcatcataacaccgaggagatcgtcgcacagtcgatcgctctcagtagcctaatggtggctcaggcaattcctctggtcggggaactggttgacatcggattcgccgcatacaatttcgtagagtctattataaatcttttccaggtggtccacaactcatataatcga

SEQ ID NO: 97 - RNA, 93 nt, synthetic construct
  source 1..93: mol_type = other RNA
atgactgagtacaagctggtagtggtaggggctggatgcgtcgggaagtcagccctgactatacagctgatccagaaccacttcgtggatgag

SEQ ID NO: 98 - RNA, 93 nt, synthetic construct
  source 1..93: mol_type = other RNA
atgacggaatataagctcgttgtggtaggggcttgtggagtgggcaagtccgcactgacaatccagctgatccagaatcactttgtggatgag

SEQ ID NO: 99 - RNA, 93 nt, synthetic construct
  source 1..93: mol_type = other RNA
atgaccgaatacaaacttgttgtggtgggcgctgacggagttggtaaaagcgcccttaccatccaactgattcagaaccactttgtcgacgag

SEQ ID NO: 100 - RNA, 93 nt, synthetic construct
  source 1..93: mol_type = other RNA
atgaccgagtataaattggtggtggtgggcgctggcgacgtgggcaaaagtgcactgacaattcagctgattcagaaccactttgtggacgag

SEQ ID NO: 101 - RNA, 93 nt, synthetic construct
  source 1..93: mol_type = other RNA
atgaccgagtataagttggtggttgtcggcgcggttggtgtgggaaaatctgcccttacaatccagctgattcaaaaccatttcgtcgacgag

SEQ ID NO: 102 - RNA, 93 nt, synthetic construct
  source 1..93: mol_type = other RNA
acgtgcttgctcgacatcctggatactgccggccacgaagagtactccgccatgcgggatcaatatatgcgtaccggcgaggggttcttatgt

SEQ ID NO: 103 - RNA, 93 nt, synthetic construct
  source 1..93: mol_type = other RNA
atgacagagtacaagctggtggtcgtcggagccggttgcgtaggaaagagcgcattaaccatccagctaatccaaaaccatttcgtggacgaa

SEQ ID NO: 104 - RNA, 93 nt, synthetic construct
  source 1..93: mol_type = other RNA
atgactgaatacaagttagtggtggttggggcctgtggtgtgggaaaatccgctctcacgatccagctgattcagaaccacttcgtcgacgag

SEQ ID NO: 105 - RNA, 93 nt, synthetic construct
  source 1..93: mol_type = other RNA
atgaccgaatataaactggttgtcgtgggggctgatggagttggcaagtctgccttgactattcaactgatccagaatcattttgtggatgag

SEQ ID NO: 106 - RNA, 93 nt, synthetic construct
  source 1..93: mol_type = other RNA
atgacagagtataaactcgtcgtggtgggtgccggagatgttgggaaatctgctcttaccatacagttaatccaaaatcacttcgttgatgaa

SEQ ID NO: 107 - RNA, 93 nt, synthetic construct
  source 1..93: mol_type = other RNA
atgactgagtacaagttggtggtggtgggggctgtgggggtgggaaagtctgcactgactattcagctcattcagaaccacttcgtggatgag

SEQ ID NO: 108 - RNA, 93 nt, synthetic construct
  source 1..93: mol_type = other RNA
acctgtctgctagacattctggacacggccggccatgaggagtatagcgcaatgagagaccaatacatgcggaccggcgaaggctttctttgc

SEQ ID NO: 109 - RNA, 531 nt, synthetic construct
  source 1..531: mol_type = other RNA
tgtataaacctcgactgggatgtgatccgcgataaaaccaagaccaagatcgaaagcctaaaggagcatggccctattaagaataaaatgtcagaatctcccaataaaacagtatccgaggaaaaggccaaacagtatttggaagagtttcaccagaccgccctggaacacccagagctgtccgaactgaagacagtcactgggacaaatcctgtcttcgccggtgctaactacgcggcatgggcggtcaatgtagcccaagttatcgatagtgagactgccgacaaccttgagaaaaccacagcagcattgagcatattgccgggcatcggttctgtcatgggcatagctgacggcgctgtgcaccacaacacagaggagatcgtcgctcagagtattgcgctgtccagtcttatggtggcccaggccatcccacttgttggtgaactggtggacatcggatttgcagcgtacaactttgtcgagtcaattattaatctgttccaggttgtccataacagttataatcgt

SEQ ID NO: 110 - RNA, 261 nt, synthetic construct
  source 1..261: mol_type = other RNA
tgtataaatcttgactgggacgtcattagggataagacaaagaccaagatagagtccttgaaggagcacggccccatcaaaaacaaaatgtcggagagcccaaacaaaacagtgagtgaagagaaagcaaagcagtatcttgaagagtttcaccagacggctctggaacatcctgagctgtctgaacttaagacagtgaccggcacaaaccctgtgtttgcaggagctaattatgcggcttgggccgttaacgtggctcag

SEQ ID NO: 111 - RNA, 93 nt, synthetic construct
  source 1..93: mol_type = other RNA
atgactgagtataaactggttgtggtgggcgccggctgcgtcggaaagagcgcgctgaccattcaacttattcaaaatcacttcgttgatgaa

SEQ ID NO: 112 - RNA, 93 nt, synthetic construct
  source 1..93: mol_type = other RNA
atgactgaatataagttagtggtcgtgggcgcctgtggggtgggtaaaagcgccctaactatacagctaatccagaaccactttgtagacgag

SEQ ID NO: 113 - RNA, 93 nt, synthetic construct
  source 1..93: mol_type = other RNA
atgacagagtacaagctggtggttgtaggtgcagacggcgtcggcaaatccgccctcactatccagctgattcagaaccactttgtagacgag

SEQ ID NO: 114 - RNA, 93 nt, synthetic construct
  source 1..93: mol_type = other RNA
atgactgaatacaaacttgttgtggttggggctggggatgtcggaaagagtgctctgacgattcagctgatccagaaccattttgtggacgaa

SEQ ID NO: 115 - RNA, 93 nt, synthetic construct
  source 1..93: mol_type = other RNA
atgaccgagtacaagctggtggttgtgggagcggtcggggtcgggaagtccgccttaacaatccagctgatccaaaatcacttcgtggatgaa

SEQ ID NO: 116 - RNA, 93 nt, synthetic construct
  source 1..93: mol_type = other RNA
acctgcctgctcgacatcctcgataccgccggtcacgaagagtactccgccatgcgtgatcaatatatgcgaacgggggagggtttcttatgc

SEQ ID NO: 117 - RNA, 246 nt, synthetic construct
  source 1..246: mol_type = other RNA
aatttggagaaaaccaccgcagcactatctatcctgccgggaataggtagcgtcatgggcatcgccgacggagcagtacatcataatactgaagaaattgtggcccaatctatcgccttaagttctttgatggtggctcaggctattcccctggtcggggagctggtagatattggcttcgctgcctacaacttcgtggagagcatcattaatttgttccaggtcgtgcataatagctacaacagg

SEQ ID NO: 118 - RNA, 51 nt, synthetic construct
  source 1..51: mol_type = other RNA
tacaagctagtggtggtcggcgcatgcggcgtgggaaagagcgcgctaacc

SEQ ID NO: 119 - RNA, 51 nt, synthetic construct
  source 1..51: mol_type = other RNA
tataaattggtcgttgtgggcgccgatggtgtagggaaatctgcgctgacc

SEQ ID NO: 120 - RNA, 51 nt, synthetic construct
  source 1..51: mol_type = other RNA
tacaagctcgtggtggtgggggcagtcggcgtcggcaaaagtgccctgact

SEQ ID NO: 121 - RNA, 51 nt, synthetic construct
  source 1..51: mol_type = other RNA
tataagctggtcgtggtaggtgctcgtggcgttggaaagtccgccttgaca

SEQ ID NO: 122 - RNA, 51 nt, synthetic construct
  source 1..51: mol_type = other RNA
aagctggttgtcgtgggggccggatgcgtgggcaaatctgctcttacaatc

SEQ ID NO: 123 - RNA, 51 nt, synthetic construct
  source 1..51: mol_type = other RNA
aaattagtagttgtgggagctggagacgtaggtaaatccgcactgacgata

SEQ ID NO: 124 - RNA, 51 nt, synthetic construct
  source 1..51: mol_type = other RNA
ctcgatattctcgacaccgctggacacgaagagtacagcgccatgcgcgac

SEQ ID NO: 125 - RNA, 51 nt, synthetic construct
  source 1..51: mol_type = other RNA
aagctggtcgtggtgggggccggcagagttggtaagtcagcactcactatc

SEQ ID NO: 126 - RNA, 93 nt, synthetic construct
  source 1..93: mol_type = other RNA
atgaccgagtacaaactcgttgtggtgggggcctgcggagtgggcaagtctgcattgaccatacagttgatccagaatcatttcgtggatgag

SEQ ID NO: 127 - RNA, 24 nt, synthetic construct
  source 1..24: mol_type = other RNA
gtcggagcgtgtggcgtgggcaaa

SEQ ID NO: 128 - RNA, 93 nt, synthetic construct
  source 1..93: mol_type = other RNA
atgactgagtataagttagtagtcgttggtgccgatggagtaggcaaatcggcgttaacgatccagcttattcagaatcatttcgtagatgag

SEQ ID NO: 129 - RNA, 24 nt, synthetic construct
  source 1..24: mol_type = other RNA
gtcggggccgacggcgtgggcaaa

SEQ ID NO: 130 - RNA, 93 nt, synthetic construct
  source 1..93: mol_type = other RNA
atgaccgagtataagttagtcgtcgtgggtgctgtgggcgtaggtaaatccgccctgactatacagcttattcaaaaccattttgtggatgag

SEQ ID NO: 131 - RNA, 24 nt, synthetic construct
  source 1..24: mol_type = other RNA
gtgggagctgtgggcgttgggaaa

SEQ ID NO: 132 - RNA, 93 nt, synthetic construct
  source 1..93: mol_type = other RNA
atgactgagtacaagctggtggtcgtcggtgcccggggcgttggtaagagcgccctgaccatacaactcattcagaaccactttgtagacgaa

SEQ ID NO: 133 - RNA, 24 nt, synthetic construct
  source 1..24: mol_type = other RNA
gtgggagcaaggggagtcgggaaa

SEQ ID NO: 134 - RNA, 93 nt, synthetic construct
  source 1..93: mol_type = other RNA
acagaatacaagctggttgtagtcggcgccggttgtgtcgggaagtccgcactcacaattcagcttatccagaatcacttcgtggacgagtat

SEQ ID NO: 135 - RNA, 24 nt, synthetic construct
  source 1..24: mol_type = other RNA
ggggcaggctgcgttggaaagagc

SEQ ID NO: 136 - RNA, 93 nt, synthetic construct
  source 1..93: mol_type = other RNA
acagagtacaaattagtggtcgtgggagctggggacgtgggaaagtccgctctcactatccagctgattcagaaccattttgtcgacgaatat

SEQ ID NO: 137 - RNA, 24 nt, synthetic construct
  source 1..24: mol_type = other RNA
ggggctggggacgtgggcaaatca

SEQ ID NO: 138 - RNA, 93 nt, synthetic construct
  source 1..93: mol_type = other RNA
acctgcctgctggatatcctggacacagcgggacacgaagaatacagcgccatgagagatcagtacatgcgaacgggtgaaggcttcctgtgc

SEQ ID NO: 139 - RNA, 24 nt, synthetic construct
  source 1..24: mol_type = other RNA
acagcagggcacgaggaatactcc

SEQ ID NO: 140 - RNA, 93 nt, synthetic construct
  source 1..93: mol_type = other RNA
actgaatataagctggtagttgttggcgcaggaagagttgggaagagtgctctaaccatccaacttatccagaaccactttgtggacgagtat

SEQ ID NO: 141 - RNA, 24 nt, synthetic construct
  source 1..24: mol_type = other RNA
ggcgccgggcgcgtcggaaagtct

SEQ ID NO: 142 - RNA, 93 nt, synthetic construct
  source 1..93: mol_type = other RNA
atgacagaatacaagttagtggtggtaggagcgtgtggagtggggaaaagcgccctaactattcagttgatacaaaaccatttcgtggacgaa

SEQ ID NO: 143 - RNA, 24 nt, synthetic construct
  source 1..24: mol_type = other RNA
gtgggcgcatgtggagtcggcaag

SEQ ID NO: 144 - RNA, 93 nt, synthetic construct
  source 1..93: mol_type = other RNA
atgaccgagtataagctcgtagtcgtgggggccgacggcgtggggaaatcagcccttactatccagcttatacaaaaccattttgttgacgaa

SEQ ID NO: 145 - RNA, 24 nt, synthetic construct
  source 1..24: mol_type = other RNA
gtaggcgccgacggggtcggaaag

SEQ ID NO: 146 - RNA, 93 nt, synthetic construct
  source 1..93: mol_type = other RNA
atgaccgagtacaagctggttgtcgtcggggccgtaggggttggcaaaagcgccctgactatccagctcatccaaaatcatttcgtcgacgag

SEQ ID NO: 147 - RNA, 24 nt, synthetic construct
  source 1..24: mol_type = other RNA
gtgggcgccgttggagtcggtaaa

SEQ ID NO: 148 - RNA, 93 nt, synthetic construct
  source 1..93: mol_type = other RNA
atgacggaatacaagttggtggtagtgggggctcggggggtgggcaagagtgctttgaccatacagttaattcagaaccactttgtggatgag

SEQ ID NO: 149 - RNA, 24 nt, synthetic construct
  source 1..24: mol_type = other RNA
gtcggcgctcgcggagtcggcaaa

SEQ ID NO: 150 - RNA, 93 nt, synthetic construct
  source 1..93: mol_type = other RNA
accgagtataaattggttgttgtcggcgcaggctgtgtgggcaagtctgctctcaccattcaactaatccagaaccactttgttgatgagtac

SEQ ID NO: 151 - RNA, 24 nt, synthetic construct
  source 1..24: mol_type = other RNA
ggagcaggctgcgtgggaaaaagc

SEQ ID NO: 152 - RNA, 93 nt, synthetic construct
  source 1..93: mol_type = other RNA
acggagtacaagctggttgtcgtaggagcaggagacgtgggcaaatccgctctgacaattcagcttatccagaatcactttgtggatgaatat

SEQ ID NO: 153 - RNA, 24 nt, synthetic construct
  source 1..24: mol_type = other RNA
ggtgccggggatgtcggtaaaagt

SEQ ID NO: 154 - RNA, 93 nt, synthetic construct
  source 1..93: mol_type = other RNA
acctgcttactggacatcctggacactgctggtcacgaggaatactccgcgatgcgagatcagtatatgcggacaggagagggtttcctgtgc

SEQ ID NO: 155 - RNA, 24 nt, synthetic construct
  source 1..24: mol_type = other RNA
acagcaggccatgaggaatattct

SEQ ID NO: 156 - RNA, 93 nt, synthetic construct
  source 1..93: mol_type = other RNA
actgaatataagctggtggtggtgggggctggtagagtggggaagtcagcactgacaatccagctcattcagaatcacttcgttgatgagtac

SEQ ID NO: 157 - RNA, 24 nt, synthetic construct
  source 1..24: mol_type = other RNA
ggtgcgggtagagtgggcaagtcc

SEQ ID NO: 158 - RNA, 93 nt, synthetic construct
  source 1..93: mol_type = other RNA
atgacggaatacaagctcgtcgtggtgggcgccgatggcgtcggcaagtcggcactgactatccagctcatacaaaatcattttgttgatgaa

SEQ ID NO: 159 - RNA, 24 nt, synthetic construct
  source 1..24: mol_type = other RNA
gtgggggcggacggagtggggaag

SEQ ID NO: 160 - RNA, 93 nt, synthetic construct
  source 1..93: mol_type = other RNA
atgaccgaatacaaactggtggtggtcggggcgtgtggggtgggcaaatcagccctcacaatacagctgattcagaaccactttgttgacgag

SEQ ID NO: 161 - RNA, 24 nt, synthetic construct
  source 1..24: mol_type = other RNA
gtgggggcatgtggagtggggaag

SEQ ID NO: 162 - RNA, 93 nt, synthetic construct
  source 1..93: mol_type = other RNA
atgactgagtacaagctggttgttgtaggtgctgttggagtgggaaagagcgccctcacaatccaattgatccagaatcacttcgtcgacgag

SEQ ID NO: 163 - RNA, 24 nt, synthetic construct
  source 1..24: mol_type = other RNA
gtgggggccgtaggcgtgggcaag

SEQ ID NO: 164 - RNA, 93 nt, synthetic construct
  source 1..93: mol_type = other RNA
atgaccgaatataagttggtagtggtgggggctcgcggagtcgggaagagtgctcttactattcagctgattcagaaccatttcgtagatgaa

SEQ ID NO: 165 - RNA, 24 nt, synthetic construct
  source 1..24: mol_type = other RNA
gtgggcgccagaggcgtgggcaaa

SEQ ID NO: 166 - RNA, 93 nt, synthetic construct
  source 1..93: mol_type = other RNA
actgagtacaaactagtagtcgtaggagctgggtgtgtgggcaaaagtgcactcaccatccagctgatccagaaccatttcgtggatgagtat

SEQ ID NO: 167 - RNA, 24 nt, synthetic construct
  source 1..24: mol_type = other RNA
ggagctggctgcgtgggcaaaagc

SEQ ID NO: 168 - RNA, 93 nt, synthetic construct
  source 1..93: mol_type = other RNA
acggaatacaaactagttgttgtgggggctggtgatgtcggaaagtccgccttgactattcagcttattcaaaatcactttgtcgacgagtat

SEQ ID NO: 169 - RNA, 24 nt, synthetic construct
  source 1..24: mol_type = other RNA
ggcgcaggtgacgtaggaaagtct

SEQ ID NO: 170 - RNA, 93 nt, synthetic construct
  source 1..93: mol_type = other RNA
acctgccttctggacatcctggacacagccggccacgaggaatacagcgccatgcgggatcagtatatgaggacaggagaaggcttcctctgc

SEQ ID NO: 171 - RNA, 24 nt, synthetic construct
  source 1..24: mol_type = other RNA
acagctggccacgaggagtattct

SEQ ID NO: 172 - RNA, 93 nt, synthetic construct
  source 1..93: mol_type = other RNA
accgagtacaaacttgtggtggttggagcagggcgtgtcggcaaatctgccctgaccatccagctgatacaaaaccattttgtggatgagtat

SEQ ID NO: 173 - RNA, 27 nt, synthetic construct
  source 1..27: mol_type = other RNA
ggcggtgcgggacgagtgggcaagagc

SEQ ID NO: 174 - RNA, 51 nt, synthetic construct
  source 1..51: mol_type = other RNA
tataagttggtggtggttggggcatgtggcgtaggtaagagcgcactgact

SEQ ID NO: 175 - RNA, 51 nt, synthetic construct
  source 1..51: mol_type = other RNA
tataagctagttgtcgtgggagctgacggagtggggaagagtgcactcaca

SEQ ID NO: 176 - RNA, 51 nt, synthetic construct
  source 1..51: mol_type = other RNA
tacaagctcgtcgtggtgggcgccgtcggcgtagggaaatcagctcttacc

SEQ ID NO: 177 - RNA, 51 nt, synthetic construct
  source 1..51: mol_type = other RNA
tataaactcgtggtggtgggcgctcgcggtgtgggcaaaagtgccctgacc

SEQ ID NO: 178 - RNA, 51 nt, synthetic construct
  source 1..51: mol_type = other RNA
aagctggtcgtcgtgggcgccggctgcgttgggaaatccgccctgacaatc

SEQ ID NO: 179 - RNA, 51 nt, synthetic construct
  source 1..51: mol_type = other RNA
aaattggtcgtcgtgggcgccggggacgtgggtaagtcagccttgaccatc

SEQ ID NO: 180 - RNA, 51 nt, synthetic construct
  source 1..51: mol_type = other RNA
aaattagtggtggtaggtgccggtagagtggggaagtcggctctcaccatt

SEQ ID NO: 181 - RNA, 51 nt, synthetic construct
  source 1..51: mol_type = other RNA
ctcgacatactggacactgccggccacgaagaatattctgccatgagggat

SEQ ID NO: 182 - RNA, 51 nt, synthetic construct
  source 1..51: mol_type = other RNA
tacaagctggtggtagtcggcgcatgtggagtgggaaaaagcgccctgaca

SEQ ID NO: 183 - RNA, 51 nt, synthetic construct
  source 1..51: mol_type = other RNA
tacaaattagtggtggtcggtgctgatggggtcggcaagtcagctctgacc

SEQ ID NO: 184 - RNA, 51 nt, synthetic construct
  source 1..51: mol_type = other RNA
tacaaactcgttgtcgttggggccgtcggagtgggtaaatccgccctgacc

SEQ ID NO: 185 - RNA, 51 nt, synthetic construct
  source 1..51: mol_type = other RNA
tataaattggtcgtggtaggggctcggggagtcgggaaatctgcgctgacc

SEQ ID NO: 186 - RNA, 51 nt, synthetic construct
  source 1..51: mol_type = other RNA
aagctcgttgtggtgggagcaggttgcgttggtaagtctgctttgactatc

SEQ ID NO: 187 - RNA, 51 nt, synthetic construct
  source 1..51: mol_type = other RNA
ctcgacatcctcgacaccgcaggacatgaggagtactccgccatgcgtgac

SEQ ID NO: 188 - RNA, 51 nt, synthetic construct
  source 1..51: mol_type = other RNA
aagctagtagttgtaggagccggtgatgtgggaaagtccgctctgacaatt

SEQ ID NO: 189 - RNA, 51 nt, synthetic construct
  source 1..51: mol_type = other RNA
aaattggtagtcgtgggggccggcagagtgggaaaaagcgcgctgacaatc

SEQ ID NO: 190 - RNA, 51 nt, synthetic construct
  source 1..51: mol_type = other RNA
tataaacttgttgtggtgggcgcagtcggagttggtaagtcagctctgact

SEQ ID NO: 191 - RNA, 51 nt, synthetic construct
  source 1..51: mol_type = other RNA
tacaagctcgttgttgtgggcgctcgcggggtgggcaagtctgccctaaca

SEQ ID NO: 192 - RNA, 51 nt, synthetic construct
  source 1..51: mol_type = other RNA
aaactagtggtggtcggcgccgggtgcgtaggaaagagtgctctgaccata

SEQ ID NO: 193 - RNA, 51 nt, synthetic construct
  source 1..51: mol_type = other RNA
cttgacattctggatactgcgggccacgaggaatatagcgcgatgcgagat

SEQ ID NO: 194 - RNA, 51 nt, synthetic construct
  source 1..51: mol_type = other RNA
tacaaactggttgttgtgggagcatgtggcgtcgggaagtccgcattaacg

SEQ ID NO: 195 - RNA, 51 nt, synthetic construct
  source 1..51: mol_type = other RNA
tacaagttagtggtggtcggagcggatggcgttgggaagtctgcacttacg

SEQ ID NO: 196 - RNA, 51 nt, synthetic construct
  source 1..51: mol_type = other RNA
aagttggtggtggtgggggccggagacgtaggcaagtccgcacttaccatc

SEQ ID NO: 197 - RNA, 51 nt, synthetic construct
  source 1..51: mol_type = other RNA
aaactggttgtcgtcggcgccgggcgggtgggcaagagcgcactgactatt

SEQ ID NO: 198 - RNA, 93 nt, synthetic construct; identical to SEQ ID NO: 158
  source 1..93: mol_type = other RNA

SEQ ID NO: 199 - RNA, 24 nt, synthetic construct
  source 1..24: mol_type = other RNA
gtgggggcggacggagtggggaag

SEQ ID NO: 200 - RNA, 93 nt, synthetic construct; identical to SEQ ID NO: 160
  source 1..93: mol_type = other RNA

SEQ ID NO: 201 - RNA, 24 nt, synthetic construct
  source 1..24: mol_type = other RNA
gtgggggcatgtggagtggggaag

SEQ ID NO: 202 - RNA, 93 nt, synthetic construct; identical to SEQ ID NO: 162
  source 1..93: mol_type = other RNA

SEQ ID NO: 203 - RNA, 24 nt, synthetic construct
  source 1..24: mol_type = other RNA
gtgggggccgtaggcgtgggcaag

SEQ ID NO: 204 - RNA, 93 nt, synthetic construct; identical to SEQ ID NO: 164
  source 1..93: mol_type = other RNA

SEQ ID NO: 205 - RNA, 24 nt, synthetic construct
  source 1..24: mol_type = other RNA
gtgggcgccagaggcgtgggcaaa

SEQ ID NO: 206 - RNA, 93 nt, synthetic construct; identical to SEQ ID NO: 166
  source 1..93: mol_type = other RNA

SEQ ID NO: 207 - RNA, 24 nt, synthetic construct
  source 1..24: mol_type = other RNA
ggagctggctgcgtgggcaaaagc

SEQ ID NO: 208 - RNA, 93 nt, synthetic construct; identical to SEQ ID NO: 168
  source 1..93: mol_type = other RNA

SEQ ID NO: 209 - RNA, 24 nt, synthetic construct
  source 1..24: mol_type = other RNA
ggcgcaggtgacgtaggaaagtct

SEQ ID NO: 210 - RNA, 93 nt, synthetic construct; identical to SEQ ID NO: 170
  source 1..93: mol_type = other RNA

SEQ ID NO: 211 - RNA, 24 nt, synthetic construct
  source 1..24: mol_type = other RNA
acagctggccacgaggagtattct

SEQ ID NO: 212 - RNA, 93 nt, synthetic construct
  source 1..93: mol_type = other RNA
atgacagagtataaactcgtggtggtcggcgcagatggcgttggaaagagcgccctgactattcagctcatccagaaccatttcgtagacgaa

SEQ ID NO: 213 - RNA, 24 nt, synthetic construct
  source 1..24: mol_type = other RNA
gtcggcgcagacggtgtgggaaag

SEQ ID NO: 214 - RNA, 93 nt, synthetic construct
  source 1..93: mol_type = other RNA
atgaccgaatacaagttagtggtggtcggggcctgcggtgtgggtaaaagcgctctgaccatacagcttatccagaaccacttcgtggatgag

SEQ ID NO: 215 - RNA, 24 nt, synthetic construct
  source 1..24: mol_type = other RNA
gtgggggcttgcggagttggcaag

SEQ ID NO: 216 - RNA, 93 nt, synthetic construct
  source 1..93: mol_type = other RNA
atgacagaatataaattggtggtagtgggggccgtaggcgtcggcaagagcgccctgactatacaactgatccaaaaccacttcgtggacgag

SEQ ID NO: 217 - RNA, 24 nt, synthetic construct
  source 1..24: mol_type = other RNA
gtgggcgccgttggagtcgggaaa

SEQ ID NO: 218 - RNA, 93 nt, synthetic construct
  source 1..93: mol_type = other RNA
atgactgagtataaacttgttgtagtcggggcacggggcgtgggaaagtccgctttaaccatacaattgatacagaaccactttgttgatgaa

SEQ ID NO: 219 - RNA, 24 nt, synthetic construct
  source 1..24: mol_type = other RNA
gtgggtgccaggggagtcggtaag

SEQ ID NO: 220 - RNA, 93 nt, synthetic construct
  source 1..93: mol_type = other RNA
acctgtctcctagacattctggacacagccggccacgaggagtactcagctatgcgcgatcagtatatgagaacaggggagggatttctgtgt

SEQ ID NO: 221 - RNA, 24 nt, synthetic construct
  source 1..24: mol_type = other RNA
acagcagggcacgaagagtacagc

SEQ ID NO: 222 - RNA, 93 nt, synthetic construct
  source 1..93: mol_type = other RNA
actgagtacaagctagtcgtggttggcgcaggcgacgtgggtaaatctgccctcaccattcagttaattcagaatcatttcgtcgatgaatat

SEQ ID NO: 223 - RNA, 24 nt, synthetic construct
  source 1..24: mol_type = other RNA
ggcgctggagacgtcggcaaatcc

SEQ ID NO: 224 - RNA, 93 nt, synthetic construct
  source 1..93: mol_type = other RNA
atgacggagtataaactggtagtcgtaggtgctgccggcgtcggcaaaagcgcgctaaccatccagctgatccagaatcactttgtggatgaa

SEQ ID NO: 225 - RNA, 24 nt, synthetic construct
  source 1..24: mol_type = other RNA
gtgggagccgctggagtggggaag

SEQ ID NO: 226 - RNA, 93 nt, synthetic construct
  source 1..93: mol_type = other RNA
agatcttacggaatccctttcatcgaaacctcaaccaagactcgtcaaagggttgaagacgctttctatacacttgtcagagagattaggcag

SEQ ID NO: 227 - RNA, 24 nt, synthetic construct
  source 1..24: mol_type = other RNA
gaaacctccacaaagacgcggcaa

SEQ ID NO: 228 - RNA, 93 nt, synthetic construct
  source 1..93: mol_type = other RNA
actgagtacaagctggtggtagtgggagccggttgcgtgggcaagtcagcgctgacaatccagctcatccagaatcattttgttgatgagtac

SEQ ID NO: 229 - RNA, 24 nt, synthetic construct
  source 1..24: mol_type = other RNA
ggcgcaggctgcgtggggaaatcg

SEQ ID NO: 230 - RNA, 93 nt, synthetic construct
  source 1..93: mol_type = other RNA
atgactgagtacaagctggtggtggtgggggcgtctggggtggggaaatctgcactgacgattcaactgatccagaatcattttgtagacgag

SEQ ID NO: 231 - RNA, 24 nt, synthetic construct
  source 1..24: mol_type = other RNA
gtgggcgcgtccggcgtgggtaag

SEQ ID NO: 232 - RNA, 93 nt, synthetic construct
  source 1..93: mol_type = other RNA
acctgtttgctggatattctggacactgccggacgagaagaatacagtgcaatgagggatcagtatatgcgaaccggagagggctttctttgt

SEQ ID NO: 233 - RNA, 24 nt, synthetic construct
  source 1..24: mol_type = other RNA
acagccggccgcgaagagtacagc

SEQ ID NO: 234 - RNA, 93 nt, synthetic construct
  source 1..93: mol_type = other RNA
atgacggaatataagctcgtggtcgttggggcctgtggcgtgggtaagagcgcactaaccattcagttaatccagaaccactttgttgatgaa

SEQ ID NO: 235 - RNA, 24 nt, synthetic construct
  source 1..24: mol_type = other RNA
gttggggcctgcggcgttggaaaa

SEQ ID NO: 236 - RNA, 93 nt, synthetic construct
  source 1..93: mol_type = other RNA
atgacagagtacaaactggtcgtggtgggggctgtgggtgtgggcaagtccgccctgactatccaactgatacagaaccatttcgtagatgaa

SEQ ID NO: 237 - RNA, 24 nt, synthetic construct
  source 1..24: mol_type = other RNA
gtcggcgcagtcggggtaggtaaa

SEQ ID NO: 238 - RNA, 93 nt, synthetic construct
  source 1..93: mol_type = other RNA
atgactgagtataaactcgtggttgtgggtgcagatggagtaggaaagagtgccctcacaatacaactgattcagaatcattttgtcgatgaa

SEQ ID NO: 239 - RNA, 24 nt, synthetic construct
  source 1..24: mol_type = other RNA
gtgggagctgatggtgttggcaag

SEQ ID NO: 240 - RNA, 93 nt, synthetic construct
  source 1..93: mol_type = other RNA
acctgcctgctggacatcctggacaccgcggggcacgaagagtatagcgctatgcgggaccagtacatgagaaccggtgagggctttctgtgt

SEQ ID NO: 241 - RNA, 24 nt, synthetic construct
  source 1..24: mol_type = other RNA
acagccggccacgaggagtattct

SEQ ID NO: 242 - RNA, 93 nt, synthetic construct
  source 1..93: mol_type = other RNA
actgagtacaagctggtggtggttggcgctggctgtgtggggaaatctgcattaacgattcaactgatccagaatcacttcgtcgatgagtac

SEQ ID NO: 243 - RNA, 24 nt, synthetic construct
  source 1..24: mol_type = other RNA
ggcgcaggctgcgtgggcaagtca

SEQ ID NO: 244 - RNA, 93 nt, synthetic construct
  source 1..93: mol_type = other RNA
acagagtacaagctggttgtcgttggggcaggggacgtcgggaagtccgccctgactatccagctgattcagaaccattttgtggacgaatat

SEQ ID NO: 245 - RNA, 24 nt, synthetic construct
  source 1..24: mol_type = other RNA
ggtgctggagacgtgggaaaatcc

SEQ ID NO: 246 - RNA, 93 nt, synthetic construct
  source 1..93: mol_type = other RNA
atgacagagtacaaacttgtggtggttggcgctgctggagtaggaaaatccgccctgaccatacaacttattcagaatcacttcgtggacgag

SEQ ID NO: 247 - RNA, 24 nt, synthetic construct
  source 1..24: mol_type = other RNA
gtcggggcggccggggtggggaag

SEQ ID NO: 248 - RNA, 93 nt, synthetic construct
  source 1..93: mol_type = other RNA
atgactgaatacaagttggtggtggtgggcgcgagcggggtgggtaagtctgccttgaccatccagctgatccagaaccatttcgttgatgaa

SEQ ID NO: 249 - RNA, 24 nt, synthetic construct
  source 1..24: mol_type = other RNA
gtcggcgcctcgggcgtggggaaa

SEQ ID NO: 250 - RNA, 93 nt, synthetic construct
  source 1..93: mol_type = other RNA
atgacagagtacaaactggtagtagtgggagcagatggggttggcaaatccgcgctcaccatccagctgattcaaaaccatttcgtcgatgag

SEQ ID NO: 251 - RNA, 24 nt, synthetic construct
  source 1..24: mol_type = other RNA
gtgggcgctgacggggtgggcaag

SEQ ID NO: 252 - RNA, 93 nt, synthetic construct
  source 1..93: mol_type = other RNA
atgaccgagtataaactcgtagtggtcggagctgtgggagtgggaaagtcagcgctcactatccaactaatacagaatcactttgtcgacgaa

SEQ ID NO: 253 - RNA, 24 nt, synthetic construct
  source 1..24: mol_type = other RNA
gtgggcgccgtgggtgtcggcaag

SEQ ID NO: 254 - RNA, 93 nt, synthetic construct
  source 1..93: mol_type = other RNA
atgacagaatacaagttggtcgttgttggtgcccgcggagtgggcaaatctgcactgaccatccaactgatccagaaccactttgtggatgag

SEQ ID NO: 255 - RNA, 24 nt, synthetic construct
  source 1..24: mol_type = other RNA
gtcggtgccaggggtgtgggaaag

SEQ ID NO: 256 - RNA, 93 nt, synthetic construct
  source 1..93: mol_type = other RNA
acatgtttgttagacattctcgacacggctgggcatgaggagtatagcgctatgcgggatcagtatatgagaaccggcgaaggctttctctgc

SEQ ID NO: 257 - RNA, 24 nt, synthetic construct
  source 1..24: mol_type = other RNA
acagccgggcacgaggaatattcc

SEQ ID NO: 258 - RNA, 93 nt, synthetic construct
  source 1..93: mol_type = other RNA
acttgcttgctggacatattagacacagcagggagggaagagtattccgcaatgcgagatcagtacatgcgtactggggaagggttcctgtgc

SEQ ID NO: 259 - RNA, 24 nt, synthetic construct
  source 1..24: mol_type = other RNA
accgccgggagggaggaatacagc

SEQ ID NO: 260 - RNA, 93 nt, synthetic construct
  source 1..93: mol_type = other RNA
actgagtacaagctggttgtggtgggagccggatgtgtcggtaaaagtgccctaaccatccagctcattcagaaccacttcgtcgatgagtat

SEQ ID NO: 261 - RNA, 93 nt, synthetic construct
  source 1..93: mol_type = other RNA
atgactgagtacaagctcgttgtcgtgggtgcatgcggagtagggaagtcagcgctgacaattcagttaatccagaatcacttcgtcgatgaa

SEQ ID NO: 262 - RNA, 24 nt, synthetic construct
  source 1..24: mol_type = other RNA
ggggccggctgtgttgggaaaagc

SEQ ID NO: 263 - RNA, 24 nt, synthetic construct
  source 1..24: mol_type = other RNA
gtcggcgcctgtggagtgggtaag

SEQ ID NO: 264 - RNA, 93 nt, synthetic construct
  source 1..93: mol_type = other RNA
acggagtacaagctggtcgtggttggcgccggcgacgtgggcaaaagcgctttgaccattcaactgatccagaatcattttgtggatgaatac

SEQ ID NO: 265 - RNA, 24 nt, synthetic construct
  source 1..24: mol_type = other RNA
ggggcaggggacgtggggaaatct

SEQ ID NO: 266 - RNA, 93 nt, synthetic construct
  source 1..93: mol_type = other RNA
atgactgaatataaattagttgtggtgggagccgatggagtggggaaatccgcgttgacaatccagctgatccagaaccattttgttgacgaa

SEQ ID NO: 267 - RNA, 24 nt, synthetic construct
  source 1..24: mol_type = other RNA
gttggagctgatggcgttggcaag

SEQ ID NO: 268 - RNA, 93 nt, synthetic construct
  source 1..93: mol_type = other RNA
atgacggaatacaagctggtcgtcgtgggcgcggtcggggtaggtaagtccgcgctgacaattcagctgattcagaatcacttcgtggatgag

SEQ ID NO: 269 - RNA, 24 nt, synthetic construct
  source 1..24: mol_type = other RNA
gtgggcgccgtgggagtcggtaag

SEQ ID NO: 270 - RNA, 93 nt, synthetic construct
  source 1..93: mol_type = other RNA
accgagtacaagctcgtggttgtcggtgctggtgacgtgggcaagtcggctctgacgatccaactgatccagaatcatttcgttgatgagtac

SEQ ID NO: 271 - RNA, 24 nt, synthetic construct
  source 1..24: mol_type = other RNA
ggggccggagatgtcggcaaatct

SEQ ID NO: 272 - RNA, 93 nt, synthetic construct
  source 1..93: mol_type = other RNA
aggtcatacgggatacctttcattgagacttccaccaaaactcgtcaacgcgtcgaggatgccttttataccctagtgagagagatccgacag

SEQ ID NO: 273 - RNA, 24 nt, synthetic construct
  source 1..24: mol_type = other RNA
gaaacaagcaccaagacccggcag

SEQ ID NO: 274 - RNA, 93 nt, synthetic construct
  source 1..93: mol_type = other RNA
atgacagaatacaagttggtagttgtcggcgcctgcggggtggggaaaagtgctctaaccattcaattaatccaaaatcactttgtcgacgag

SEQ ID NO: 275 - RNA, 24 nt, synthetic construct
  source 1..24: mol_type = other RNA
gtgggcgcctgcggggtaggcaaa

SEQ ID NO: 276 - RNA, 93 nt, synthetic construct
  source 1..93: mol_type = other RNA
atgaccgaatataaactggtggtggtgggagctgcaggagttggaaaaagcgctctgactatccaactgattcagaaccatttcgtggacgag

SEQ ID NO: 277 - RNA, 24 nt, synthetic construct
  source 1..24: mol_type = other RNA
gtcggggcagccggcgtgggaaaa

SEQ ID NO: 278 - RNA, 93 nt, synthetic construct
  source 1..93: mol_type = other RNA
atgaccgaatacaagctggtggtggttggcgcatccggtgtgggaaagtcagccctgacaatccagcttatacagaaccacttcgtcgacgaa

SEQ ID NO: 279 - RNA, 24 nt, synthetic construct
  source 1..24: mol_type = other RNA
gtgggcgcatctggagtcggaaag

SEQ ID NO: 280 - RNA, 93 nt, synthetic construct
  source 1..93: mol_type = other RNA
acagagtataaattggtggtggtaggtgctggatgtgtcggcaagtccgcacttactatacagctcattcagaaccactttgtagacgagtat

SEQ ID NO: 281 - RNA, 24 nt, synthetic construct
  source 1..24: mol_type = other RNA
ggggcagggtgcgtaggcaagtcc

SEQ ID NO: 282 - RNA, 93 nt, synthetic construct
  source 1..93: mol_type = other RNA
atgacggaatataagttggtcatggtgggtgcggatggtgtaggcaaatcagctctgactatccaactgatccagaatcactttgtggacgaa

SEQ ID NO: 283 - RNA, 24 nt, synthetic construct
  source 1..24: mol_type = other RNA
gtcggggctgacggtgtcggaaaa

SEQ ID NO: 284 - RNA, 93 nt, synthetic construct
  source 1..93: mol_type = other RNA
atgaccgaatataagctcgttgtggtgggggcttgtggagttggcaagtccgctctgactatccaactaattcagaatcatttcgtcgatgag

SEQ ID NO: 285 - RNA, 24 nt, synthetic construct
  source 1..24: mol_type = other RNA
gtcggcgcttgcggagtaggcaaa

SEQ ID NO: 286 - RNA, 93 nt, synthetic construct
  source 1..93: mol_type = other RNA
atgaccgaatacaagctcgtggtggtaggcgccgttggtgtagggaagagtgcgctaaccattcaacttatccagaaccattttgttgacgag

SEQ ID NO: 287 - RNA, 24 nt, synthetic construct
  source 1..24: mol_type = other RNA
gtaggcgcggtgggagtgggcaag

SEQ ID NO: 288 - RNA, 93 nt, synthetic construct
  source 1..93: mol_type = other RNA
atgaccgagtataaacttgtagtggtgggcgccaggggcgtggggaaaagcgccctgacaatccagctcatacagaatcacttcgtagacgag

SEQ ID NO: 289 - RNA, 24 nt, synthetic construct
  source 1..24: mol_type = other RNA
gtgggcgcccgcggcgttggtaag

SEQ ID NO: 290 - RNA, 93 nt, synthetic construct
  source 1..93: mol_type = other RNA
actgaatataaactcgtcgtcgtgggagcaggctgtgtggggaagagtgccttgaccattcagttgatccagaaccacttcgtcgacgagtat

SEQ ID NO: 291 - RNA, 24 nt, synthetic construct
  source 1..24: mol_type = other RNA
ggagcagggtgcgttggaaagagc

SEQ ID NO: 292 - RNA, 93 nt, synthetic construct
  source 1..93: mol_type = other RNA
acagaatacaaactggttgtcgtgggggccggcgacgtgggcaagagcgccttgactattcaactcatacagaaccactttgtggatgagtac

SEQ ID NO: 293 - RNA, 24 nt, synthetic construct
  source 1..24: mol_type = other RNA
ggcgccggggacgtggggaaaagc

SEQ ID NO: 294 - RNA, 93 nt, synthetic construct
  source 1..93: mol_type = other RNA
acgtgtcttctggatattttagacaccgcagggcatgaggagtactccgcaatgcgagatcagtacatgaggacaggtgagggtttcctgtgc

SEQ ID NO: 295 - RNA, 24 nt, synthetic construct
  source 1..24: mol_type = other RNA
acagccggccacgaggaatactct

SEQ ID NO: 296 - RNA, 797 nt, synthetic construct
  source 1..797: mol_type = other RNA
aagctttaatacgactcactataagtcctccccatcctctccctctgtccctctgtccctctgaccctgcactgtcccagcaccatggatatgagggtgcccgcccagctgctggggctgctgctgctgtggctgcggggcgcccggtgctacaagctggtcgtcgtgggggcctgcggcgtggggaagtccgctctgacctacaaactggtggtggtgggggccgatggggtgggcaagtccgccctgacctacaagctggtggtggtcggggccgtcggcgtgggcaaaagcgccctgacatacaagctcgtggtggtgggcgccaggggggtgggcaagtccgccctgacaaagctggtcgtggtgggcgccgggtgcgtgggcaagagcgccctgacaatcaagctggtggtggtgggggccggggatgtgggcaagagcgccctgaccatcctggacatcctggacacagccgggcacgaggagtacagcgccatgagggattgatgactcgaggtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattattttaactatgctagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgaattc

SEQ ID NO: 297 - RNA, 881 nt, synthetic construct
  source 1..881: mol_type = other RNA
aagctttaatacgactcactataagtcctccccatcctctccctctgtccctctgtccctctgaccctgcactgtcccagcaccatggatatgagagtgcccgcccaactgctggggctgctgctgctgtggctgagaggcgcgagatgcatgacagaatacaaacttgtggtagttggagcatgcggtgtgggtaagagtgccctcaccatccaactaattcagaaccacttcgttgacgaactcctctcagtggggggggttggagcatgtggtgtagggaagctgttgagcgtcggcgggatgactgaatacaaacttgtggttgttggagcagatggggtcggaaaatccgccttgacaatacagcttattcagaatcactttgtggatgagctgttaagcgttggcggcgtcggagctgatggcgtcggaaagctcttgtctgtgggcggcactgagtataaattagtggtagtcggggctggagacgtgggcaagagtgcccttaccatccagctcatccagaatcattttgtggacgagtatctattgtccgtgggaggtggagctggtgacgtcggaaagtcttaatgactcgaggtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattattttaactatgctagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgaattc

SEQ ID NO: 298 - RNA, 1365 nt, synthetic construct
  source 1..1365: mol_type = other RNA
aagctttaatacgactcactataagaatccagtcaacgcatatccacgtatacccgaacacatcacagccaccatggatatgcgagtgcccgctcagcttctggggttgcttctgttatggctgaggggggcccggtgtatgacagaatacaagttagtggtggtaggagcgtgtggagtggggaaaagcgccctaactattcagttgatacaaaaccatttcgtggacgaagtgggcgcatgtggagtcggcaagatgaccgagtataagctcgtagtcgtgggggccgacggcgtggggaaatcagcccttactatccagcttatacaaaaccattttgttgacgaagtaggcgccgacggggtcggaaagatgaccgagtacaagctggttgtcgtcggggccgtaggggttggcaaaagcgccctgactatccagctcatccaaaatcatttcgtcgacgaggtgggcgccgttggagtcggtaaaatgacggaatacaagttggtggtagtgggggctcggggggtgggcaagagtgctttgaccatacagttaattcagaaccactttgtggatgaggtcggcgctcgcggagtcggcaaaaccgagtataaattggttgttgtcggcgcaggctgtgtgggcaagtctgctctcaccattcaactaatccagaaccactttgttgatgagtacggagcaggctgcgtgggaaaaagcacggagtacaagctggttgtcgtaggagcaggagacgtgggcaaatccgctctgacaattcagcttatccagaatcactttgtggatgaatatggtgccggggatgtcggtaaaagtacctgcttactggacatcctggacactgctggtcacgaggaatactccgcgatgcgagatcagtatatgcggacaggagagggtttcctgtgcacagcaggccatgaggaatattctactgaatataagctggtggtggtgggggctggtagagtggggaagtcagcactgacaatccagctcattcagaatcacttcgttgatgagtacggtgcgggtagagtgggcaagtcctaatgactcgaggtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattattttaactatgctagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgaattc

SEQ ID NO: 299 - RNA, 1635 nt, synthetic construct
  source 1..1635: mol_type = other RNA
aagctttaatacgactcactataagaatccagtcaacgcatatccacgtatacccgaacacatcacagccaccatggatatgcgagtgcccgctcagcttctggggttgcttctgttatggctgaggggggcccggtgtatgacggaatacaagctcgtcgtggtgggcgccgatggcgtcggcaagtcggcactgactatccagctcatacaaaatcattttgttgatgaacttttatccgtcggtggagtgggggcggacggagtggggaagctgctgagcgttggtgggatgaccgaatacaaactggtggtggtcggggcgtgtggggtgggcaaatcagccctcacaatacagctgattcagaaccactttgttgacgagcttctgtccgtgggcggggtgggggcatgtggagtggggaagctcctgtccgtagggggcatgactgagtacaagctggttgttgtaggtgctgttggagtgggaaagagcgccctcacaatccaattgatccagaatcacttcgtcgacgagctcttgagcgttggaggtgtgggggccgtaggcgtgggcaagctgctatctgtgggtggaatgaccgaatataagttggtagtggtgggggctcgcggagtcgggaagagtgctcttactattcagctgattcagaaccatttcgtagatgaactcctatcagtcggtggcgtgggcgccagaggcgtgggcaaactgttatcagtgggcggtactgagtacaaactagtagtcgtaggagctgggtgtgtgggcaaaagtgcactcaccatccagctgatccagaaccatttcgtggatgagtatttgttgagtgtcggaggtggagctggctgcgtgggcaaaagcctgctgtccgttggggggacggaatacaaactagttgttgtgggggctggtgatgtcggaaagtccgccttgactattcagcttattcaaaatcactttgtcgacgagtatcttctgagcgtcggcggcggcgcaggtgacgtaggaaagtctttactgtcagttgggggaacctgccttctggacatcctggacacagccggccacgaggaatacagcgccatgcgggatcagtatatgaggacaggagaaggcttcctctgcctcttaagtgtcggcggtacagctggccacgaggagtattctctgctgtctgtcggaggcaccgagtacaaacttgtggtggttggagcagggcgtgtcggcaaatctgccctgaccatccagctgatacaaaaccattttgtggatgagtatttactttccgtgggaggcggtgcgggacgagtgggcaagagctaatgactcgaggtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattattttaactatgctagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgaattc

SEQ ID NO: 300 - RNA, 1482 nt, synthetic construct
  source 1..1482: mol_type = other RNA
aagctttaatacgactcactataagaatccagtcaacgcatatccacgtatacccgaacacatcacagccaccatggacatgagggtgcccgcccagttgctggggctgctgctcttgtggctcagaggggcacggtgcatgacggaatacaagctcgtcgtggtgggcgccgatggcgtcggcaagtcggcactgactatccagctcatacaaaatcattttgttgatgaacttttatccgtcggtggagtgggggcggacggagtggggaagctgctgagcgttggtgggatgaccgaatacaaactggtggtggtcggggcgtgtggggtgggcaaatcagccctcacaatacagctgattcagaaccactttgttgacgagcttctgtccgtgggcggggtgggggcatgtggagtggggaagctcctgtccgtagggggcatgactgagtacaagctggttgttgtaggtgctgttggagtgggaaagagcgccctcacaatccaattgatccagaatcacttcgtcgacgagctcttgagcgttggaggtgtgggggccgtaggcgtgggcaagctgctatctgtgggtggaatgaccgaatataagttggtagtggtgggggctcgcggagtcgggaagagtgctcttactattcagctgattcagaaccatttcgtagatgaactcctatcagtcggtggcgtgggcgccagaggcgtgggcaaactgttatcagtgggcggtactgagtacaaactagtagtcgtaggagctgggtgtgtgggcaaaagtgcactcaccatccagctgatccagaaccatttcgtggatgagtatttgttgagtgtcggaggtggagctggctgcgtgggcaaaagcctgctgtccgttggggggacggaatacaaactagttgttgtgggggctggtgatgtcggaaagtccgccttgactattcagcttattcaaaatcactttgtcgacgagtatcttctgagcgtcggcggcggcgcaggtgacgtaggaaagtctttactgtcagttgggggaacctgccttctggacatcctggacacagccggccacgaggaatacagcgccatgcgggatcagtatatgaggacaggagaaggcttcctctgcctcttaagtgtcggcggtacagctggccacgaggagtattcttaatgactcgaggtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattattttaactatgctagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgaattc

SEQ ID NO: 301 - protein, 23 aa, synthetic construct
MTEYKLVVVGARGVGKSALTIQL

SEQ ID NO: 302 - protein, 19 aa, synthetic construct
EYKLVVVGADGVGKSALTI

SEQ ID NO: 303 - protein, 19 aa, synthetic construct
EYKLVVVGAVGVGKSALTI

SEQ ID NO: 304 - protein, 19 aa, synthetic construct
EYKLVVVGACGVGKSALTI

SEQ ID NO: 305 - protein, 19 aa, synthetic construct
YKLVVVGAGDVGKSALTIQ

SEQ ID NO: 306 - protein, 19 aa, synthetic construct
YKLVVVGAGCVGKSALTIQ

SEQ ID NO: 307 - protein, 19 aa, synthetic construct
LLDILDTAGHEEYSAMRDQ

SEQ ID NO: 308 - protein, 17 aa, synthetic construct
YKLVVVGACGVGKSALT

SEQ ID NO: 309 - protein, 17 aa, synthetic construct
YKLVVVGADGVGKSALT

SEQ ID NO: 310 - protein, 17 aa, synthetic construct
YKLVVVGAVGVGKSALT

SEQ ID NO: 311 - protein, 17 aa, synthetic construct
YKLVVVGARGVGKSALT

SEQ ID NO: 312 - protein, 17 aa, synthetic construct
KLVVVGAGCVGKSALTI

SEQ ID NO: 313 - protein, 17 aa, synthetic construct
KLVVVGAGDVGKSALTI

SEQ ID NO: 314 - protein, 17 aa, synthetic construct
LDILDTAGHEEYSAMRD

SEQ ID NO: 315 - protein, 17 aa, synthetic construct
YKLVVVGACGVGKSALT

SEQ ID NO: 316 - protein, 17 aa, synthetic construct
YKLVVVGADGVGKSALT

SEQ ID NO: 317 - protein, 17 aa, synthetic construct
YKLVVVGAVGVGKSALT

SEQ ID NO: 318 - protein, 17 aa, synthetic construct
YKLVVVGARGVGKSALT

SEQ ID NO: 319 - protein, 17 aa, synthetic construct
KLVVVGAGCVGKSALTI

SEQ ID NO: 320 - protein, 17 aa, synthetic construct
KLVVVGAGDVGKSALTI

SEQ ID NO: 321 - protein, 17 aa, synthetic construct
LDILDTAGHEEYSAMRD

SEQ ID NO: 322 - protein, 9 aa, synthetic construct
VGACGVGKS

SEQ ID NO: 323 - protein, 9 aa, synthetic construct
VVVGACGVG

SEQ ID NO: 324 - protein, 9 aa, synthetic construct
VGACGVGKS

SEQ ID NO: 325 - protein, 9 aa, synthetic construct
VVVGACGVG

SEQ ID NO: 326 - protein, 9 aa, synthetic construct
VGACGVGKS

SEQ ID NO: 327 - protein, 9 aa, synthetic construct
VVVGACGVG

SEQ ID NO: 328 - protein, 9 aa, synthetic construct
VGACGVGKS

SEQ ID NO: 329 - protein, 9 aa, synthetic construct
VVVGACGVG

SEQ ID NO: 330 - protein, 9 aa, synthetic construct
VGADGVGKS

SEQ ID NO: 331 - protein, 9 aa, synthetic construct
VVVGADGVG

SEQ ID NO: 332 - protein, 9 aa, synthetic construct
GAGDVGKSA

SEQ ID NO: 333 - protein, 9 aa, synthetic construct
VVGAGDVGK

SEQ ID NO: 334 - protein, 31 aa, synthetic construct
MTEYKLVVVGACGVGKSALTIQLIQNHFVDE

SEQ ID NO: 335 - protein, 8 aa, synthetic construct
VGACGVGK

SEQ ID NO: 336 - protein, 31 aa, synthetic construct
MTEYKLVVVGADGVGKSALTIQLIQNHFVDE

SEQ ID NO: 337 - protein, 8 aa, synthetic construct
VGADGVGK

SEQ ID NO: 338 - protein, 31 aa, synthetic construct
TEYKLVVVGAGDVGKSALTIQLIQNHFVDEY

SEQ ID NO: 339 - protein, 8 aa, synthetic construct
GAGDVGKS

SEQ ID NO: 340 - protein, 31 aa, synthetic construct
MTEYKLVVVGAGCVGKSALTIQLIQNHFVDE

SEQ ID NO: 341 - protein, 31 aa, synthetic construct; identical to SEQ ID NO: 334

SEQ ID NO: 342 - protein, 31 aa, synthetic construct; identical to SEQ ID NO: 336

SEQ ID NO: 343 - protein, 31 aa, synthetic construct
MTEYKLVVVGAGDVGKSALTIQLIQNHFVDE

SEQ ID NO: 344 - protein, 31 aa, synthetic construct
MTEYKLVVVGAVGVGKSALTIQLIQNHFVDE

SEQ ID NO: 345 - protein, 31 aa, synthetic construct
TCLLDILDTAGHEEYSAMRDQYMRTGEGFLC

SEQ ID NO: 346 - protein, 31 aa, synthetic construct; identical to SEQ ID NO: 334

SEQ ID NO: 347 - protein, 8 aa, synthetic construct
VGACGVGK

SEQ ID NO: 348 - protein, 31 aa, synthetic construct; identical to SEQ ID NO: 336

SEQ ID NO: 349 - protein, 8 aa, synthetic construct
VGADGVGK

SEQ ID NO: 350 - protein, 31 aa, synthetic construct; identical to SEQ ID NO: 338

SEQ ID NO: 351 - protein, 8 aa, synthetic construct
GAGDVGKS

SEQ ID NO: 352 - protein, 177 aa, synthetic construct
CINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNR

SEQ ID NO: 353 - protein, 31 aa, synthetic construct; identical to SEQ ID NO: 340

SEQ ID NO: 354 - protein, 31 aa, synthetic construct; identical to SEQ ID NO: 334

SEQ ID NO: 355 - protein, 31 aa, synthetic construct; identical to SEQ ID NO: 336

SEQ ID NO: 356 - protein, 31 aa, synthetic construct; identical to SEQ ID NO: 343

SEQ ID NO: 357 - protein, 31 aa, synthetic construct; identical to SEQ ID NO: 344

SEQ ID NO: 358 - protein, 31 aa, synthetic construct; identical to SEQ ID NO: 345

SEQ ID NO: 359 - protein, 31 aa, synthetic construct; identical to SEQ ID NO: 340

SEQ ID NO: 360 - protein, 31 aa, synthetic construct; identical to SEQ ID NO: 334

SEQ ID NO: 361 - protein, 31 aa, synthetic construct; identical to SEQ ID NO: 336

SEQ ID NO: 362 - protein, 31 aa, synthetic construct; identical to SEQ ID NO: 343

SEQ ID NO: 363 - protein, 31 aa, synthetic construct; identical to SEQ ID NO: 344

SEQ ID NO: 364 - protein, 31 aa, synthetic construct; identical to SEQ ID NO: 345

SEQ ID NO: 365 - protein, 177 aa, synthetic construct; identical to SEQ ID NO: 352

SEQ ID NO: 366 - protein, 87 aa, synthetic construct
CINLDWDVIRDKTKTKIESLKEHGPIKNKMSESPNKTVSEEKAKQYLEEFHQTALEHPELSELKTVTGTNPVFAGANYAAWAVNVAQ

SEQ ID NO: 367 - protein, 31 aa, synthetic construct; identical to SEQ ID NO: 340

SEQ ID NO: 368 - protein, 31 aa, synthetic construct; identical to SEQ ID NO: 334

SEQ ID NO: 369 - protein, 31 aa, synthetic construct; identical to SEQ ID NO: 336

SEQ ID NO: 370 - protein, 31 aa, synthetic construct; identical to SEQ ID NO: 343

SEQ ID NO: 371 - protein, 31 aa, synthetic construct; identical to SEQ ID NO: 344

SEQ ID NO: 372 - protein, 31 aa, synthetic construct; identical to SEQ ID NO: 345

SEQ ID NO: 373 - protein, 82 aa, synthetic construct
NLEKTTAALSILPGIGSVMGIADGAVHHNTEEIVAQSIALSSLMVAQAIPLVGELVDIGFAAYNFVESIINLFQVVHNSYNR

SEQ ID NO: 374 - protein, 31 aa, synthetic construct; identical to SEQ ID NO: 345

SEQ ID NO: 375 - protein, 17 aa, synthetic construct
YKLVVVGACGVGKSALT

SEQ ID NO: 376 - protein, 17 aa, synthetic construct
YKLVVVGADGVGKSALT

SEQ ID NO: 377 - protein, 17 aa, synthetic construct
YKLVVVGARGVGKSALT

SEQ ID NO: 378 - protein, 17 aa, synthetic construct
YKLVVVGAVGVGKSALT

SEQ ID NO: 379 - protein, 17 aa, synthetic construct
KLVVVGAGCVGKSALTI

SEQ ID NO: 380 - protein, 17 aa, synthetic construct
LDILDTAGHEEYSAMRD

SEQ ID NO: 381 - protein, 17 aa, synthetic construct
KLVVVGAGDVGKSALTI

SEQ ID NO: 382 - protein, 17 aa, synthetic construct
KLVVVGAGRVGKSALTI

SEQ ID NO: 383 - protein, 31 aa, synthetic construct; identical to SEQ ID NO: 334

SEQ ID NO: 384 - protein, 8 aa, synthetic construct
VGACGVGK

SEQ ID NO: 385 - protein, 31 aa, synthetic construct; identical to SEQ ID NO: 336

SEQ ID NO: 386 - protein, 8 aa, synthetic construct
VGADGVGK

SEQ ID NO: 387 - protein, 31 aa, synthetic construct; identical to SEQ ID NO: 344

SEQ ID NO: 388 - protein, 8 aa, synthetic construct
VGAVGVGK

SEQ ID NO: 389 - protein, 31 aa, synthetic construct
MTEYKLVVVGARGVGKSALTIQLIQNHFVDE

SEQ ID NO: 390 - protein, 8 aa, synthetic construct
VGARGVGK

SEQ ID NO: 391 - protein, 31 aa, synthetic construct
TEYKLVVVGAGCVGKSALTIQLIQNHFVDEY

SEQ ID NO: 392 - protein, 8 aa, synthetic construct
GAGCVGKS

SEQ ID NO: 393 - protein, 31 aa, synthetic construct; identical to SEQ ID NO: 338

SEQ ID NO: 394 - protein, 8 aa, synthetic construct
GAGDVGKS

SEQ ID NO: 395 - protein, 8 aa, synthetic construct
TAGHEEYS

SEQ ID NO: 396 - protein, 31 aa, synthetic construct
TEYKLVVVGAGRVGKSALTIQLIQNHFVDEY

SEQ ID NO: 397 - protein, 8 aa, synthetic construct
GAGRVGKS

SEQ ID NO: 398 - protein, 31 aa, synthetic construct; identical to SEQ ID NO: 334

SEQ ID NO: 399 - protein, 8 aa, synthetic construct
VGACGVGK

SEQ ID NO: 400 - protein, 31 aa, synthetic construct; identical to SEQ ID NO: 336

SEQ ID NO: 401 - protein, 8 aa, synthetic construct
VGADGVGK

SEQ ID NO: 402 - protein, 31 aa, synthetic construct; identical to SEQ ID NO: 344

SEQ ID NO: 403 - protein, 8 aa, synthetic construct
VGAVGVGK

SEQ ID NO: 404 - protein, 31 aa, synthetic construct; identical to SEQ ID NO: 389

SEQ ID NO: 405 - protein, 8 aa, synthetic construct
VGARGVGK

SEQ ID NO: 406 - protein, 31 aa, synthetic construct; identical to SEQ ID NO: 391

SEQ ID NO: 407 - protein, 8 aa, synthetic construct
GAGCVGKS

SEQ ID NO: 408 - protein, 31 aa, synthetic construct; identical to SEQ ID NO: 338

SEQ ID NO: 409 - protein, 8 aa, synthetic construct
GAGDVGKS

SEQ ID NO: 410 - protein, 31 aa, synthetic construct; identical to SEQ ID NO: 345

SEQ ID NO: 411 - protein, 8 aa, synthetic construct
TAGHEEYS

SEQ ID NO: 412 - protein, 31 aa, synthetic construct; identical to SEQ ID NO: 396

SEQ ID NO: 413 - protein, 8 aa, synthetic construct
GAGRVGKS

SEQ ID NO: 414 - protein, 31 aa, synthetic construct; identical to SEQ ID NO: 336

SEQ ID NO: 415 - protein, 8 aa, synthetic construct
VGADGVGK

SEQ ID NO: 416 - protein, 31 aa, synthetic construct; identical to SEQ ID NO: 334

SEQ ID NO: 417 - protein, 8 aa, synthetic construct
VGACGVGK

SEQ ID NO: 418 - protein, 31 aa, synthetic construct; identical to SEQ ID NO: 344

SEQ ID NO: 419 - protein, 8 aa, synthetic construct
VGAVGVGK

SEQ ID NO: 420 - protein, 31 aa, synthetic construct; identical to SEQ ID NO: 389

SEQ ID NO: 421 - protein, 8 aa, synthetic construct
VGARGVGK

SEQ ID NO: 422 - protein, 31 aa, synthetic construct; identical to SEQ ID NO: 391

SEQ ID NO: 423 - protein, 8 aa, synthetic construct
GAGCVGKS

SEQ ID NO: 424 - protein, 31 aa, synthetic construct; identical to SEQ ID NO: 344

SEQ ID NO: 425 - protein, 31 aa, synthetic construct; identical to SEQ ID NO: 338

SEQ ID NO: 426 - protein, 8 aa, synthetic construct
GAGDVGKS

SEQ ID NO: 427 - protein, 31 aa, synthetic construct; identical to SEQ ID NO: 345

SEQ ID NO: 428 - protein, 31 aa, synthetic construct; identical to SEQ ID NO: 344

SEQ ID NO: 429 - protein, 8 aa, synthetic construct
TAGHEEYS

SEQ ID NO: 430 - protein, 31 aa, synthetic construct; identical to SEQ ID NO: 396

SEQ ID NO: 431 - protein, 8 aa, synthetic construct
GAGRVGKS

SEQ ID NO: 432 - protein, 17 aa, synthetic construct
YKLVVVGACGVGKSALT

SEQ ID NO: 433 - protein, 17 aa, synthetic construct
YKLVVVGADGVGKSALT

SEQ ID NO: 434 - protein, 17 aa, synthetic construct
YKLVVVGAVGVGKSALT

SEQ ID NO: 435 - protein, 17 aa, synthetic construct
YKLVVVGARGVGKSALT

SEQ ID NO: 436 - protein, 17 aa, synthetic construct
KLVVVGAGCVGKSALTI

SEQ ID NO: 437 - protein, 17 aa, synthetic construct
KLVVVGAGDVGKSALTI

SEQ ID NO: 438 - protein, 17 aa, synthetic construct
LDILDTAGHEEYSAMRD

SEQ ID NO: 439 - protein, 17 aa, synthetic construct
KLVVVGAGRVGKSALTI

SEQ ID NO: 440 - protein, 17 aa, synthetic construct
YKLVVVGACGVGKSALT

SEQ ID NO: 441 - protein, 17 aa, synthetic construct
YKLVVVGADGVGKSALT

SEQ ID NO: 442 - protein, 17 aa, synthetic construct
YKLVVVGAVGVGKSALT

SEQ ID NO: 443 - protein, 17 aa, synthetic construct
YKLVVVGARGVGKSALT

SEQ ID NO: 444 - protein, 17 aa, synthetic construct
KLVVVGAGCVGKSALTI

SEQ ID NO: 445 - protein, 17 aa, synthetic construct
LDILDTAGHEEYSAMRD

SEQ ID NO: 446 - protein, 17 aa, synthetic construct
KLVVVGAGDVGKSALTI

SEQ ID NO: 447 - protein, 17 aa, synthetic construct
KLVVVGAGRVGKSALTI

SEQ ID NO: 448 - protein, 17 aa, synthetic construct
YKLVVVGAVGVGKSALT

SEQ ID NO: 449 - protein, 17 aa, synthetic construct
YKLVVVGARGVGKSALT

SEQ ID NO: 450 - protein, 17 aa, synthetic construct
KLVVVGAGCVGKSALTI

SEQ ID NO: 451 - protein, 17 aa, synthetic construct
LDILDTAGHEEYSAMRD

SEQ ID NO: 452 - protein, 17 aa, synthetic construct
YKLVVVGACGVGKSALT

SEQ ID NO: 453 - protein, 17 aa, synthetic construct
YKLVVVGADGVGKSALT

SEQ ID NO: 454 - protein, 17 aa, synthetic construct
KLVVVGAGDVGKSALTI

SEQ ID NO: 455 - protein, 17 aa, synthetic construct
KLVVVGAGRVGKSALTI

SEQ ID NO: 456 - protein, 31 aa, synthetic construct; identical to SEQ ID NO: 336

SEQ ID NO: 457 - protein, 8 aa, synthetic construct
VGADGVGK

SEQ ID NO: 458 - protein, 31 aa, synthetic construct; identical to SEQ ID NO: 334

SEQ ID NO: 459 - protein, 8 aa, synthetic construct
VGACGVGK

SEQ ID NO: 460 - protein, 31 aa, synthetic construct; identical to SEQ ID NO: 344

SEQ ID NO: 461 - protein, 8 aa, synthetic construct
VGAVGVGK

SEQ ID NO: 462 - protein, 31 aa, synthetic construct; identical to SEQ ID NO: 389

SEQ ID NO: 463 - protein, 8 aa, synthetic construct
VGARGVGK

SEQ ID NO: 464 - protein, 31 aa, synthetic construct; identical to SEQ ID NO: 391

SEQ ID NO: 465 - protein, 8 aa, synthetic construct
GAGCVGKS

SEQ ID NO: 466 - protein, 31 aa, synthetic construct; identical to SEQ ID NO: 338

SEQ ID NO: 467 - protein, 8 aa, synthetic construct
GAGDVGKS

SEQ ID NO: 468 - protein, 31 aa, synthetic construct; identical to SEQ ID NO: 345

SEQ ID NO: 469 - protein, 8 aa, synthetic construct
TAGHEEYS

SEQ ID NO: 470 - protein, 31 aa, synthetic construct; identical to SEQ ID NO: 336

SEQ ID NO: 471 - protein, 8 aa, synthetic construct
VGADGVGK

SEQ ID NO: 472 - protein, 31 aa, synthetic construct; identical to SEQ ID NO: 334

SEQ ID NO: 473 - protein, 8 aa, synthetic construct
VGACGVGK

SEQ ID NO: 474 - protein, 8 aa, synthetic construct
VGAVGVGK

SEQ ID NO: 475 - protein, 31 aa, synthetic construct; identical to SEQ ID NO: 389

SEQ ID NO: 476 - protein, 8 aa, synthetic construct
VGARGVGK

SEQ ID NO: 477 - protein, 31 aa, synthetic construct; identical to SEQ ID NO: 345

SEQ ID NO: 478 - protein, 8 aa, synthetic construct
TAGHEEYS

SEQ ID NO: 479 - protein, 31 aa, synthetic construct; identical to SEQ ID NO: 338

SEQ ID NO: 480 - protein, 8 aa, synthetic construct
GAGDVGKS

SEQ ID NO: 481 - protein, 31 aa, synthetic construct
MTEYKLVVVGAAGVGKSALTIQLIQNHFVDE

SEQ ID NO: 482 - protein, 8 aa, synthetic construct
VGAAGVGK

SEQ ID NO: 483 - protein, 31 aa, synthetic construct
RSYGIPFIETSTKTRQRVEDAFYTLVREIRQ

SEQ ID NO: 484 - protein, 8 aa, synthetic construct
ETSTKTRQ

SEQ ID NO: 485 - protein, 31 aa, synthetic construct; identical to SEQ ID NO: 391

SEQ ID NO: 486 - protein, 8 aa, synthetic construct
GAGCVGKS

SEQ ID NO: 487 - protein, 31 aa, synthetic construct
MTEYKLVVVGASGVGKSALTIQLIQNHFVDE

SEQ ID NO: 488 - protein, 8 aa, synthetic construct
VGASGVGK

SEQ ID NO: 489 - protein, 31 aa, synthetic construct
TCLLDILDTAGREEYSAMRDQYMRTGEGFLC

SEQ ID NO: 490 - protein, 8 aa, synthetic construct
TAGREEYS

SEQ ID NO: 491 - protein, 31 aa, synthetic construct; identical to SEQ ID NO: 334

SEQ ID NO: 492 - protein, 8 aa, synthetic construct
VGACGVGK

SEQ ID NO: 493 - protein, 31 aa, synthetic construct; identical to SEQ ID NO: 344

SEQ ID NO: 494 - protein, 8 aa, synthetic construct
VGAVGVGK

SEQ ID NO: 495 - protein, 31 aa, synthetic construct; identical to SEQ ID NO: 336

SEQ ID NO: 496 - protein, 8 aa, synthetic construct
VGADGVGK

SEQ ID NO: 497 - protein, 31 aa, synthetic construct; identical to SEQ ID NO: 345

SEQ ID NO: 498 - protein, 8 aa, synthetic construct
TAGHEEYS

SEQ ID NO: 499 - protein, 31 aa, synthetic construct; identical to SEQ ID NO: 391

SEQ ID NO: 500 - protein, 8 aa, synthetic construct
GAGCVGKS

SEQ ID NO: 501 - protein, 31 aa, synthetic construct; identical to SEQ ID NO: 338

SEQ ID NO: 502 - protein, 8 aa, synthetic construct
GAGDVGKS

SEQ ID NO: 503 - protein, 31 aa, synthetic construct; identical to SEQ ID NO: 481

SEQ ID NO: 504 - protein, 8 aa, synthetic construct
VGAAGVGK

SEQ ID NO: 505 - protein, 31 aa, synthetic construct; identical to SEQ ID NO: 487

SEQ ID NO: 506 - protein, 8 aa, synthetic construct
VGASGVGK

SEQ ID NO: 507 - protein, 31 aa, synthetic construct; identical to SEQ ID NO: 336

SEQ ID NO: 508 - protein, 8 aa, synthetic construct
VGADGVGK

SEQ ID NO: 509 - protein, 31 aa, synthetic construct; identical to SEQ ID NO: 344

SEQ ID NO: 510 - protein, 8 aa, synthetic construct
VGAVGVGK

SEQ ID NO: 511 - protein, 31 aa, synthetic construct; identical to SEQ ID NO: 389

SEQ ID NO: 512 - protein, 8 aa, synthetic construct
VGARGVGK

SEQ ID NO: 513 - protein, 31 aa, synthetic construct; identical to SEQ ID NO: 345

SEQ ID NO: 514 - protein, 8 aa, synthetic construct
TAGHEEYS

SEQ ID NO: 515 - protein, 31 aa, synthetic construct; identical to SEQ ID NO: 489

SEQ ID NO: 516 - protein, 8 aa, synthetic construct
TAGREEYS

SEQ ID NO: 517 - protein, 31 aa, synthetic construct; identical to SEQ ID NO: 391

SEQ ID NO: 518 - protein, 8 aa, synthetic construct
GAGCVGKS

SEQ ID NO: 519 - protein, 31 aa, synthetic construct; identical to SEQ ID NO: 334

SEQ ID NO: 520 - protein, 8 aa, synthetic construct
VGACGVGK

SEQ ID NO: 521 - protein, 31 aa, synthetic construct; identical to SEQ ID NO: 338

SEQ ID NO: 522 - protein, 8 aa, synthetic construct
GAGDVGKS

SEQ ID NO: 523 - protein, 31 aa, synthetic construct; identical to SEQ ID NO: 336

SEQ ID NO: 524 - protein, 8 aa, synthetic construct
VGADGVGK

SEQ ID NO: 525 - protein, 8 aa, synthetic construct
VGAVGVGK

SEQ ID NO: 526 - protein, 31 aa, synthetic construct; identical to SEQ ID NO: 338

SEQ ID NO: 527 - protein, 8 aa, synthetic construct
GAGDVGKS

SEQ ID NO: 528 - protein, 31 aa, synthetic construct; identical to SEQ ID NO: 483

SEQ ID NO: 529 - protein, 8 aa, synthetic construct
ETSTKTRQ

SEQ ID NO: 530 - protein, 31 aa, synthetic construct; identical to SEQ ID NO: 334

SEQ ID NO: 531 - protein, 8 aa, synthetic construct
VGACGVGK

SEQ ID NO: 532 - protein, 31 aa, synthetic construct; identical to SEQ ID NO: 481

SEQ ID NO: 533 - protein, 8 aa, synthetic construct
VGAAGVGK

SEQ ID NO: 534 - protein, 31 aa, synthetic construct; identical to SEQ ID NO: 487

SEQ ID NO: 535 - protein, 8 aa, synthetic construct
VGASGVGK

SEQ ID NO: 536 - protein, 31 aa, synthetic construct; identical to SEQ ID NO: 391

SEQ ID NO: 537 - protein, 8 aa, synthetic construct
GAGCVGKS

SEQ ID NO: 538 - protein, 31 aa, synthetic construct
MTEYKLVMVGADGVGKSALTIQLIQNHFVDE

SEQ ID NO: 539 - protein, 8 aa, synthetic construct
VGADGVGK

SEQ ID NO: 540 - protein, 31 aa, synthetic construct; identical to SEQ ID NO: 334

SEQ ID NO: 541 - protein, 8 aa, synthetic construct
VGACGVGK

SEQ ID NO: 542 - protein, 31 aa, synthetic construct; identical to SEQ ID NO: 344

SEQ ID NO: 543 - protein, 8 aa, synthetic construct
VGAVGVGK

SEQ ID NO: 544 - protein, 31 aa, synthetic construct; identical to SEQ ID NO: 389

SEQ ID NO: 545 - protein, 8 aa, synthetic construct
VGARGVGK

SEQ ID NO: 546 - protein, 31 aa, synthetic construct; identical to SEQ ID NO: 391

SEQ ID NO: 547 - protein, 8 aa, synthetic construct
GAGCVGKS

SEQ ID NO: 548 - protein, 31 aa, synthetic construct; identical to SEQ ID NO: 338

SEQ ID NO: 549 - protein, 8 aa, synthetic construct
GAGDVGKS

SEQ ID NO: 550 - protein, 31 aa, synthetic construct; identical to SEQ ID NO: 345

SEQ ID NO: 551 - protein, 8 aa, synthetic construct
TAGHEEYS

SEQ ID NO: 552 - protein, 5 aa, synthetic construct
GGGGS

SEQ ID NO: 553 - protein, 6 aa, synthetic construct
LLSVGG

SEQ ID NO: 554 - RNA, 61 nt, synthetic construct
  source 1..61: mol_type = other RNA
agtcctccccatcctctccctctgtccctctgtccctctgaccctgcactgtcccagcacc

SEQ ID NO: 555 - RNA, 170 nt, synthetic construct
  source 1..170: mol_type = other RNA
ctcgaggtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattattttaactatgctagc

SEQ ID NO: 556 - RNA, 101 nt, synthetic construct
  source 1..101: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 557 - protein, 189 aa, Homo sapiens [sequence omitted]

(19) United States
(12) Patent Application Publication
(10) Pub. No.: US 2026/0207724 A1
(43) Pub. Date: Jul. 23, 2026

(54) ANTIGENIC PEPTIDE HAVING MULTIPLE KRAS VARIANT PEPTIDES LINKED THERETO, NUCLEIC ACID ENCODING THE SAME, AND USE THEREOF

(71) Applicant: HANMI PHARM. CO., LTD., Hwaseong-si, Gyeonggi-do (KR)

(72) Inventors: Yu Yon KIM, Hwaseong (KR); Inn Ah KIM, Hwaseong (KR); Joo Young DONG, Hwaseong (KR); Seung Hyun SHIN, Hwaseong (KR); Ji Hee LEE, Hwaseong (KR); Chang Gyu LIM, Hwaseong (KR); Seong Ju JEONG, Hwaseong (KR); Young Jin HAN, Hwaseong (KR); Yong Ho HEO, Hwaseong (KR)

(21) Appl. No.: 19/140,699

(22) PCT Filed: Dec. 22, 2023
(86) PCT No.: PCT/KR2023/021488

§ 371 Date: Jun. 18, 2025

(30) Foreign Application Priority Data
Dec. 23, 2022 (KR) 10-2022-0183225
Apr. 12, 2023 (KR) 10-2023-0048320

(51) Int. Cl. A61K 39/00; A61K 38/00; A61P 35/00; C12N 9/14; C12N 15/85

(52) U.S. Cl. CPC A61K 39/001164; A61P 35/00; C12N 9/14; C12N 15/85; A61K 38/00; A61K 2039/53; A61K 2039/55555; C07K 2319/00; C12Y 306/05002

(57) ABSTRACT

Provided are an antigenic peptide linked to multiple KRAS variant peptides, an mRNA encoding the same, a DNA encoding the same, an immunogenic composition including an antigenic peptide linked to multiple KRAS variant peptides or an mRNA encoding the same, and a method of inducing an immune response to KRAS variant peptides in a subject.

### TECHNICAL FIELD

The disclosure relates to an antigenic peptide having multiple KRAS variant peptides linked thereto, a nucleic acid encoding the same, and uses thereof.

### BACKGROUND ART

Nucleic acid vaccines rely on antigens encoded by DNA or RNA. DNA vaccines generally include an antigen-encoding gene inserted into a bacterial plasmid under the control of a eukaryotic promoter. RNA vaccines, on the other hand, utilize messenger RNA (mRNA) or other antigen-encoding RNAs. Similar to protein vaccines, nucleic acid vaccines may be delivered via a variety of routes, such as intramuscular, subcutaneous, mucosal, or transdermal delivery.

DNA vaccines are known to induce weaker immune responses than peptide vaccines, cellular vaccines, viral vector vaccines, and RNA vaccines. In addition, DNA vaccines not only have low immunogenicity, but also have the risk of being inserted into the host genome and inducing oncogenesis. Accordingly, there is a demand for RNA-based vaccines.

### DISCLOSURE

### Technical Problem

An aspect provides an antigenic peptide in which two or more KRAS variant peptides selected from a group consisting of KRAS variant peptides are linked, wherein the antigenic peptide includes G12C, G12D, and G13D variant peptides.

Another aspect provides an mRNA encoding the antigenic peptide.

Another aspect provides a DNA encoding the antigenic peptide.

Another aspect provides an immunogenic composition including the antigenic peptide, or the mRNA encoding the same.

Another aspect provides a method of inducing an immune response to KRAS variant peptides in a subject, comprising administering to the subject the antigenic peptide, or the mRNA encoding the same.

### Technical Solution

As used herein, with respect to a polypeptide or polynucleotide, the term “identity” refers to a relationship between the sequences of two or more polypeptides or polynucleotides as determined by comparing the sequences. The identity represents the degree of sequence relatedness, which is determined by the number of matches between strings of two or more amino acid residues or nucleotide residues. The identity of related polypeptides or polynucleotides may be calculated by known methods. The term “% identity” as applied to a polypeptide or polynucleotide is defined as the percentage of residues in the candidate amino acid sequence or nucleotide sequence that are identical with residues of a second sequence after aligning the candidate amino acid or nucleotide sequences with the second sequence, introducing gaps if necessary, to obtain the highest percent identity. Methods and programs for such alignments are known. Examples of such programs include BLAST, the Smith-Waterman algorithm, or the Needleman-Wunsch algorithm.

As used herein, the term “5′-untranslated region (5′-UTR)” refers to a region of an mRNA that is directly upstream (i.e., 5′) from the initiation codon of the mRNA transcript, which is the first codon translated by the ribosome, and does not encode a polypeptide.

As used herein, the term “3′-untranslated region (3′-UTR)” refers to a region of an mRNA that is directly downstream (i.e., 3′) from a stop codon, which signals translation termination in the mRNA transcript, and does not encode a polypeptide.

As used herein, the term “open reading frame (ORF)” refers to a contiguous region of nucleic acid that begins with an initiation codon, for example, a methionine codon (ATG), and ends with a stop codon, for example, TAA, TAG or TGA, and encodes a polypeptide.

As used herein, the term “polyadenylate sequence”, or “polyA tail”, refers to a region of an mRNA located downstream, for example directly downstream (i.e., 3′) from a 3′ UTR, that contains multiple consecutive adenosine monophosphates. The polyA tail may include 10 to 300 adenosine monophosphate units. For example, the polyA tails may include 10, 20, 30, 40, 50, 60, 70, 80, 90, 100, 110, 120, 130, 140, 150, 160, 170, 190, 200, 210, 220, 230, 240, 250, 260, 270, 280, 290, or 300 adenosine monophosphate units. The polyA tail may include, for example, 50 to 250 adenosine monophosphate units. In vivo, such as in a cell or an organism, the polyA tail may serve to protect the mRNA from enzymatic degradation, for example, by enzymes present in the cytoplasm, and may assist in transcription termination, export of the mRNA from the nucleus, and translation.

As used herein, the term “operatively linked” refers to nucleotide sequences being joined on a single nucleic acid fragment such that one function is affected by the other. For example, a promoter is operably linked to a coding sequence (e.g., an ORF) when it may affect the expression of the coding sequence (i.e., the coding sequence is under the transcriptional control of the promoter). The coding sequence may be operably linked to a regulatory sequence in either the sense or antisense direction.

As used herein, unless otherwise stated with respect to the position of the nucleotide sequence, the sequence are connected or positioned in the direction from 5′ to 3′ ends.

As used herein, the term “vector” or “nucleic acid construct” refers to any nucleic acid capable of transporting a gene, ORF, or DNA fragment into a cell. The vector may, for example, be capable of replication within a cell. The vector may be an artificial chromosome such as a virus, bacteriophage, provirus, plasmid, phagemid, transposon, yeast artificial chromosome (YAC), bacterial artificial chromosome (BAC), or plant artificial chromosome (PLAC).

A first aspect provides an antigenic peptide in which two or more KRAS variant peptides selected from a group consisting of KRAS variant peptides are linked, wherein the antigenic peptide includes G12C, G12D, and G13D variant peptides.

The KRAS variant peptide may further include G12A, G12V, G12R, G12S, G13C, G13D, G13R, Q61H, Q61R, A146T, or a combination thereof. The KRAS variant peptide may have 7 to 50 amino acid residues. The variant peptide may include, relative to the wild-type sequence, a mutation residue, and also include an extension sequence 1 extending from the mutation residue toward the N-terminus by 3 to 23 amino acids and an extension sequence 2 extending from the mutation residue toward the C-terminus by 3 to 23 amino acids. The variant peptides may be, for example, Antigens 1 to 22 described in Table 1. The extension sequence 1 may be 3 to 12 amino acids in length, and the extension sequence 2 may be 3 to 23 amino acids in length, for example, 3 to 19 amino acids in length. The extension sequence 1 may be 3, 5, 7, 8, 9, 11 or 12 amino acids in length, and the extension sequence 2 may be 3, 4, 5, 8, 9, 11, 18 or 19 amino acids in length.

The antigen peptide may include a plurality of the variant peptides linked through peptide bonds. For example, the antigen peptide may be 3, 4, 5, 6, 7, 8, 9, 10, 11, 12, 13, 14, 15, 16, 17, 18, 19, 20, 21, or 22 of the variant peptides linked via peptide bonds. The linkage may be such that the variant peptides are directly connected or connected via a peptide linker. The peptide linker may be a G4S (SEQ ID NO: 552) or LLSVGG (SEQ ID NO: 553) linker. Each variant peptide included in the antigenic peptide may include mutation residues at the same position or at different positions. For example, the antigen peptide may include mutation residues at 1, 2, 3, or 4 mutation positions. The variation position may be C12, C13, Q61 or A146.

In an embodiment, the variant peptide may be 8, 17, or 31 amino acids (aa) in length. The antigen peptide may further include one or more of the G12V, G12R, G13C, G13R, and Q61H variant peptides.

In an embodiment, the antigen peptide may include G12C, G12D, G12V, G12R, G13C, G13D, and Q61H variant peptides, each of which may be 17 aa in length.

In an embodiment, the antigen peptide may include only variant peptides that are 8 aa or 31 aa in length. The antigen peptide may include linked variant peptides having the same mutation residue, with lengths of 8 aa and 31 aa. The antigen peptides may Include G12C, G12D, G12V, G12R, G13C, G13D, G13R and Q61H variant peptides.

The antigen peptide may have a signal peptide linked to the N-terminus. The signal peptide may be a secretory signal peptide. The signal peptide may include an IgK signal peptide, an AZU1 signal peptide, a LAMP1 signal peptide, or an MHC-I signal peptide.

The antigenic peptide may be linked to an AMP1 transmembrane domain, an MHC-I trafficking domain, a diphtheria toxin (DTT), a diphtheria toxin (DTT) domain (1-87 aa), or a diphtheria toxin (DTT) domain (96-177 aa). The antigenic peptide may be linked to an AMP1 transmembrane domain, diphtheria toxin (DTT), or a diphtheria toxin (DTT) domain (1-87 aa) at the N-terminus, and may be linked to an MHC-I trafficking domain, or a diphtheria toxin (DTT) domain (96-177 aa) at the N-terminus.

The antigenic peptide may be any one of the amino acid sequences of SEQ ID NOs: 23 to 44.

A second aspect provides an mRNA encoding the antigenic peptide.

The mRNA may include an expression regulatory sequence or a sequence that influences stabilization. The expression regulatory sequence or stabilization-influencing sequences may be 5′-UTR, 3′-UTR, and/or polyA. The 5′-UTR, 3′-UTR, and polyA may be natural or non-natural sequences. These sequences may be operably linked to ORFs or to each other.

The 5′-UTR may have a sequence identity of at least 70%, for example, at least 80%, at least 90%, or at least 95%, with the nucleotide sequence of SEQ ID NO: 553. The 5′-UTR may include SEQ ID NO: 553.

The 5′UTR may be derived from an mRNA encoding a protein selected from the group consisting of p53, OX40L, albumin, serum amyloid A, Apolipoprotein A/B/E, transferrin, alpha fetoprotein, erythropoietin, Factor VIII, MyoD, Myosin, Myoglobin, Myogenin, Herculin, Tie-1, CD36, C/EBP, AML1, G-CSF, GM-CSF, CD1 lb, MSR, Fr-1, i-NOS, CD45, CD18, CD36, GLUT4, ACRP30, adiponectin, PA/B/C/D, ORM1, HPX, FGA, CYP2E1, C3, ASC, APOA2, ALB, AGXT, α-globin, β-globin, tyrosine hydroxylase, and collagen.

The 3′-UTR may have a sequence identity of at least 70%, for example, at least 80%, at least 90%, or at least 95%, with the nucleotide sequence of SEQ ID NO: 554. The 3′-UTR may be derived from an mRNA encoding a protein selected from the group consisting of p53, OX40L, albumin, serum amyloid A, Apolipoprotein A/B/E, transferrin, alpha fetoprotein, erythropoietin, Factor VIII, MyoD, Myosin, Myoglobin, Myogenin, Herculin, Tie-1, CD36, C/EBP, AML1, G-CSF, GM-CSF, CD1 lb, MSR, Fr-1, i-NOS, CD45, CD18, CD36, GLUT4, ACRP30, adiponectin, PA/B/C/D, ORM1, HPX, FGA, CYP2E1, C3, ASC, APOA2, ALB, AGXT, α-globin, β-globin, tyrosine hydroxylase, and collagen.

The mRNA may include one or more modified nucleotides. The modified polynucleotide, when introduced into a cell or organism, may exhibit reduced degradation in the cell or organism compared to an unmodified one. Additionally, the modified polynucleotide, when introduced into a cell or organism, may exhibit reduced immunogenicity (e.g., reduced innate response) in the cell or organism compared to the unmodified one.

A nucleotides having a modified cytosine may include N4-acetyl-cytidine (ac4C), 5-methyl-cytidine (m5C), 5-halo-cytidine (eg, 5-iodo-cytidine), 5-hydroxymethyl-cytidine (hm5C), 1-methyl-pseudoisocytidine, 2-thio-cytidine (s2C), 2-thio-5-methyl-cytidine, or a combination thereof.

A nucleotide having a modified adenine may include 7-deaza-adenine, 1-methyl-adenosine (m1A), 2-methyl-adenine (m2A), N6-methyl-adenosine (m6A), or a combination thereof.

A nucleotide having a modified guanine may include inosine (I), 1-methyl-inosine (m11), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, or a combination thereof.

A nucleotides having the chemical modification may be pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, 2′-O-methyluridine, or a combination thereof. The chemical modification may be located at the 5′ position of uracil. The chemical modification may be a modification of uracil to N1-methylpseudouridine. The chemical modification may be a modification of uracil to N1-ethylpseudouridine.

The mRNA may include a 5′ cap. The term “5′-cap” refers to a cap structure found at the 5′-end of an mRNA molecule. The 5′-cap may include a guanosine nucleotide linked to the mRNA via an unusual 5′ to 5′ triphosphate linkage. The guanosine may be methylated at the 7-position, for example, m7G, or 3′-O-Me-m7G. The term “conventional 5′ cap” refers to naturally occurring RNA 5′ caps, such as 7-methylguanosine (m7G). As used herein, the term “5′ cap” includes 5′ cap analogs that resemble an RNA cap structure, and when attached to RNA, e.g., in vivo and/or in cells, are modified to have the ability to stabilize RNA. The provision of a 5′ cap or 5′ cap analog to RNA may be performed by in vitro transcription of a DNA template in the presence of the 5′ cap or 5′ cap analog, thereby co-transcriptionally inserting the 5′ cap or 5′ cap analog into the RNA strand from which it is produced, or by post-transcriptionally using a capping enzyme, such as the vaccinia virus capping enzyme. The 5′-terminal cap structure may be, for example, 7 mG (5′) ppp (5′) ImpNp, 3′-O-Me-m7G (5′) ppp (5′) G, m7 (3′OMeG) (5′) ppp (5′) (2′OMeA) or m7G (5′) ppp (5′) (2′OMeA) pG.

The mRNA may have one or more uridines (U) substituted with N1-methyl-pseudouridine. In the mRNA, the GA at the 5′ end may have a cap structure of m7 (3′OMeG) (5′) ppp (5′) (2′OMeA). In the mRNA, all U's may be substituted with N1-methyl-pseudouridine. The mRNA may have all U's substituted with N1-methyl-pseudouridine, and the GA at the 5′ end may have a cap structure of m7 (3′OMeG) (5′) ppp (5′) (2′OMeA).

The mRNA may be codon-optimized for a host cell. The codon optimization may vary depending on the host cell in which the mRNA is produced.

The mRNA may include any one of the nucleotide sequences of SEQ ID NOs: 1 to 22. Additionally, the mRNA may include any one of the nucleotide sequences of SEQ ID NOs: 1 to 23. Additionally, the mRNA may include any one of the nucleotide sequences of SEQ ID NOs: 296 to 300.

A third aspect provides DNA encoding the antigenic peptide.

The DNA may further include expression regulatory and restriction enzyme sites. The expression reguartory element may be a promoter. The expression regulatory element may be operably linked to the ORF or to another expression regulatory element.

The 3′-UTR coding sequence may include a recognition sequence for a restriction enzyme. The estriction enzyme may be Xhol, Nhel, or Xhol and Nhel. The promoter may be operably linked to a sequence encoding the 5′-UTR. The 5-UTR, the antigen peptide coding sequence, and the 3′-UTR may be linked to form a common transcript when transcribed in vitro or in vivo. The sequence encoding the 3′-UTR may be operably linked to a polyA sequence.

The sequence encoding the 5′-UTR may be a sequence encoding the sequence of SEQ ID NO: 553, and the sequence encoding the 3′-UTR may include a sequence encoding the sequence of SEQ ID NO: 554. In this case, the sequence encoding the KRAS antigen peptide may have any one of the nucleotide sequences of SEQ ID NOs: 1 to 22 (Including only the sequence of the coding region). Additionally, the polyA sequence may include the sequence of SEQ ID NO: 556.

As used herein, the term “nucleic acids capable of being transcribed to produce a common transcript” refers to nucleic acid sequences that are functionally or operatively linked to each other such that when transcribed, an RNA molecule is formed that includes a transcript in which the nucleic acid sequences are covalently linked to each other. The transcription may be performed, where appropriate, after linearization of the nucleic acid molecule, for example, by restriction enzyme digestion of a closed circular nucleic acid molecule, under the control of a promoter. The common transcript may, where appropriate, be separated by sequences located between the nucleic acid sequences.

The DNA molecule may be a closed circular molecule or a linear molecule.

The DNA molecule may further include one or more selected from the group consisting of (I) a reporter gene, (Ii) a selectable marker, and (iii) an origion of replication. The DNA molecule may be a vector. The vector may be a cloning vector or an expression vector. The DNA molecule may be suitable for in vitro transcription of mRNA after linearization.

A fourth aspect provides an immunogenic composition including the antigen peptide or the mRNA encoding the antigen peptide as an active ingredient.

The antigen peptide and the mRNA encoding it are as described in the first aspect and the second aspect.

The composition may be for inducing an immune response against KRAS variant peptides. The composition may be for preventing or treating cancer. The subject to be prevented or treated may have a KRAS mutation. The mutation may have a G12C, G12D, G12V, G12R, G13C, G13D, G12R, Q61H, Q61R, A147R, or a combination mutation thereof.

The composition may be a pharmaceutical composition including a pharmaceutically acceptable carrier, diluent or excipient.

The composition may be for treating cancer. The cancer may be a cancer containing a KRAS mutation. The cancer may be a hematologic cancer or a solid tumor.

The hematologic cancer may be leukemia, malignant lymphoma, multiple myeloma, or aplastic anemia. The solid tumor may be a brain tumor, benign astrocytoma, malignant astrocytoma, pituitary adenoma, meningioma, brain lymphoma, oligodendroglioma, intracranial tumor, ependymoma, brainstem tumor, head and neck tumor, laryngeal cancer, oropharyngeal cancer, nasal cancer, nasopharyngeal cancer, salivary gland cancer, hypopharyngeal cancer, thyroid cancer, oral cancer, thoracic tumor, lung cancer, thymic cancer, mediastinal tumor, esophageal cancer, breast cancer, male breast cancer, abdominal tumor, stomach cancer, liver cancer, gallbladder cancer, biliary tract cancer, pancreatic cancer, small intestine cancer, colon cancer, anal cancer, bladder cancer, kidney cancer, male genital tumor, penile cancer, prostate cancer, female genital tumor, cervical cancer, endometrial cancer, ovarian cancer, uterine sarcoma, vaginal cancer, female external genital cancer, female urethral cancer, or skin cancer. The lung cancer may be small cell lung cancer or non-small cell lung cancer.

The composition may be administered to a subject and the mRNA may be translated in vivo to produce the antigenic peptide.

The composition may be contacted with a cell, tissue or subject in an “effective amount” to induce an immune response.

The effective amount may be determined based on the target tissue, target cells, means of administration, properties of the mRNA, such as size and content of modified nucleosides, and other components of the composition.

The composition may be administered together with other prophylactic or therapeutic compounds. The prophylactic or therapeutic compound may be, for example, an anticancer agent. As used herein, the anticancer agent may be any known agent.

The composition may have a systemic or topical administration formulation. The topical administration may be administered locally to the tissue where the cancer exists. The tissue where the cancer is present may be intramuscular, subcutaneous, intradermal, nasal, or pulmonary.

The composition may include one or more pharmaceutically acceptable carriers, diluents or excipients. The mRNA in the composition may be formulated or complexed with the excipient. The carrier, diluent or excipient may be known in the art.

The relative amounts of the active ingredient, pharmaceutically acceptable excipients, and/or other additional ingredients included in the composition may vary depending on the nature, size, and/or condition of the subject to be treated and the route of administration. For example, the composition can comprise from 0.1% to 100%, for example, from 0.5% to 50%, from 1.0% to 30%, from 5.0% to 80%, or greater than 80% (w/w) of the active ingredient.

The composition may be formulated as nanoparticles. The nanoparticles may be known to be capable of delivering mRNA into cells. For example, the nanoparticle may be a lipid nanoparticle (LNP). The composition may be formulated as a lipid nanoparticle (LNP) or bound to an LNP. The binding may include binding within or on the surface of the LNP. For example, the composition may be formulated within a lipid polycation complex or bound to a lipid polycation complex. The lipid polycation complex is also referred to as a cationic lipid nanoparticle. The polycation may include a cationic polypeptide, such as MC3, Lipid 319, C12-200, 5A2-SC8, 3060i10, Moderna Lipid 5, Acuitas A9, SM-102, ALC-0315, Arcturus Lipid 2,2 (8,8) 4C CH3, Genevant CL1, polylysine, polyornithine, and/or polyarginine. Additionally, the composition may be formulated within or bound to a lipid nanoparticles including non-cationic lipids such as distearoylphosphatidylcholine (DSPC), 1,2-distearoyl-sn-glycero-3-phosphocholine (DSPC), dipalmitoylphosphatidylcholine (DPPC), sterols such as cholesterol, or dioleoylphosphatidylethanolamine (DOPE). Additionally, the composition may be formulated with or bound to lipid nanoparticles comprising PEG-lipids such as 1,2-dimyristoyl-rac-glycero-3-methoxypolyethylene glycol-2000 (PEG2000-DMG), 2-[(polyethylene glycol)-2000]-N,N-ditetradecylacetamide (ALC-0159), polyethylene glycol dimethacrylate (PEG-DMA).

The lipid nanoparticle formulations may include a cationic lipid, a phospholipid, a sterol such as cholesterol, a PEG-lipid, or a combination thereof. The lipid nanoparticle formulations may include, for example, a cationic lipid, a phospholipid, a sterol such as cholesterol, and a PEG-lipid. Additionally, the lipid nanoparticles may include a PEG-modified lipid, a non-cationic lipid, a sterol, and an ionizable lipid, or a combination thereof. The lipid nanoparticle may include 0.5 mol % to 15 mol % PEG-modified lipid, 5 mol % to 25 mol % non-cationic lipid, 25 mol % to 55 mol % sterol, and 20 mol % to 60 mol % ionizable lipid. The PEG-modified lipid may be 1,2 dimyristoyl-sn-glycerol methoxypolyethylene glycol (PEG2000-DMG), the non-cationic lipid may be 1,2 distearoyl-sn-glycerol-3-phosphocholine (DSPC), the sterol may be cholesterol, and the ionizable cationic lipid may be Compound 1 having the following structure.

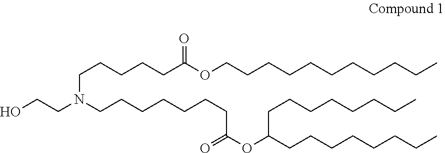 [SMILES: CCCCCCCCCCCOC(=O)CCCCCN(CCO)CCCCCCCC(=O)OC(CCCCCCCC)CCCCCCCC]

The PEG-modified lipid may be Compound 2 (ALC-0159) having the following structure, the non-cationic lipid may be 1,2 distearoyl-sn-glycerol-3-phosphocholine (DSPC), the sterol may be cholesterol, and the ionizable cationic lipid may be Compound 3 (ALC-0315) having the following structure.

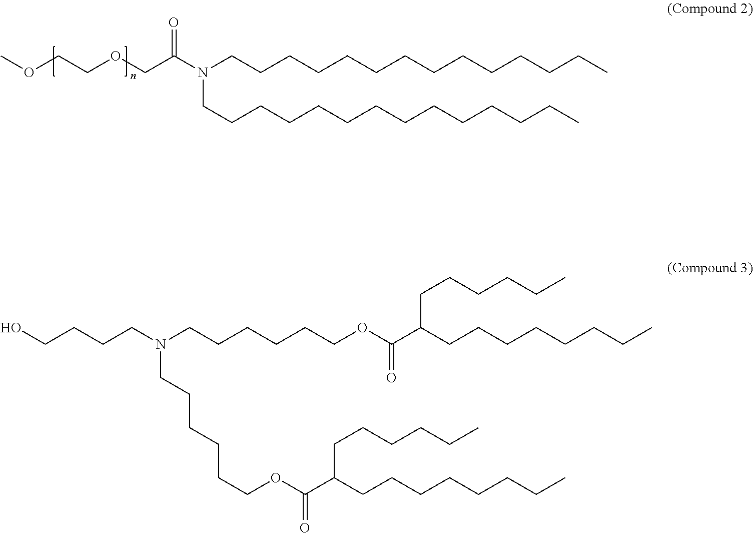

A fifth aspect provides a method of inducing an immune response in a subject including administering the antigenic peptide or the mRNA to the subject. The method may be for preventing or treating cancer.

The administration may be administered in an amount effective to induce an immune response in the subject. The administration may be administered in an amount effective to prevent or treat cancer in the subject. The administration may be systemic or topical. The topical administration may be administered locally to the tissue where the cancer is present. The tissue where the cancer is present may be intramuscular, subcutaneous, intradermal, nasal, or pulmonary. The administration may be parenteral or oral.

The subject may be a mammal, including a human. The administration may be local administration of the mRNA molecule either intracellularly or extracellularly to a tissue where cells with high levels of KRAS mutations are present.

The method may be to produce the antigen peptide in cells of an individual to inhibit cancer cell proliferation. Additionally, the method may be for treating a disease in the subject. The antigenic peptide may induce an immune response against cancer in the subject.

### Advantageous Effects

The antigenic peptides in which two or more KRAS variant peptides are linked according to an aspect may be used to efficiently induce an immune response in a subject.

The mRNA encoding the antigenic peptide according to an aspect may be used to efficiently induce an immune response in a subject.

The DNA encoding the antigenic peptide according to an aspect may be used to produce the antigenic peptide.

The immunogenic composition according to an aspect may be used to induce an immune response in a subject.

According to a method of inducing an immune response to a KRAS variant peptide in a subject according to an aspect, an immune response to a KRAS variant peptide may be efficiently induced in the subject.

### DESCRIPTION OF DRAWINGS

FIG. 1 is a diagram showing a vector obtained through the same process as in Examples 1 and 2.

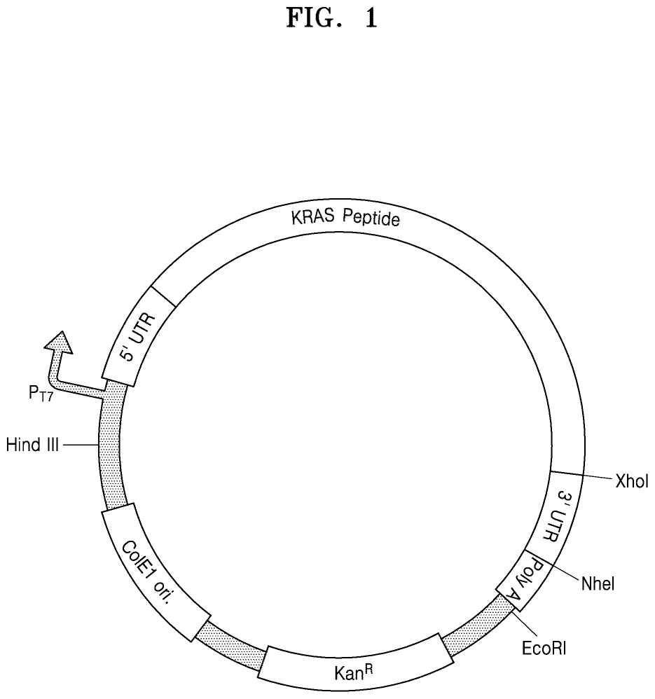

FIG. 2 is a diagram showing tumor size and body weight changes after administration of an anticancer vaccine containing KRAS mRNA encoding Antigen 2 in a Lewis lung cancer mouse model.

FIG. 3 is a diagram showing changes in immune cell distribution within tumor tissue cells after administration of an anticancer vaccine containing KRAS mRNA encoding Antigen 2 in a Lewis lung cancer mouse model.

FIG. 4 is a diagram showing tumor size and body weight changes after administration of an anticancer vaccine containing KRAS mRNA encoding Antigen 6 in a Lewis lung cancer mouse model.

FIG. 5 is a diagram showing changes in CD8 T cell frequency among immune cells in tumor tissue after administration of an anticancer vaccine containing KRAS mRNA encoding Antigen 6 in a Lewis lung cancer mouse model.

FIG. 6 is a diagram showing tumor size and body weight changes after administration of an anticancer vaccine containing KRAS mRNA encoding Antigen 14 or 15 in a Lewis lung cancer mouse model.

FIG. 7A is a photograph showing the results of co-culturing PBMCs expressing KRAS variant peptide-linked Antigens 2 and 6 with target cells.

FIG. 7B is a diagram showing the results of measuring target cell viability when PBMCs expressing KRAS variant peptide-linked Antigens 2 and 6 are co-cultured with target cells.

FIG. 8A is a photograph showing the results of co-culturing PBMCs expressing KRAS variant peptide-linked Antigens 15 and 17 with target cells.

FIG. 8B is a diagram showing the results of measuring the viability of target cells when PBMCs expressing KRAS variant peptide-linked Antigens 15 and 17 are co-cultured with the target cells.

FIG. 9 is a diagram showing the results of a Western blot using an anti-RAS (G12D mutant) monoclonal antibody against proteins expressed in LL/2 cells, which are a Lewis lung carcinoma cell line, into which mRNAs encoding KRAS variant peptide-linked Antigens 6, 14, and 15 were introduced.

### BEST MODE

Hereinafter, the present disclosure will be described in more detail through examples. However, these examples are intended to exemplify the present disclosure and the scope of the present disclosure is not limited to these examples.

### Example 1: Anticancer Agent Sequences Designed to Enhance T Cell Activation Against KRAS Mutant Sequences

An antigen including multiple KRAS variant peptides was designed for the treatment of cancer patients with KRAS mutations. The variant peptides include a mutation residue based on the KRAS wild-type sequence (SEQ ID NO: 557) and includes an extension sequence 1 extending from the mutation residue toward the N-terminus, and an extension sequence 2 extending from the mutation residue toward the C-terminus. The mutation residues include G12A, G12C, G12D, G12V, G12R, G12S, G13C, G13D, G13R, Q61H, Q61R or A146T. Extended sequence 1 may have a length of 3 to 23 amino acids. Extended sequence 2 may have a length of 3 to 23 amino acids.

The variant peptide may be represented as E1-M-E2, where M represents the mutation residue, and E1 and E2 represent an extension sequence 1 extending from the mutation residue to the N-terminus and an extension sequence 2 extending from the mutation residue to the C-terminus, respectively. In the variant peptide E1-M-E2, M may be G12A, G12C, G12D, G12V, G12R, G12S, G13C, G13D, Q61H, Q61R or A146T, E1 may be 3 to 12 amino acids in length, and E2 may be 3 to 23, for example, 3 to 19 amino acids in length. E1 may have a length of 3, 5, 7, 8, 9, 11, or 12 amino acids, and E2 may have a length of 3, 4, 5, 8, 9, 11, 18, or 19 amino acids.

The antigen peptide may include a plurality of the variant peptides linked through peptide bonds. For example, the antigen peptide may include 3, 4, 5, 6, 7, 8, 9, 10, 11, 12, 13, 14, 15, 16, 17, 18, 19, 20, 21, or 22 variant peptides linked via peptide bonds. The linkage may be direct between the variant peptides or via peptide linkers. The peptide linker may be a G4S linker (SEQ ID NO: 552) or an LLSVGG linker (SEQ ID NO: 553). Each variant peptide included in the antigen peptide may include mutation residues at the same position or at different positions. For example, the antigenic peptide may include mutation residues at 1, 2, 3, or 4 mutation positions. The mutation position may be C12, C13, Q61 or A146.

Additionally, the antigenic peptides may all contain either the same mutation residue at the same mutation position, or different mutation residues at the same mutation position.

The antigenic peptide may be linked to a secretory signal peptide. The secretory signal peptide may be removed during the secretion process. The antigenic peptide may be linked to a secretory signal peptide at its N-terminus. The signal peptide may be an IgK signal peptide, an AZU1 signal peptide, a LAMP1 signal peptide, or an MHC-I signal peptide. Additionally, the antigenic peptide may be linked to a LAMP1 transmembrane domain, an MHC-I trafficking domain, a diphtheria toxin (DTT) domain (1-87aa), or a diphtheria toxin (DTT) domain (96-177aa). The antigenic peptide may be linked at is N-terminus to a LAMP1 transmembrane domain, an MHC-I trafficking domain, or a diphtheria toxin (DTT) domain (1-87aa). The antigenic peptide may be linked at its C-terminus to a diphtheria toxin (DTT) domain (96-177aa).

Table 1 shows the KRAS variant peptide composition and corresponding amino acid sequences (SEQ ID NOs: 23 to 44) of Antigens 1 to 22 prepared in this example. In Table 1, SEQ ID NOs: 1 to 22 listed under the column titled “KRAS Variant Peptide Composition” represent the nucleotide sequences of the ORF mRNA for each antigen peptide.

TABLE 1

| Antigen | Peptide name | Amino acid sequence |
|---|---|---|
| 1 | (11-G12R-11)(9-G12D- | MTEYKLVVVGARGVGKSALTIQLEYKLVVVGADG |
| | 9)(9-G12V-9)(9-G12C- | VGKSALTIEYKLVVVGAVGVGKSALTIEYKLVVVG |
| | 9)(9-G13D-9)(9-G13C- | ACGVGKSALTIYKLVVVGAGDVGKSALTIQYKLV |
| | 9)(9-Q61H-9)(SEQ ID | VVGAGCVGKSALTIQLLDILDTAGHEEYSAMRDQ |
| | NO: 1) | (SEQ ID NO: 23) |
| 2 | (8-G12C-8)(8-G12D-8) | YKLVVVGACGVGKSALTYKLVVVGADGVGKSAL |
| | (8-G12V-8)(8-G12R-8) | TYKLVVVGAVGVGKSALTYKLVVVGARGVGKSA |
| | (8-G13C-8)(8-G13D-8) | LTKLVVVGAGCVGKSALTIKLVVVGAGDVGKSAL |
| | (8-Q61H-8)(SEQ ID NO: | TILDILDTAGHEEYSAMRD (SEQ ID NO: 24) |
| | 2) | |
| 3 | -(8-G12C-8)-(8-G12D-8)- | LLSVGGYKLVVVGACGVGKSALTLLSVGGYKLVV |
| | (8-G12V-8)-(8-G12R-8)- | VGADGVGKSALTLLSVGGYKLVVVGAVGVGKSA |
| | (8-G13C-8)-(8-G13D-8)- | LTLLSVGGYKLVVVGARGVGKSALTLLSVGGKLV |
| | (8-Q61H-8)- (SEQ ID | VVGAGCVGKSALTILLSVGGKLVVVGAGDVGKS |
| | NO: 3) | ALTILLSVGGLDILDTAGHEEYSAMRDLLSVGG |
| | | (SEQ ID NO: 25) |
| 4 | (3-G12C-5)-(5-G12C-3)- | VGACGVGKSLLSVGGVVVGACGVGLLSVGGVG |
| | (3-G12C-5)-(5-G12C-3)- | ACGVGKSLLSVGGVVVGACGVGLLSVGGVGAC |
| | (3-G12C-5)-(5-G12C-3) | GVGKSLLSVGGVVVGACGVG (SEQ ID NO: 26) |
| | (SEQ ID NO: 4) | |
| 5 | (3-G12C-5)-(5-G12C-3)- | VGACGVGKSLLSVGGVVVGACGVGLLSVGGVG |
| | (3-G12D-5)-(5-G12D-3)- | ADGVGKSLLSVGGVVVGADGVGLLSVGGGAGD |
| | (3-G13D-5)-(5-G13D-3) | VGKSALLSVGGVVGAGDVGK (SEQ ID NO: 27) |
| | (SEQ ID NO: 5) | |
| 6 | (11-G12C-19)-(3-G12C- | MTEYKLVVVGACGVGKSALTIQLIQNHFVDELLS |
| | 4)-(11-G12D-19)-(3- | VGGVGACGVGKLLSVGGMTEYKLVVVGADGVG |
| | G12D-4)-(11-G13D-19)- | KSALTIQLIQNHFVDELLSVGGVGADGVGKLLSV |
| | (3-G13D-4)(SEQ ID NO: | GGTEYKLVVVGAGDVGKSALTIQLIQNHFVDEYL |
| | 6) | LSVGGGAGDVGKS (SEQ ID NO: 28) |
| 7 | (12-G13C-18)-(11- | MTEYKLVVVGAGCVGKSALTIQLIQNHFVDELLS |
| | G12C-19)-(11-G12D- | VGGMTEYKLVVVGACGVGKSALTIQLIQNHFVDE |
| | 19)-(12-G13D-18)-(11- | LLSVGGMTEYKLVVVGADGVGKSALTIQLIQNHF |
| | G12V-19)-(11-Q61H- | VDELLS |
| | 19)(SEQ ID NO: 7) | VGGMTEYKLVVVGAGDVGKSALTIQLIQNHFVDE |
| | | LLSVGGMTEYKLVVVGAVGVGKSALTIQLIQNHF |
| | | VDELLSVGGTCLLDILDTAGHEEYSAMRDQYMR |
| | | TGEGFLC (SEQ ID NO: 29) |
| 8 | (11-G12C-19)(3-G12C- | MTEYKLVVVGACGVGKSALTIQLIQNHFVDEVGA |
| | 4)(11-G12D-19)(3- | CGVGKMTEYKLVVVGADGVGKSALTIQLIQNHFV |
| | G12D-4)(11-G13D-19) | DEVGADGVGKTEYKLVVVGAGDVGKSALTIQLIQ |
| | (3-G13D-4)(SEQ ID NO: | NHFVDEYGAGDVGKS (SEQ ID NO: 30) |
| | 8) | |
| 9 | DTT (12-G13C-18)-(11- | CINLDWDVIRDKTKTKIESLKEHGPIKNKMSESPN |
| | G12C-19)-(11-G12D- | KTVSEEKAKQYLEEFHQTALEHPELSELKTVTGT |
| | 19)-(12-G13D-18)-(11- | NPVFAGANYAAWAVNVAQVIDSETADNLEKTTA |
| | G12V-19)-(11-Q61H-19) | ALSILPGIGSVMGIADGAVHHNTEEIVAQSIALSSL |
| | (SEQ ID NO: 9) | MVAQAIPLVGELVDIGFAAYNFVESIINLFQVVHN |
| | | SYNRMTEYKLVVVGAGCVGKSALTIQLIQNHFVD |
| | | ELLSVGGMTEYKLVVVGACGVGKSALTIQLIQNH |
| | | FVDELLSVGGMTEYKLVVVGADGVGKSALTIQLI |
| | | QNHFVDELLSVGGMTEYKLVVVGAGDVGKSALTI |
| | | QLIQNHFVDELLSVGGMTEYKLVVVGAVGVGKS |
| | | ALTIQLIQNHFVDELLSVGGTCLLDILDTAGHEEY |
| | | SAMRDQYMRTGEGFLC (SEQ ID NO: 31) |
| 10 | (12-G13C-18)-(11- | MTEYKLVVVGAGCVGKSALTIQLIQNHFVDELLS |
| | G12C-19)-(11-G12D- | VGGMTEYKLVVVGACGVGKSALTIQLIQNHFVDE |
| | 19)-(12-G13D-18)-(11- | LLSVGGMTEYKLVVVGADGVGKSALTIQLIQNHF |
| | G12V-19)-(11-Q61H-19)- | VDELLSVGGMTEYKLVVVGAGDVGKSALTIQLIQ |
| | DTT (SEQ ID NO: 10) | NHFVDELLSVGGMTEYKLVVVGAVGVGKSALTIQ |
| | | LIQNHFVDELLSVGGTCLLDILDTAGHEEYSAMR |
| | | DQYMRTGEGFLCLLSVGGCINLDWDVIRDKTKTK |
| | | IESLKEHGPIKNKMSESPNKTVSEEKAKQYLEEF |
| | | HQTALEHPELSELKTVTGTNPVFAGANYAAWAV |
| | | NVAQVIDSETADNLEKTTAALSILPGIGSVMGIAD |
| | | GAVHHNTEEIVAQSIALSSLMVAQAIPLVGELVDI |
| | | GFAAYNFVESIINLFQVVHNSYNR (SEQ ID NO: |
| | | 32) |
| 11 | DTT (12-G13C-18)-(11- | MTEYKLVVVGAGCVGKSALTIQLIQNHFVDELLS |
| | G12C-19)-(11-G12D- | VGGMTEYKLVVVGACGVGKSALTIQLIQNHFVDE |
| | 19)-(12-G13D-18)-(11- | LLSVGGMTEYKLVVVGADGVGKSALTIQLIQNHF |
| | G12V-19)-(11-Q61H-19) | VDELLSVGGMTEYKLVVVGAGDVGKSALTIQLIQ |
| | DTT (SEQ ID NO: 11) | NHFVDELLSVGGMTEYKLVVVGAVGVGKSALTIQ |
| | | LIQNHFVDELLSVGGTCLLDILDTAGHEEYSAMR |
| | | DQYMRTGEGFLCLLSVGGCINLDWDVIRDKTKTK |
| | | IESLKEHGPIKNKMSESPNKTVSEEKAKQYLEEF |
| | | HQTALEHPELSELKTVTGTNPVFAGANYAAWAV |
| | | NVAQVIDSETADNLEKTTAALSILPGIGSVMGIAD |
| | | GAVHHNTEEIVAQSIALSSLMVAQAIPLVGELVDI |
| | | GFAAYNFVESIINLFQVVHNSYNR (SEQ ID NO: |
| | | 33) |
| 12 | (8-G12C-8)(8-G12D-8) | YKLVVVGACGVGKSALTYKLVVVGADGVGKSAL |
| | (8-G12V-8)(8-G12R-8) | TYKLVVVGAVGVGKSALTYKLVVVGARGVGKSA |
| | (8-G13C-8)(8-G13D-8) | LTKLVVVGAGCVGKSALTIKLVVVGAGDVGKSAL |
| | (8-Q61H-8)(8-G13R-8) | TILDILDTAGHEEYSAMRDKLVVVGAGRVGKSAL |
| | (SEQ ID NO: 12) | TI (SEQ ID NO: 34) |
| 13 | (11-G12C-19)-(3-G12C- | MTEYKLVVVGACGVGKSALTIQLIQNHFVDELLS |
| | 4)-(11-G12D-19)-(3- | VGGVGACGVGKLLSVGGMTEYKLVVVGADGVG |
| | G12D-4)-(11-G12V-19)- | KSALTIQLIQNHFVDELLSVGGVGADGVGKLLSV |
| | (3-G12V-4)-(11-G12R- | GGMTEYKLVVVGAVGVGKSALTIQLIQNHFVDEL |
| | 19)-(3-G12R-4)-(11- | LSVGGVGAVGVGKLLSVGGMTEYKLVVVGARGV |
| | G13C-19)-(3-G13C-4)- | GKSALTIQLIQNHFVDELLSVGGVGARGVGKLLS |
| | (11-G13D-19)-(3-G13D- | VGGTEYKLVVVGAGCVGKSALTIQLIQNHFVDEY |
| | 4)-(11-Q61H-19)-(3- | LLSVGGGAGCVGKSLLSVGGTEYKLVVVGAGDV |
| | Q61H-4)-(11-G13R-19)- | GKSALTIQLIQNHFVDEYLLSVGGGAGDVGKSLL |
| | (3-G13R-4)(SEQ ID NO: | SVGGTCLLDILDTAGHEEYSAMRDQYMRTGEGF |
| | 13) | LCLLSVGGTAGHEEYSLLSVGGTEYKLVVVGAG |
| | | RVGKSALTIQLIQNHFVDEYLLSVGGGAGRVGKS |
| | | (SEQ ID NO: 35) |
| 14 | (11-G12C-19)(3-G12C- | MTEYKLVVVGACGVGKSALTIQLIQNHFVDEVGA |
| | 4)(11-G12D-19)(3- | CGVGKMTEYKLVVVGADGVGKSALTIQLIQNHFV |
| | G12D-4)(11-G12V-19) | DEVGADGVGKMTEYKLVVVGAVGVGKSALTIQLI |
| | (3-G12V-4)(11-G12R- | QNHFVDEVGAVGVGKMTEYKLVVVGARGVGKS |
| | 19)(3-G12R-4)(11- | ALTIQLIQNHFVDEVGARGVGKTEYKLVVVGAGC |
| | G13C-19)(3-G13C-4) | VGKSALTIQLIQNHFVDEYGAGCVGKSTEYKLVV |
| | (11-G13D-19)(3-G13D- | VGAGDVGKSALTIQLIQNHFVDEYGAGDVGKSTC |
| | 4)(11-Q61H-19)(3- | LLDILDTAGHEEYSAMRDQYMRTGEGFLCTAGH |
| | Q61H-4)(11-G13R-19) | EEYSTEYKLVVVGAGRVGKSALTIQLIQNHFVDE |
| | (3-G13R-4)(SEQ ID NO: | YGAGRVGKS (SEQ ID NO: 36) |
| | 14) | |
| 15 | (11-G12D-19)-(3-G12D- | MTEYKLVVVGADGVGKSALTIQLIQNHFVDELLS |
| | 4)-(11-G12C-19)-(3- | VGGVGADGVGKLLSVGGMTEYKLVVVGACGVG |
| | G12C-4)-(11-G12V-19)- | KSALTIQLIQNHFVDELLSVGGVGACGVGKLLSV |
| | (3-G12V-4)-(11-G12R- | GGMTEYKLVVVGAVGVGKSALTIQLIQNHFVDEL |
| | 19)-(3-G12R-4)-(11- | LSVGGVGAVGVGKLLSVGGMTEYKLVVVGARGV |
| | G13C-19)-(3-G13C-4)- | GKSALTIQLIQNHFVDELLSVGGVGARGVGKLLS |
| | (11-G13D-19)-(3-G13D- | VGGTEYKLVVVGAGCVGKSALTIQLIQNHFVDEY |
| | 4)-(11-Q61H-19)-(3- | LLSVGGGAGCVGKSLLSVGGTEYKLVVVGAGDV |
| | Q61H-4)-(11-G13R-19)- | GKSALTIQLIQNHFVDEYLLSVGGGAGDVGKSLL |
| | (3-G13R-4)(SEQ ID NO: | SVGGTCLLDILDTAGHEEYSAMRDQYMRTGEGF |
| | 15) | LCLLSVGGTAGHEEYSLLSVGGTEYKLVVVGAG |
| | | RVGKSALTIQLIQNHFVDEYLLSVGGGAGRVGKS |
| | | (SEQ ID NO: 37) |
| 16 | (8-G12C-8)(8-G12D-8) | YKLVVVGACGVGKSALTYKLVVVGADGVGKSAL |
| | (8-G12V-8)(8-G12R-8) | TYKLVVVGAVGVGKSALTYKLVVVGARGVGKSA |
| | (8-G13C-8)(8-G13D-8) | LTKLVVVGAGCVGKSALTIKLVVVGAGDVGKSAL |
| | (8-Q61H-8)(8-G13R- | TILDILDTAGHEEYSAMRDKLVVVGAGRVGKSAL |
| | 8)(8-G12C-8)(8-G12D- | TIYKLVVVGACGVGKSALTYKLVVVGADGVGKSA |
| | 8)(8-G12V-8)(8-G12R- | LTYKLVVVGAVGVGKSALTYKLVVVGARGVGKS |
| | 8)(8-G13C-8)(8-G13D- | ALTKLVVVGAGCVGKSALTILDILDTAGHEEYSAM |
| | 8)(8-Q61H-8)(8-G13R- | RDKLVVVGAGDVGKSALTIKLVVVGAGRVGKSAL |
| | 8) | TIYKLVVVGAVGVGKSALTYKLVVVGARGVGKSA |
| | (8-G12C-8)(8-G12D-8) | LTKLVVVGAGCVGKSALTILDILDTAGHEEYSAMR |
| | (8-G12V-8)(8-G12R-8) | DYKLVVVGACGVGKSALTYKLVVVGADGVGKSA |
| | (8-G13C-8)(8-G13D-8) | LTKLVVVGAGDVGKSALTIKLVVVGAGRVGKSAL |
| | (8-Q61H-8)(8-G13R-8) | TI (SEQ ID NO: 38) |
| | (SEQ ID NO: 16) | |
| 17 | (11-G12D-19)-(3-G12D- | MTEYKLVVVGADGVGKSALTIQLIQNHFVDELLS |
| | 4)-(11-G12C-19)-(3- | VGGVGADGVGKLLSVGGMTEYKLVVVGACGVG |
| | G12C-4)-(11-G12V-19)- | KSALTIQLIQNHFVDELLSVGGVGACGVGKLLSV |
| | (3-G12V-4)-(11-G12R- | GGMTEYKLVVVGAVGVGKSALTIQLIQNHFVDEL |
| | 19)-(3-G12R-4)-(11- | LSVGGVGAVGVGKLLSVGGMTEYKLVVVGARGV |
| | G13C-19)-(3-G13C-4)- | GKSALTIQLIQNHFVDELLSVGGVGARGVGKLLS |
| | (11-G13D-19)-(3-G13D- | VGGTEYKLVVVGAGCVGKSALTIQLIQNHFVDEY |
| | 4)-(11-Q61H-19)-(3- | LLSVGGGAGCVGKSLLSVGGTEYKLVVVGAGDV |
| | Q61H-4)(SEQ ID NO: | GKSALTIQLIQNHFVDEYLLSVGGGAGDVGKSLL |
| | 17) | SVGGTCLLDILDTAGHEEYSAMRDQYMRTGEGF |
| | | LCLLSVGGTAGHEEYS (SEQ ID NO: 39) |
| 18 | (11-G12D-19)-(3-G12D- | MTEYKLVVVGADGVGKSALTIQLIQNHFVDELLS |
| | 4)-(11-G12C-19)-(3- | VGGVGADGVGKLLSVGGMTEYKLVVVGACGVG |
| | G12C-4)-(11-G12V-19)- | KSALTIQLIQNHFVDELLSVGGVGACGVGKLLSV |
| | (3-G12 V-4)-(11-G12R- | GGMTEYKLVVVGAVGVGKSALTIQLIQNHFVDEL |
| | 19)-(3-G12R-4)-(11- | LSVGGVGAVGVGKLLSVGGMTEYKLVVVGARGV |
| | Q61H-19)-(3-Q61H-4)- | GKSALTIQLIQNHFVDELLSVGGVGARGVGKLLS |
| | (11-G13D-19)-(3 -G13D- | VGGTCLLDILDTAGHEEYSAMRDQYMRTGEGFL |
| | 4)-(11-G12A-19)-(3- | CLLSVGGTAGHEEYSLLSVGGTEYKLVVVGAGD |
| | G12A-4)-(11-A146T-19)- | VGKSALTIQLIQNHFVDEYLLSVGGGAGDVGKSL |
| | (3-A146T-4)-(11-G13C- | LSVGGMTEYKLVVVGAAGVGKSALTIQLIQNHFV |
| | 19)-(3-G13C-4)-(11- | DELLSVGGVGAAGVGKLLSVGGRSYGIPFIETST |
| | G12S-19)-(3-G12S-4)- | KTRQRVEDAFYTLVREIRQLLSVGGETSTKTRQL |
| | (11-Q61R-19)-(3-Q61R- | LSVGGTEYKLVVVGAGCVGKSALTIQLIQNHFVD |
| | 4)(SEQ ID NO: 18) | EYLLSVGGGAGCVGKSLLSVGGMTEYKLVVVGA |
| | | SGVGKSALTIQLIQNHFVDELLSVGGVGASGVGK |
| | | LLSVGGTCLLDILDTAGREEYSAMRDQYMRTGE |
| | | GFLCLLSVGGTAGREEYS (SEQ ID NO: 40) |
| 19 | (11-G12C-19)-(3-G12C- | MTEYKLVVVGACGVGKSALTIQLIQNHFVDELLS |
| | 4)-(11-G12V-19)-(3- | VGGVGACGVGKLLSVGGMTEYKLVVVGAVGVG |
| | G12V-4)-(11-G12D-19)- | KSALTIQLIQNHFVDELLSVGGVGAVGVGKLLSV |
| | (3-G12D-4)-(11-Q61H- | GGMTEYKLVVVGADGVGKSALTIQLIQNHFVDEL |
| | 19)-(3-Q61H-4)-(11- | LSVGGVGADGVGKLLSVGGTCLLDILDTAGHEEY |
| | G13C-19)-(3-G13C-4)- | SAMRDQYMRTGEGFLCLLSVGGTAGHEEYSSLL |
| | (11-G13D-19)-(3-G13D- | SVGGTEYKLVVVGAGCVGKSALTIQLIQNHFVDE |
| | 4)-(11-G12A-19)-(3- | YLLSVGGGAGCVGKSLLSVGGTEYKLVVVGAGD |
| | G12A-4)-(11-G12S-19)- | VGKSALTIQLIQNHFVDEYLLSVGGGAGDVGKSL |
| | (3-G12S-4)(SEQ ID NO: | LSVGGMTEYKLVVVGAAGVGKSALTIQLIQNHFV |
| | 19) | DELLSVGGVGAAGVGKLLSVGGMTEYKLVVVGA |
| | | SGVGKSALTIQLIQNHFVDELLSVGGVGASGVGK |
| | | (SEQ ID NO: 41) |
| 20 | (11-G12D-19)-(3-G12D- | MTEYKLVVVGADGVGKSALTIQLIQNHFVDELLS |
| | 4)-(11-G12V-19)-(3- | VGGVGADGVGKLLSVGGMTEYKLVVVGAVGVG |
| | G12V-4)-(11-G12R-19)- | KSALTIQLIQNHFVDELLSVGGVGAVGVGKLLSV |
| | (3-G12R-4)-(11-Q61H- | GGMTEYKLVVVGARGVGKSALTIQLIQNHFVDEL |
| | 19)-(3-Q61H-4)-(11- | LSVGGVGARGVGKLLSVGGTCLLDILDTAGHEEY |
| | Q61R-19)-(3-Q61R-4)- | SAMRDQYMRTGEGFLCLLSVGGTAGHEEYSSLL |
| | (11-G13C-19)-(3-G13C- | SVGGTCLLDILDTAGREEYSAMRDQYMRTGEGF |
| | 4)-(11-G12C-19)-(3- | LCLLSVGGTAGREEYSLLSVGGTEYKLVVVGAG |
| | G12C-4)-(11-G13D-19)- | CVGKSALTIQLIQNHFVDEYLLSVGGGAGCVGKS |
| | (3-G13D-4)(SEQ ID NO: | LLSVGGMTEYKLVVVGACGVGKSALTIQLIQNHF |
| | 20) | VDELLSVGGVGACGVGKLLSVGGTEYKLVVVGA |
| | | GDVGKSALTIQLIQNHFVDEYLLSVGGGAGDVGK |
| | | S (SEQ ID NO: 42) |
| 21 | (11-G12D-19)-(3-G12D- | MTEYKLVVVGADGVGKSALTIQLIQNHFVDELLS |
| | 4)-(11-G12V-19)-(3- | VGGVGADGVGKLLSVGGMTEYKLVVVGAVGVG |
| | G12V-4)-(11-G13D-19)- | KSALTIQLIQNHFVDELLSVGGVGAVGVGKLLSV |
| | (3-G13D-4)-(11-A146T- | GGTEYKLVVVGAGDVGKSALTIQLIQNHFVDEYL |
| | 19)-(3-A146T-4)-(11- | LSVGGGAGDVGKSLLSVGGRSYGIPFIETSTKTR |
| | G12C-19)-(3-G12C-4)- | QRVEDAFYTLVREIRQLLSVGGETSTKTRQLLSV |
| | (11-G12A-19)-(3-G12A- | GGMTEYKLVVVGACGVGKSALTIQLIQNHFVDEL |
| | 4)-(11-G12S-19)-(3- | LSVGGVGACGVGKLLSVGGMTEYKLVVVGAAGV |
| | G12S-4)-(11-G13C-19)- | GKSALTIQLIQNHFVDELLSVGGVGAAGVGKLLS |
| | (3-G13C-4)(SEQ ID NO: | VGGMTEYKLVVVGASGVGKSALTIQLIQNHFVDE |
| | 21) | LLSVGGVGASGVGKLLSVGGTEYKLVVVGAGCV |
| | | GKSALTIQLIQNHFVDEYLLSVGGGAGCVGKS |
| | | (SEQ ID NO: 43) |
| 22 | (11-G12D-19)-(3-G12D- | MTEYKLVMVGADGVGKSALTIQLIQNHFVDELLS |
| | 4)-(11-G12C-19)-(3- | VGGVGADGVGKLLSVGGMTEYKLVVVGACGVG |
| | G12C-4)-(11-G12V-19)- | KSALTIQLIQNHFVDELLSVGGVGACGVGKLLSV |
| | (3-G12V-4)-(11-G12R- | GGMTEYKLVVVGAVGVGKSALTIQLIQNHFVDEL |
| | 19)-(3-G12R-4)-(11- | LSVGGVGAVGVGKLLSVGGMTEYKLVVVGARGV |
| | G13C-19)-(3-G13C-4)- | GKSALTIQLIQNHFVDELLSVGGVGARGVGKLLS |
| | (11-G13D-19)-(3-G13D- | VGGTEYKLVVVGAGCVGKSALTIQLIQNHFVDEY |
| | 4)-(11-Q61H-19)-(3- | LLSVGGGAGCVGKSLLSVGGTEYKLVVVGAGDV |
| | Q61H-4)(SEQ ID NO: | GKSALTIQLIQNHFVDEYLLSVGGGAGDVGKSLL |
| | 22) | SVGGTCLLDILDTAGHEEYSAMRDQYMRTGEGF |
| | | LCLLSVGGTAGHEEYS (SEQ ID NO: 44) |

Open reading frames (ORFs) encoding the antigenic peptides listed in Table 1 (SEQ ID NOs: 23 to 44) were synthesized with the 5′-UTR and/or a signal sequence linked thereto. The synthesized sequences were designed to include HindIII and Xhol restriction enzyme sites at the 5′ end and 3′ end, respectively, to allow for cloning. Each synthesized sequence encoding the antigens was digested with HindIII and Xhol along with a template vector, and ligated using DNA ligase to generate a vector including a sequence encoding “mRNA.” The KRAS variant peptide compositions of Antigens 1 to 22 are as shown in Table 1. Each variant peptide is separated by parentheses, and the presence of linkers between variant peptides is indicated by a hyphen (“-”). The linker indicated by a hyphen represents a cathepsin cleavage linker having the amino acid sequence ‘LLSVGG’ (SEQ ID NO: 553). In antigenic sequences where no hyphen is present and the variant peptides are linked in sequence order, this indicates that the variant peptides are directly linked without a linker.

The full-length mRNAs encoding antigens 1 to 22 shown in Table 1 each have the structure of 5′UTR-ORF-stop codon-3′UTR-polyA. Table 2 shows the variant peptides contained in Antigen 1 and the mRNA sequence thereof.

TABLE 2

| Peptide | Length | | |
|---|---|---|---|
| name | (aa) | mRNA sequence | Peptide |
| 11-G12R-11 | 23 | AUGACAGAGUACAAGCUGGGU | MTEYKLVVVGARGVGKS |
| | | GGUCGUGGGGGCCAGGGGCG | ALTIQL (SEQ ID NO: |
| | | UGGGCAAGAGCGCCCUGACUA | 301) |
| | | UCCAGCUG (SEQ ID NO: 45) | |
| 9-G12D-9 | 19 | GAAUACAAGCUGGUGGUGGUG | EYKLVVVGADGVGKSAL |
| | | GGCGCCGACGGCGUGGGGAA GUCCGCCCUCACAAUC (SEQ ID | TI (SEQ ID NO: 302) |
| | | NO: 46) | |
| 9-G12V-9 | 19 | GAGUACAAGCUGGUCGUGGUG | EYKLVVVGAVGVGKSAL |
| | | GGGGCCGUGGGCGUCGGGAA | TI (SEQ ID NO: 303) |
| | | GUCCGCCCUGACAAUC (SEQ | |
| | | ID NO: 47) | |
| 9-G12C-9 | 19 | GAGUACAAGUUGGUGGUGGUG | EYKLVVVGACGVGKSAL |
| | | GGCGCCUGCGGGGUGGGCAA | TI (SEQ ID NO: 304) |
| | | GAGCGCCCUGACCAUC (SEQ | |
| | | ID NO: 48) | |
| 9-G13D-9 | 19 | UACAAGCUCGUGGUGGUGGG | YKLVVVGAGDVGKSALTI |
| | | GGCCGGGGACGUGGGGAAGA | Q (SEQ ID NO: 305) |
| | | GUGCCCUGACCAUCCAG (SEQ | |
| | | ID NO: 49) | |
| 9-G13C-9 | 19 | UAUAAGCUGGUGGUGGUCGGC | YKLVVVGAGCVGKSALTIQ |
| | | GCCGGGUGCGUGGGCAAGUC | (SEQ ID NO: 306) |
| | | CGCCCUGACAAUCCAG (SEQ ID | |
| | | NO: 50) | |
| 9-Q61H-9 | 19 | CUGCUGGAUAUCCUGGGAAUA | LLDILDTAGHEEYSAMR |
| | | CCGCCGGCCACGAGGAGUACA | DQ (SEQ ID NO: 307) |
| | | GCGCCAUGAGGGAUCAG (SEQ | |
| | | ID NO: 51) | |

Table 3 shows the variant peptides contained in Antigen 2 and their mRNA sequences.

TABLE 3

| Peptide | Length | | |
|---|---|---|---|
| name | (aa) | mRNA sequence | Peptide |
| 8-G12C-8 | 17 | UACAAGCUGGUCGUCGUGGGG | YKLVVVGACGVGKSALT |
| | | GCCUGCGGCGUGGGGAAGUC | (SEQ ID NO: 308) |
| | | CGCUCUGACC (SEQ ID NO: 52) | |
| 8-G12D-8 | 17 | UACAAACUGGUGGUGGUGGGG | YKLVVVGADGVGKSALT |
| | | GCCGAUGGGGUGGGCAAGUC | (SEQ ID NO: 309) |
| | | CGCCCUGACC (SEQ ID NO: 53) | |
| 8-G12V-8 | 17 | UACAAGCUGGGUGGUGGUCG | YKLVVVGAVGVGKSALT |
| | | GGGCCGUCGGCGUGGGCAAAA | (SEQ ID NO: 310) |
| | | GCGCCCUGACA (SEQ ID NO: | |
| | | 54) | |
| 8-G12R-8 | 17 | UACAAGCUCGUGGUGGUGGGC | YKLVVVGARGVGKSALT |
| | | GCCAGGGGGGUGGGCAAGUC | (SEQ ID NO: 311) |
| | | CGCCCUGACA (SEQ ID NO: 55) | |
| 8-G13C-8 | 17 | AAGCUGGUCGUGGUGGGCGC | KLVVVGAGCVGKSALTI |
| | | CGGGUGCGUGGGCAAGAGCG | (SEQ ID NO: 312) |
| | | CCCUGACAAUC(SEQ ID NO: 56) | |
| 8-G13D-8 | 17 | AAGCUGGUGGUGGUGGGGGC | KLVVVGAGDVGKSALTI |
| | | CGGGGAUGUGGGCAAGAGCG | (SEQ ID NO: 313) |
| | | CCUGACCAUC (SEQ ID NO: 57) | |
| 8-Q61H-8 | 17 | CUGGACAUCCUGGACACAGCC | LDILDTAGHEEYSAMRD |
| | | GGGCACGAGGAGUACAGCGCC | (SEQ ID NO: 314) |
| | | AUGAGGGAU (SEQ ID NO: 58) | |

Table 4 shows the variant peptides contained in Antigen 3 and their mRNA sequences.

TABLE 4

| Peptide | Length | | |
|---|---|---|---|
| name | (aa) | mRNA sequence | Peptide |
| 8-G12C-8 | 17 | UACAAGCUCGUGGUGGUGGGC | YKLVVVGACGVGKSALT |
| | | GCCUGCGGCGUGGGGAAGUC | (SEQ ID NO: 315) |
| | | CGCCCUGACA (SEQ ID NO: 59) | |
| 8-G12D-8 | 17 | UACAAGCUGGUGGUCGUGGGC | YKLVVVGADGVGKSALT |
| | | GCCGACGGGGUGGGCAAGAG | (SEQ ID NO: 316) |
| | | CGCCCUCACC (SEQ ID NO: 60) | |
| 8-G12V-8 | 17 | UACAAGCUGGUCGUGGUCGGG | YKLVVVGAVGVGKSALT |
| | | GCCGUGGGCGUGGGCAAGUC | (SEQ ID NO: 317) |
| | | CGCUCUGACA (SEQ ID NO: 61) | |
| 8-G12R-8 | 17 | UACAAGCUGGGUGGUGGUGG | YKLVVVGARGVGKSALT |
| | | GGGCCAGGGGGGUCGGGAAA | (SEQ ID NO: 318) |
| | | UCCGCCCUGACU (SEQ ID NO: | |
| | | 62) | |
| 8-G13C-8 | 17 | AAGCUCGUGGUGGUGGGGGC | KLVVVGAGCVGKSALTI |
| | | CGGGUGCGUGGGGAAGUCCG | (SEQ ID NO: 319) |
| | | CCCUGACAUU (SEQ ID NO: 63) | |
| 8-G13D-8 | 17 | AAGCUGGUCGUGGUGGGCGC | KLVVVGAGDVGKSALTI |
| | | CGGGGAUGUGGGCAAGAGCG | (SEQ ID NO: 320) |
| | | CCUGACCAUC (SEQ ID NO: 64) | |
| 8-Q61H-8 | 17 | CUGGACAUCCUGGGACACCGC | ILDILDTAGHEEYSAMRD |
| | | CGGCCACGAGGAGUACAGCGC | (SEQ ID NO: 321) |
| | | CAUGAGGGAU (SEQ ID NO: 65) | |

Table 5 shows the variant peptides contained in Antigen 4 and their mRNA sequences.

TABLE 5

| Peptide | Length | | |
|---|---|---|---|
| name | (aa) | mRNA sequence | Peptide |
| 3-G12C-5 | 9 | GUUGGAGCCUGUGGAGUUGG | VGACGVGKS (SEQ ID |
| | | UAAAAGU (SEQ ID NO: 66) | NO: 322) |
| 5-G12C-3 | 9 | GUAGUUGUGGGAGCUUGUGG | VVVGACGVG |
| | | AGUAGGU | (sequence |
| | | (SEQ ID NO: 67) | number 323) |
| 3-G12C-5 | 9 | GUAGGAGCUUGUGGAGUAGGA | VGACGVGKS (SEQ ID |
| | | AAGUCU (SEQ ID NO: 68) | NO: 324) |
| 5-G12C-3 | 9 | GUAGUAGUAGGAGCAUGCGGA | VVVGACGVG (SEQ ID |
| | | GUCGGA (SEQ ID NO: 69) | NO: 325) |
| 3-G12C-5 | 9 | GUAGGAGCAUGCGGAGUAGGA | VGACGVGKS (SEQ ID |
| | | AAGUCA (SEQ ID NO: 70) | NO: 326) |
| 5-G12C-3 | 9 | GUAGUAGUAGGAGCUUGUGGA | VVVGACGVG (SEQ ID |
| | | GUAGGA (SEQ ID NO: 71) | NO: 327) |

Table 6 shows the variant peptides contained in Antigen 5 and their mRNA sequences.

TABLE 6

| Peptide | Length | | |
|---|---|---|---|
| name | (aa) | mRNA sequence | Peptide |
| 3-G12C-5 | 9 | GUGGGCGCCUGUGGAGUAGG | VGACGVGKS (SEQ ID |
| | | AAAAUCU (SEQ ID NO: 72) | NO: 328) |
| 5-G12C-3 | 9 | GUGGUGGUCGGAGCAUGCGG | VVVGACGVG (SEQ ID |
| | | AGUAGGA (SEQ ID NO: 73) | NO: 329) |
| 3-G12D-5 | 9 | GUGGGAGCUGACGGCGUGGG | VGADGVGKS (SEQ ID |
| | | UAAAUCC (SEQ ID NO: 74) | NO: 330) |
| 5-G12D-3 | 9 | GUGGUCGUGGGAGCUGACGG | VVVGADGVG (SEQ ID |
| | | UGUUGGU (SEQ ID NO: 75) | NO: 331) |
| 3-G13D-5 | 9 | GGAGCGGGAGAUGUUGGAAAG | GAGDVGKSA (SEQ ID |
| | | UCAGCC (SEQ ID NO: 76) | NO: 332) |
| 5-G13D-3 | 9 | GUUGUGGGGGCUGGUGACGU | VVGAGDVGK (SEQ ID |
| | | GGGAAAG (SEQ ID NO: 77) | NO: 333) |

Table 7 shows the variant peptides contained in Antigen 6 and their mRNA sequences.

TABLE 7

| Peptide | Length | | |
|---|---|---|---|
| name | (aa) | mRNA sequence | Peptide |
| 11-G12C-19 | 31 | AUGACAGAAUACAAACUUGUGGU | MTEYKLVVVGACGVGKS |
| | | AGUUGGAGCAUGCGGUGUGGGU | ALTIQLIQNHFVDE (SEQ |
| | | AAGAGUGCCCUCACCAUCCAACU | ID NO: 334) |
| | | AAUUCAGAACCACUUCGUUGACG | |
| | | AA (SEQ ID NO: 78) | |
| 3-G12C-4 | 8 | GUUGGAGCAUGUGGUGUAGGGA | VGACGVGK (SEQ ID |
| | | AG (SEQ ID NO: 79) | NO: 335) |
| 11-G12D-19 | 31 | AUGACUGAAUACAAACUUGUGGU | MTEYKLVVVGADGVGKS |
| | | UGUUGGAGCAGAUGGGGUCGGA | ALTIQLIQNHFVDE (SEQ |
| | | AAAUCCGCCUUGACAAUACAGCU | ID NO: 336) |
| | | UAUUCAGAAUCACUUUGUGGAUG | |
| | | AG (SEQ ID NO: 80) | |
| 3-G12D-4 | 8 | GUCGGAGCUGAUGGCGUCGGAA | VGADGVGK (SEQ ID |
| | | AG (SEQ ID NO: 81) | NO: 337) |
| 11-G13D-19 | 31 | ACUGAGUAUAAAUUAGUGGUAGU | TEYKLVVVGAGDVGKSA |
| | | CGGGGCUGGAGACGUGGGCAAG | LTIQLIQNHFVDEY (SEQ |
| | | AGUGCCCUUACCAUCCAGCUCAU | ID NO: 338) |
| | | CCAGAAUCAUUUUGUGGACGAGU | |
| | | AU (SEQ ID NO: 82) | |
| 3-G13D-4 | 8 | GGAGCUGGUGACGUCGGAAAGU | GAGDVGKS (SEQ ID |
| | | CU (SEQ ID NO: 83) | NO: 339) |

Table 8 shows the variant peptides contained in Antigen 7 and their mRNA sequences.

TABLE 8

| Peptide | Length | | |
|---|---|---|---|
| name | (aa) | mRNA sequence | Peptide |
| 12-G13C-18 | 31 | AUGACUGAAUAUAAACUCGUC | MTEYKLVVVGAGCVGKS |
| | | GUGGUUGGAGCAGGUUGCGU | ALTIQLIQNHFVDE (SEQ |
| | | UGGCAAAAGCGCCCUUACAAU | ID NO: 340) |
| | | CCAGCUGAUACAGAACCACUU | |
| | | CGUGGAUGAG (SEQ ID NO: 84) | |
| 11-G12C-19 | 31 | AUGACAGAGUACAAGUUGGUG | MTEYKLVVVGACGVGKS |
| | | GUGGUGGGCGCCUGUGGGGU | ALTIQLIQNHFVDE (SEQ |
| | | GGGGGAAAUCUGCCCUUACUA | ID NO: 341) |
| | | UCCAGCUGAUCCAGAACCACU | |
| | | UUGUGGAUGAG (SEQ ID NO: | |
| | | 85) | |
| 11-G12D-19 | 31 | AUGACAGAAUACAAACUGGUC | MTEYKLVVVGADGVGKS |
| | | GUGGUCGGCGCUGAUGGCGU | ALTIQLIQNHFVDE (SEQ |
| | | UGGGAAGAGUGCUCUGACAAU | ID NO: 342) |
| | | UCAACUCAUCCAGAAUCACUU | |
| | | CGUAGACGAA (SEQ ID NO: 86) | |
| 12-G13D-18 | 31 | AUGACGGAGUAUAAGCUCGUU | MTEYKLVVVGAGDVGKS |
| | | GUGGUAGGCGCCGGAGACGUA | ALTIQLIQNHFVDE (SEQ |
| | | GGCAAGUCCGCACUGACCAUU | ID NO: 343) |
| | | CAGCUCAUUCAAAAUUCAUUU | |
| | | UGUAGACGAA (SEQ ID NO: 87) | |
| 11-G12V-19 | 31 | AUGACCGAGUACAAGCUGGUU | MTEYKLVVVGAVGVGKS |
| | | GUUGUGGGUGCCGUGGGGGU | ALTIQLIQNHFVDE (SEQ |
| | | GGGUAAGUCAGCCCUCACCAU | ID NO: 344) |
| | | UCAGCUCAUCCAAAACCAUUU | |
| | | CGUGGACGAG (SEQ ID NO: 88) | |
| 11-Q61H-19 | 31 | ACCUGCCUCCUGGACAUAUUG | TCLLDILDTAGHEEYSAM |
| | | GACACUGCGGGGCAUGAGGAG | RDQYMRTGEGFLC |
| | | UAUUCCGCAAUGCGCGAUCAG | (SEQ ID NO: 345) |
| | | UACAUGAGGACCGGAGAAGGA | |
| | | UUUCUGUGC (SEQ ID NO: 89) | |

Table 9 shows the variant peptides contained in Antigen 8 and their mRNA sequences.

TABLE 9

| Peptide | Length | | |
|---|---|---|---|
| name | (aa) | mRNA sequence | Peptide |
| 11-G12C-19 | 31 | AUGACAGAGUAUAAGCUGGUC | MTEYKLVVVGACGVGKS |
| | | GUGGUCGGCGCGUGCGGAGU | ALTIQLIQNHFVDE (SEQ |
| | | GGGGAAAUCAGCUCUCACCAU | ID NO: 346) |
| | | UCAGCUAAUCCAGAACCACUU | |
| | | CGUUGACGAA (SEQ ID NO: 90) | |
| 3-G12C-4 | 8 | GUCGGCGCUUGCGGUGUCGG | VGACGVGK (SEQ ID |
| | | AAAG (SEQ ID NO: 91) | NO: 347) |
| 11-G12D-19 | 31 | AUGACGGAGUAUAAACUUGUC | MTEYKLVVVGADGVGKS |
| | | GUAGUUGGCGCCGACGGUGU | ALTIQLIQNHFVDE (SEQ |
| | | UGGAAAAUCCGCAUUAACCAU | ID NO: 348) |
| | | ACAAUUGAUCCAGAACCACUU | |
| | | CGUGGACGAA (SEQ ID NO: 92) | |
| 3-G12D-4 | 8 | GUGGGGGCUGACGGCGUAGG | VGADGVGK (SEQ ID |
| | | AAAA (SEQ ID NO: 93) | NO: 349) |
| 11-G13D-19 | 31 | ACAGAGUACAAGCUGGUGGUG | TEYKLVVVGAGDVGKSA |
| | | GUUGGCGCAGGGGAUGUGGG | LTIQLIQNHFVDEY (SEQ |
| | | UAAGAGCGCCCUGACUAUUCA | ID NO: 350) |
| | | GCUGAUCCAAAAUCAUUUUGU | |
| | | GGAUGAAUAC (SEQ ID NO: 94) | |
| 3-G13D-4 | 8 | GGAGCAGGCGAUGUGGGGAA | GAGDVGKS (SEQ ID |
| | | GUCU (SEQ ID NO: 95) | NO: 351) |

Table 10 shows the N-terminal domain, variant peptides and their mRNA sequences contained in Antigen 9.

TABLE 10

| Peptide | Length | | |
|---|---|---|---|
| name | (aa) | mRNA sequence | Peptide |
| DTT | 177 | UGUAUAAACCUCGAUUGGGAU | CINLDWDVIRDKTKTKIE |
| | | GUAAUCCGCGACAAAACCAAG | SLKEHGPIKNKMSESPN |
| | | ACAAAAAUUGAAUCUCUCAAGG | KTVSEEKAKQYLEEFHQ |
| | | AGCACGGCCCUAUCAAAAAUAA | TALEHPELSELKTVTGTN |
| | | AAUGUCAGAGUCACCCAAUAA | PVFAGANYAAWAVNVA |
| | | GACGGUGUCUGAGGAAAAGGC | QVIDSETADNLEKTTAAL |
| | | CAAGCAAUACUUGGAAGAAUU | SILPGIGSVMGIADGAVH |
| | | UCAUCAAACCGCCUUGGAACA | HNTEEIVAQSIALSSLMV |
| | | UCCGGAACUAAGCGAGCUUAA | AQAIPLVGELVDIGFAAY |
| | | GACAGUUACAGGUACUAACCC | NFVESIINLFQVVHNSYN |
| | | AGUGUUUGCCGGAGCUAACUA | R (SEQ ID NO: 352) |
| | | CGCCGCGUGGGCGGUCAAUG | |
| | | UAGCCCAGGUCAUUGAUAGUG | |
| | | AAACUGCUGACAAUUUAGAAAA | |
| | | GACAACUGCCGCUCUCUCCAU | |
| | | UCUGCCCGGAAUAGGGAGCGU | |
| | | UAUGGGUAUCGCAGAUGGCGC | |
| | | GGUGCAUCAUAACACCGAGGA | |
| | | GAUCGUCGCACAGUCGAUCGC | |
| | | UCUCAGUAGCCUAAUGGUGGC | |
| | | UCAGGCAAUUCCUCUGGUCGG | |
| | | GGAACUGGUUGACAUCGGAUU | |
| | | CGCCGCAUACAAUUUCGUAGA | |
| | | GUCUAUUAUAAAUCUUUUCCA | |
| | | GGUGGUCCACAACUCAUAUAA | |
| | | UCGA (SEQ ID NO: 96) | |
| 12-G13C-18 | 31 | AUGACUGAGUACAAGCUGGUA | MTEYKLVVVGAGCVGKS |
| | | GUGGUAGGGGCUGGAUGCGU | ALTIQLIQNHFVDE (SEQ |
| | | CGGGAAGUCAGCCCUGACUAU | ID NO: 353) |
| | | ACAGCUGAUCCAGAACCACUU | |
| | | CGUGGAUGAG (SEQ ID NO: 97) | |
| 11-G12C-19 | 31 | AUGACGGAAUAUAAGCUCGUU | MTEYKLVVVGACGVGKS |
| | | GUGGUAGGGGCUUGUGGAGU | ALTIQLIQNHFVDE (SEQ |
| | | GGGCAAGUCCGCACUGACAAU | ID NO: 354) |
| | | CCAGCUGAUCCAGAAUCACUU | |
| | | UGUGGAUGAG (SEQ ID NO: 98) | |
| 11-G12D-19 | 31 | AUGACCGAAUACAAACUUGUU | MTEYKLVVVGADGVGKS |
| | | GUGGUGGGCGCUGACGGAGU | ALTIQLIQNHFVDE (SEQ |
| | | UGGUAAAAGCGCCCUUACCAU | ID NO: 355) |
| | | CCAACUGAUUCAGAACCACUU | |
| | | UGUCGACGAG (SEQ ID NO: 99) | |
| 12-G13D-18 | 31 | AUGACCGAGUAUAAAUUGGUG | MTEYKLVVVGAGDVGKS |
| | | GUGGUGGGCGCUGGCGACGU | ALTIQLIQNHFVDE (SEQ |
| | | GGGCAAAAGUGCACUGACAAU | ID NO: 356) |
| | | UCAGCUGAAUUCAGAACCACU | |
| | | UUGUGGACGAG (SEQ ID NO: | |
| | | 100) | |
| 11-G12V-19 | 31 | AUGACCGAGUAUAAGUUGGUG | MTEYKLVVVGAVGVGKS |
| | | GUUGUCGGCGCGGUUGGUGU | ALTIQLIQNHFVDE (SEQ |
| | | GGGAAAAUCUGCCCUUACAAU | ID NO: 357) |
| | | CCAGCUGAUUCAAAACCAUUU | |
| | | CGUCGACGAG (SEQ ID NO: | |
| | | 101) | |
| 11-Q61H-19 | 31 | ACGUGCUUGCUCGACAUCCUG | TCLLDILDTAGHEEYSAM |
| | | GAUACUGCCGGCCACGAAGAG | RDQYMRTGEGFLC |
| | | UACUCCGCCAUGCGGGAUCAA | (SEQ ID NO: 358) |
| | | UAUAUGCGUACCGGCGAGGGG | |
| | | UUCUUAUGU (SEQ ID NO: 102) | |

Antigen 9 is a peptide in which the DTT domain (1-177aa) and six variant peptides described in Table 10 are sequentially linked with a linker. Table 11 shows the variant peptides included in Antigen 10, the C-terminal domain, and its mRNA sequence.

TABLE 11

| Peptide | Length | | |
|---|---|---|---|
| name | (aa) | mRNA sequence | Peptide |
| 12-G13C-18 | 31 | AUGACAGAGUACAAGCUGGUG | MTEYKLVVVGAGCVGKS |
| | | GUCGUCGGAGCCGGUUGCGU | ALTIQLIQNHFVDE |
| | | AGGAAAGAGCGCAUUAACCAU | (SEQ |
| | | CCAGCUAAUCCAAAACCAUUUC | ID NO: 359) |
| | | GUGGACGAA | |
| | | (SEQ ID NO: 103) | |
| 11-G12C-19 | 31 | AUGACUGAAUACAAGUUAGUG | MTEYKLVVVGACGVGKS |
| | | GUGGUUGGGGCCUGUGGUGU | ALTIQLIQNHFVDE |
| | | GGGAAAAUCCGCUCUCACGAU | (SEQ |
| | | CCAGCUGAUUCAGAACCACUU | ID NO: 360) |
| | | CGUCGACGAG | |
| | | (SEQ ID NO: | |
| | | 104) | |
| 11-G12D-19 | 31 | AUGACCGAAUAUAAACUGGUU | MTEYKLVVVGADGVGKS |
| | | GUCGUGGGGGGCUGAUGGAG | ALTIQLIQNHFVDE |
| | | UUGGCAAGUCUGCCUUGACUA | (SEQ |
| | | UUCAACUGAUCCAGAAUUCAU | ID NO: 361) |
| | | UUUGUGGAUGAG | |
| | | (SEQ ID NO: | |
| | | 105) | |
| 12-G13D-18 | 31 | AUGACAGAGUAUAAACUCGUC | MTEYKLVVVGAGDVGKS |
| | | GUGGUGGGUGCCGGAGAUGU | ALTIQLIQNHFVDE |
| | | UGGGAAAUCUGCUCUUACCAU | (SEQ |
| | | ACAGUUAAUCCAAAAUCACUUC | ID NO: 362) |
| | | GUUGAUGAA | |
| | | (SEQ ID NO: 106) | |
| 11-G12V-19 | 31 | AUGACUGAGUACAAGUUGGUG | MTEYKLVVVGAVGVGKS |
| | | GUGGUGGGGGCUGUGGGGGU | ALTIQLIQNHFVDE |
| | | GGGAAAGUCUGCACUGACUAU | (SEQ |
| | | UCAGCUCAUUCAGAACCACUU | ID NO: 363) |
| | | CGUGGAUGAG (SEQ ID NO: | |
| | | 107) | |
| 11-Q61H-19 | 31 | ACCUGUCUGCUAGACAUUCUG | TCLLDILDTAGHEEYSAM |
| | | GACACGGCCGGCCAUGAGGAG | RDQYMRTGEGFLC |
| | | UAUAGCGCAAUGAGAGACCAA | (SEQ ID NO: 364) |
| | | UACAUGCGGACCGGCGAAGGC | |
| | | UUUCUUUGC | |
| | | (SEQ ID NO: 108) | |
| DTT | 177 | UGUAUAAACCUCGACUGGGAU | CINLDWDVIRDKTKTKIE |
| | | GUGAUCCGCGAUAAAACCAAG | SLKEHGPIKNKMSESPN |
| | | ACCAAGAUCGAAAGCCUAAAG | KTVSEEKAKQYLEEFHQ |
| | | GAGCAUGGCCCUAUUAAGAAU | TALEHPELSELKTVTGTN |
| | | AAAAUGUCAGAAUCUCCCAAUA | PVFAGANYAAWAVNVA |
| | | AAACAGUAUCCGAGGAAAAGG | QVIDSETADNLEKTTAAL |
| | | CCAAACAGUAUUUGGAAGAGU | SILPGIGSVMGIADGAVH |
| | | UUCACCAGACCGCCCUGGAAC | HNTEEIVAQSIALSSLMV |
| | | ACCCAGAGCUGUCCGAACUGA | AQAIPLVGELVDIGFAAY |
| | | AGACAGUCACUGGGACAAAUC | NFVESIINLFQVVHNSYNR |
| | | CUGUCUUCGCCGGUGCUAACU | (SEQ ID NO: 365) |
| | | ACGCGGCAUGGGCGGUCAAUG | |
| | | UAGCCCAAGUUAUCGAUAGUG | |
| | | AGACUGCCGACAACCUUGAGA | |
| | | AAACCACAGCAGCAUUGAGCA | |
| | | UAUUGCCGGGCAUCGGUUCUG | |
| | | UCAUGGGCAUAGCUGACGGCG | |
| | | CUGUGCACCACAACACAGAGG | |
| | | AGAUCGUCGCUCAGAGUAUUG | |
| | | CGCUGUCCAGUCUUAUGGUGG | |
| | | CCCAGGCCAUCCCACUUGUUG | |
| | | GUGAACUGGUGGACAUCGGAU | |
| | | UUGCAGCGUACAACUUUGUCG | |
| | | AGUCAAUUAUUAAUCUGUUCC | |
| | | AGGUUGUCCAUAACAGUUAUA | |
| | | AUCGU (SEQ ID NO: 109) | |

Antigen 10 is a peptide in which the six variant peptides and the DTT domain (1-177aa) described in Table 11 are sequentially linked together with linkers. Table 12 shows the N-terminal domain, variant peptides, C-terminal domain, and mRNA sequences thereof included in Antigen 11.

TABLE 12

| Peptide | Length | | |
|---|---|---|---|
| name | (aa) | mRNA sequence | Peptide |
| DTT(1-87) | 87 | UGUAUAAAUCUUGACUGGGAC | CINLDWDVIRDKTKTKIE |
| | | GUCAUUAGGGAUAAGACAAAG | SLKEHGPIKNKMSESPN |
| | | ACCAAGAUAGAGUCCUUGAAG | KTVSEEKAKQYLEEFHQ |
| | | GGAGCACGGCCCCAUCAAAAA | TALEHPELSELKTVTGTN |
| | | CAAAAUGUCGGAGAGCCCAAA | PVFAGANYAAWAVNVA |
| | | CAAAACAGUGAGUGAAGAGAA | Q (SEQ ID NO: 366) |
| | | AGCAAAGCAGUAUCUUGAAGA | |
| | | GUUUCACCAGACGGCUCUGGA | |
| | | ACAUCCUGAGCUGUCUGAACU | |
| | | UAAGACAGUGACCGGCACAAA | |
| | | CCCUGUGUUUGCAGGAGCUAA | |
| | | UUAUGCGCUUGGGCCGUUAAC | |
| | | GUGGCUCAG (SEQ ID NO: 110) | |
| 12-G13C-18 | 31 | AUGACUGAGUAUAAACUGGUU | MTEYKLVVVGAGCVGKS |
| | | GUGGUGGGCGCCGGCUGCGU | ALTIQLIQNHFVDE (SEQ |
| | | CGGAAAGAGCGCGCUGACCAU | ID NO: 367) |
| | | UCAACUUAUUCAAAAUCACUUC | |
| | | GUUGAUGAA (SEQ ID NO: 111) | |
| 11-G12C-19 | 31 | AUGACUGAAUAUAAGUUAGUG | MTEYKLVVVGACGVGKS |
| | | GUCGUGGGCGCCUGUGGGGU | ALTIQLIQNHFVDE (SEQ |
| | | GGGUAAAAGCGCCCUAACUAU | ID NO: 368) |
| | | ACAGCUAAUCCAGAACCACUU | |
| | | UGUAGACGAG (SEQ ID NO: | |
| | | 112) | |
| 11-G12D-19 | 31 | AUGACAGAGUACAAGCUGGGU | MTEYKLVVVGADGVGKS |
| | | GGUUGUAGGUGCAGACGGCG | ALTIQLIQNHFVDE (SEQ |
| | | UCGGCAAAUCCGCCCUCACUA | ID NO: 369) |
| | | UCCAGCUGAUUCAGAACCACU | |
| | | UUGUAGACGAG (SEQ ID NO: | |
| | | 113) | |
| 12-G13D-18 | 31 | AUGACUGAAUACAAACUUGUU | MTEYKLVVVGAGDVGKS |
| | | GUGGUUGGGGCUGGGGAUGU | ALTIQLIQNHFVDE (SEQ |
| | | CGGAAAGAGUGCUCUGACGAU | ID NO: 370) |
| | | UCAGCUGAUCCAGAACCAUUU | |
| | | UGUGGACGAA (SEQ ID NO: | |
| | | 114) | |
| 11-G12V-19 | 31 | AUGACCGAGUACAAGCUGGGU | MTEYKLVVVGAVGVGKS |
| | | GGUUGUGGGAGCGGUCGGGG | ALTIQLIQNHFVDE (SEQ |
| | | UCGGGAAGUCCGCCUUAACAA | ID NO: 371) |
| | | UCCAGCUGAUCCAAAAUCACU | |
| | | UCGUGGGAUGAA (SEQ ID NO: | |
| | | 115) | |
| 11-Q61H-19 | 31 | ACCUGCCUGCUCGACAUCCUC | TCLLDILDTAGHEEYSAM |
| | | GAUACCGCCGGUCACGAAGAG | RDQYMRTGEGFLC |
| | | UACUCCGCCAUGCGUGAUCAA | (SEQ ID NO: 372) |
| | | UAUAUGCGAACGGGGGAGGGU | |
| | | UUCUUAUGC (SEQ ID NO: 116) | |
| DTT(96-177) | 82 | AAUUUGGAGAAAACCACCGCA | NLEKTTAALSILPGIGSV |
| | | GCACUAUCUAUCCUGCCGGGA | MGIADGAVHHNTEEIVA |
| | | AUAGGUAGCGUCAUGGGCAUC | QSIALSSLMVAQAIPLVG |
| | | GCCGACGGAGCAGUACAUCAU | ELVDIGFAAYNFVESIINL |
| | | AAUACUGAAGAAAUUGUGGCC | FQVVHNSYNR (SEQ ID |
| | | CAAUCUAUCGCCUUAAGUUCU | NO: 373) |
| | | UUGAUUGGUGGCUCAGGCUAU | |
| | | UCCCCUGGUCGGGGAGCUGG | |
| | | UAGAUAUUGGCUUCGCUGCCU | |
| | | ACAACUUCGUGGAGAGCAUCA | |
| | | UUAAUUUGUUCCAGGUCGUGC | |
| | | AUAAUAGCUACAACAGG (SEQ | |
| | | ID NO: 117) | |

Antigen 11 is a peptide in which a DTT domain (1-87aa), six variant peptides, and a DTT domain (96-177aa) are sequentially linked with a linker as described in Table 12. Table 13 shows the variant peptides included in Antigen 12 and their mRNA sequences.

TABLE 13

| Peptide | Length | | |
|---|---|---|---|
| name | (aa) | mRNA | Peptide |
| 8-G12C-8 | 17 | UACAAGCUAGUGGUGGUCGGCGCA | YKLVVVGACGVGKS |
| | | UGCGGCGUGGGAAAGAGCGCGCUA | ALT (SEQ ID NO: |
| | | ACC (SEQ ID NO: 118) | 374) |
| 8-G12D-8 | 17 | UAUAAAUUGGUCGUUGUGGGCGCC | YKLVVVGADGVGKS |
| | | GAUGGUGUAGGGAAAUCUGCGCUG | ALT (SEQ ID NO: |
| | | ACC(SEQ ID NO: 119) | 375) |
| 8-G12V-8 | 17 | UACAAGCUCGUGGUGGUGGGGGCA | YKLVVVGAVGVGKS |
| | | GUCGGCGUCGGCAAAAGUGCCCUG | ALT (SEQ ID NO: |
| | | ACU (SEQ ID NO: 120) | 376) |
| 8-G12R-8 | 17 | UAUAAGCUGGUCGUGGUAGGUGCU | YKLVVVGARGVGKS |
| | | CGUGGCGUUGGAAAGUCCGCCUUG | ALT (SEQ ID NO: |
| | | ACA (SEQ ID NO: 121) | 377) |
| 8-G13C-8 | 17 | AAGCUGGUUGUCGUGGGGGCCGGA | KLVVVGAGCVGKSA |
| | | UGCGUGGGCAAAUCUGCUCUUACAA | LTI (SEQ ID NO: |
| | | UC (SEQ ID NO: 122) | 378) |
| 8-G13D-8 | 17 | AAAUUAGUAGUUGUGGGAGCUGGA | KLVVVGAGDVGKSA |
| | | GACGUAGGUAAAUCCGCACUGACGA | LTI (SEQ ID NO: |
| | | UA (SEQ ID NO: 123) | 379) |
| 8-Q61H-8 | 17 | CUCGAUAUUCUCGACACCGCUGGAC | LDILDTAGHEEYSA |
| | | ACGAAGAGUACAGCGCCAUGCGCGA | MRD (SEQ ID NO: |
| | | C (SEQ ID NO: 124) | 380) |
| 8-G13R-8 | 17 | AAGCUGGUCGUGGUGGGGGCCGGC | KLVVVGAGRVGKSA |
| | | AGAGUUGGUAAGUCAGCACUCACUA | LTI (SEQ ID NO: |
| | | UC (SEQ ID NO: 125) | 381) |

Table 14 shows the variant peptides contained in Antigen 13 and their mRNA sequences.

TABLE 14

| Peptide | Length | | |
|---|---|---|---|
| name | (aa) | mRNA | Peptide |
| 11-G12C-19 | 31 | AUGACCGAGUACAAACUCGUUGUGG | MTEYKLVVVGACGV |
| | | UGGGGGCCUGCGGAGUGGGCAAGU | GKSALTIQLIQNHFV |
| | | CUGCAUUGACCAUACAGUUGAUCCA | DE(SEQ ID NO: 382) |
| | | GAAUCAUUUCGUGGAUGAG (SEQ ID | |
| | | NO: 126) | |
| 3-G12C-4 | 8 | GUCGGAGCGUGUGGCGUGGGCAAA | VGACGVGK (SEQ ID |
| | | (SEQ ID NO: 127) | NO: 383) |
| 11-G12D-19 | 31 | AUGACUGAGUAUAAGUUAGUAGUCG | MTEYKLVVVGADGV |
| | | UUGGUGCCGAUGGAGUAGGCAAAU | GKSALTIQLIQNHFVDE |
| | | CGGCGUUAACGAUCCAGCUUAUUCA | (SEQ ID NO: 384) |
| | | GAAUCAUUUCGUAGAUGAG (SEQ ID | |
| | | NO: 128) | |
| 3-G12D-4 | 8 | GUCGGGGCCGACGGCGUGGGCAAA | VGADGVGK (SEQ ID |
| | | (SEQ ID NO: 129) | NO: 385) |
| 11-G12V-19 | 31 | AUGACCGAGUAUAAGUUAGUCGUCG | MTEYKLVVVGAVGV |
| | | UGGGUGCUGUGGGCGUAGGUAAAU | GKSALTIQLIQNHFV |
| | | CCGCCCUGACUAUACAGCUUAUUCA | DE (SEQ ID NO: 386) |
| | | AAACCAUUUUGUGGAUGAG (SEQ ID | |
| | | NO: 130) | |
| 3-G12V-4 | 8 | GUGGGAGCUGUGGGCGUUGGGAAA | VGAVGVGK (SEQ ID |
| | | (SEQ ID NO: 131) | NO: 387) |
| 11-G12R-19 | 31 | AUGACUGAGUACAAGCUGGUGGUC | MTEYKLVVVGARGV |
| | | GUCGGUGCCCGGGGCGUUGGUAAG | GKSALTIQLIQNHFV |
| | | AGCGCCCUGACCAUACAACUCAUUC | DE (SEQ ID NO: 388) |
| | | AGAACCACUUUGUAGACGAA (SEQ | |
| | | ID NO: 132) | |
| 3-G12R-4 | 8 | GUGGGAGCAAGGGGAGUCGGGAAA | VGARGVGK (SEQ ID |
| | | (SEQ ID NO: 133) | NO: 389) |
| 11-G13C-19 | 31 | ACAGAAUACAAGCUGGUUGUAGUCG | TEYKLVVVGAGCVG |
| | | GCGCCGGUUGUGUCGGGAAGUCCG | KSALTIQLIQNHFVD |
| | | CACUCACAAUUCAGCUUAUCCAGAA | EY (SEQ ID NO: 390) |
| | | UCACUUCGUGGACGAGUAU (SEQ ID | |
| | | NO: 134) | |
| 3-G13C-4 | 8 | GGGGCAGGCUGCGUUGGAAAGAGC | GAGCVGKS (SEQ ID |
| | | (SEQ ID NO: 135) | NO: 391) |
| 11-G13D-19 | 31 | ACAGAGUACAAAUUAGUGGUCGUGG | TEYKLVVVGAGDVG |
| | | GAGCUGGGGACGUGGGAAAGUCCG | KSALTIQLIQNHFVD |
| | | CUCUCACUAUCCAGCUGAUUCAGAA | EY (SEQ ID NO: 392) |
| | | CCAUUUUGUCGACGAAUAU (SEQ ID | |
| | | NO: 136) | |
| 3-G13D-4 | 8 | GGGGCUGGGGACGUGGGCAAAUCA | GAGDVGKS (SEQ ID |
| | | (SEQ ID NO: 137) | NO: 393) |
| 11-Q61H-19 | 31 | ACCUGCCUGCUGGAUAUCCUGGACA | TCLLDILDTAGHEEY |
| | | CAGCGGGACACGAAGAAUACAGCGC | SAMRDQYMRTGEG |
| | | CAUGAGAGAUCAGUACAUGCGAACG | FLC (SEQ ID NO: |
| | | GGUGAAGGCUUCCUGUGC (SEQ ID | 394) |
| | | NO: 138) | |
| 3-Q61H-4 | 8 | ACAGCAGGGCACGAGGAAUACUCC | TAGHEEYS (SEQ ID |
| | | (SEQ ID NO: 139) | NO: 395) |
| 11-G13R-19 | 31 | ACUGAAUAUAAGCUGGUAGUUGUUG | TEYKLVVVGAGRVG |
| | | GCGCAGGAAGAGUUGGGAAGAGUG | KSALTIQLIQNHFVD |
| | | CUCUAACCAUCCAACUUAUCCAGAA | EY (SEQ ID NO: 396) |
| | | CCACUUUGUGGACGAGUAU (SEQ ID | |
| | | NO: 140) | |
| 3-G13R-4 | 8 | GGCGCCGGGCGCGUCGGAAAGUCU | GAGRVGKS (SEQ ID |
| | | (SEQ ID NO: 141) | NO: 397) |

Table 15 shows the variant peptides contained in Antigen 14 and their mRNA sequences.

TABLE 15

| Peptide | Length | | |
|---|---|---|---|
| name | (aa) | mRNA | Peptide |
| 11-G12C-19 | 31 | AUGACAGAAUACAAGUUAGUGGUGG | MTEYKLVVVGACGV |
| | | UAGGAGCGUGUGGAGUGGGGAAAA | GKSALTIQLIQNHFV |
| | | GCGCCCUAACUAUUCAGUUGAUACA | DE (SEQ ID NO: 398) |
| | | AACCAUUUCGUGGACGAA (SEQ ID | |
| | | NO: 142) | |
| 3-G12C-4 | 8 | GUGGGCGCAUGUGGAGUCGGCAAG | VGACGVGK (SEQ ID |
| | | (SEQ ID NO: 143) | NO: 399) |
| 11-G12D-19 | 31 | AUGACCGAGUAUAAGCUCGUAGUCG | MTEYKLVVVGADGV |
| | | UGGGGGCCGACGGCGUGGGGAAAU | GKSALTIQLIQNHFV |
| | | CAGCCCUUACUAUCCAGCUUAUACA | DE (SEQ ID NO: 400) |
| | | AAACCAUUUUGUUGACGAA | |
| | | (SEQ ID NO: 144) | |
| 3-G12D-4 | 8 | GUAGGCGCCGACGGGGUCGGAAAG | VGADGVGK (SEQ ID |
| | | (SEQ ID NO: 145) | NO: 401) |
| 11-G12V-19 | 31 | AUGACCGAGUACAAGCUGGUUGUC | MTEYKLVVVGAVGV |
| | | GUCGGGGGCCGUAGGGGUUGGCAA | GKSALTIQLIQNHFV |
| | | AAGCGCCCUGACUAUCCAGCUCAUC | DE (SEQ ID NO: 402) |
| | | CAAAAUCAUUUCGUCGACGAG (SEQ | |
| | | ID NO: 146) | |
| 3-G12V-4 | 8 | GUGGGCGCCGUUGGAGUCGGUAAA | VGAVGVGK (SEQ ID |
| | | (SEQ ID NO: 147) | NO: 403) |
| 11-G12R-19 | 31 | AUGACGGAAUACAAGUUGGUGGUAG | MTEYKLVVVGARGV |
| | | UGGGGGCUCGGGGGGUGGGCAAGA | GKSALTIQLIQNHFV |
| | | GUGCUUUGACCAUACAGUUAAUUCA | DE (SEQ ID NO: 404) |
| | | GAACCACUUUGUGGAUGAG (SEQ ID | |
| | | NO: 148) | |
| 3-G12R-4 | 8 | GUCGGCGCUCGCGGAGUCGGCAAA | VGARGVGK (SEQ ID |
| | | (SEQ ID NO: 149) | NO: 405) |
| 11-G13C-19 | 31 | ACCGAGUAUAAAUUGGUUGUUGUCG | TEYKLVVVGAGCVG |
| | | GCGCAGGCUGUGUGGGCAAGUCUG | KSALTIQLIQNHFVD |
| | | CUCUCACCAUUCAACUAAUCCAGAA | EY (SEQ ID NO: 406) |
| | | CCACUUUGUUGAUGAGUAC (SEQ ID | |
| | | NO: 150) | |
| 3-G13C-4 | 8 | GGAGCAGGCUGCGUGGGAAAAAGC | GAGCVGKS (SEQ ID |
| | | (SEQ ID NO: 151) | NO: 407) |
| 11-G13D-19 | 31 | ACGGAGUACAAGCUGGUUGUCGUA | TEYKLVVVGAGDVG |
| | | GGAGCAGGAGACGUGGGCAAAUCC | KSALTIQLIQNHFVD |
| | | GCUCUGACAAUUCAGCUUAUCCAGA | EY (SEQ ID NO: 408) |
| | | AUCACUUUGUGGAUGAAUAU (SEQ | |
| | | ID NO: 152) | |
| 3-G13D-4 | 8 | GGUGCCGGGGAUGUCGGUAAAAGU | GAGDVGKS (SEQ ID |
| | | (SEQ ID NO: 153) | NO: 409) |
| 11-Q61H-19 | 31 | ACCUGCUUACUGGACAUCCUGGACA | TCLLDILDTAGHEEY |
| | | CUGCUGGUCACGAGGAAUACUCCG | FLC (SEQ ID NO: |
| | | CGAUGCGAGAUCAGUAUAUGCGGAC | 410) |
| | | AGGAGAGGGUUUCCUGUGC (SEQ ID | SAMRDQYMRTGEG |
| | | NO: 154) | |
| 3-Q61H-4 | 8 | ACAGCAGGCCAUGAGGAAUAUUCU | TAGHEEYS (SEQ ID |
| | | (SEQ ID NO: 155) | NO: 411) |
| 11-G13R-19 | 31 | ACUGAAUAUAAGCUGGUGGUGGUG | TEYKLVVVGAGRVG |
| | | GGGGCUGGUAGAGUGGGGAAGUCA | KSALTIQLIQNHFVD |
| | | GCACUGACAAUCCAGCUCAUUCAGA | EY (SEQ ID NO: 412) |
| | | AUCACUUCGUUGAUGAGUAC (SEQ | |
| | | ID NO: 156) | |
| 3-G13R-4 | 8 | GGUGCGGGUAGAGUGGGCAAGUCC | GAGRVGKS (SEQ ID |
| | | (SEQ ID NO: 157) | NO: 413) |

Table 16 shows the variant peptides contained in Antigen 15 and their mRNA sequences.

TABLE 16

| Peptide | Length | | |
|---|---|---|---|
| name | (aa) | mRNA | Peptide |
| 11-G12D-19 | 31 | AUGACGGAAUACAAGCUCGUCGUGG | MTEYKLVVVGADGV |
| | | UGGGCGCCGAUGGCGUCGGCAAGU | GKSALTIQLIQNHFV |
| | | CGGCACUGACUAUCCAGCUCAUACA | DE (SEQ ID NO: 414) |
| | | AAAUCAUUUUGUUGAUGAA | |
| | | (SEQ ID NO: 158) | |
| 3-G12D-4 | 8 | GUGGGGGCGGACGGAGUGGGGAAG | VGADGVGK (SEQ ID |
| | | (SEQ ID NO: 159) | NO: 415) |
| 11-G12C-19 | 31 | AUGACCGAAUACAAACUGGUGGUGG | MTEYKLVVVGACGV |
| | | UCGGGGCGUGUGGGGGGGGCAAAU | GKSALTIQLIQNHFV |
| | | CAGCCCUCACAAUACAGCUGAUUCA | DE (SEQ ID NO: 416) |
| | | GAACCACUUUGUUGACGAG | |
| | | (SEQ ID NO: 160) | |
| 3-G12C-4 | 8 | GUGGGGGCAUGUGGAGUGGGGAAG | VGACGVGK (SEQ ID |
| | | (SEQ ID NO: 161) | NO: 417) |
| 11-G12V-19 | 31 | AUGACUGAGUACAAGCUGGUUGUU | MTEYKLVVVGAVGV |
| | | GUAGGUGCUGUUGGAGUGGGAAAG | GKSALTIQLIQNHFV |
| | | AGCGCCCUCACAAUCCAAUUGAUCC | DE (SEQ ID NO: 418) |
| | | AGAAUCACUUCGUCGACGAG (SEQ | |
| | | ID NO: 162) | |
| 3-G12V-4 | 8 | GUGGGGGCCGUAGGCGUGGGCAAG | VGAVGVGK (SEQ ID |
| | | (SEQ ID NO: 163) | NO: 419) |
| 11-G12R-19 | 31 | AUGACCGAAUAUAAGUUGGUAGUGG | MTEYKLVVVGARGV |
| | | UGGGGGCUCGCGGAGUCGGGAAGA | DE (SEQ ID NO: 420) |
| | | GUGCUCUUACUAUUCAGCUGAAUUC | GKSALTIQLIQNHFV |
| | | AGAACCAUUUCGUAGAUGAA (SEQ | |
| | | ID NO: 164) | |
| 3-G12R-4 | 8 | GUGGGCGCCAGAGGCGUGGGCAAA | VGARGVGK (SEQ ID |
| | | (SEQ ID NO: 165) | NO: 421) |
| 11-G13C-19 | 31 | ACUGAGUACAAACUAGUAGUCGUAG | TEYKLVVVGAGCVG |
| | | GAGCUGGGUGUGUGGGCAAAAGUG | KSALTIQLIQNHFVD |
| | | CACUCACCAUCCAGCUGAUCCAGAA | EY (SEQ ID NO: 422) |
| | | CCAUUUCGUGGAUGAGUAU (SEQ ID | |
| | | NO: 166) | |
| 3-G13C-4 | 8 | GGAGCUGGCUGCGUGGGCAAAAGC | GAGCVGKS (SEQ ID |
| | | (SEQ ID NO: 167) | NO: 423) |
| 11-G13D-19 | 31 | ACGGAAUACAAACUAGUUGUUGUGG | TEYKLVVVGAGDVG |
| | | GGGCUGGUGAUGUCGGAAAGUCCG | KSALTIQLIQNHFVD |
| | | CCUUGACUAUUCAGCUUAUUCAAAA | EY (SEQ ID NO: 424) |
| | | UCACUUUGUCGACGAGUAU (SEQ ID | |
| | | NO: 168) | |
| 3-G13D-4 | 8 | GGCGCAGGUGACGUAGGAAAGUCU | GAGDVGKS (SEQ ID |
| | | (SEQ ID NO: 169) | NO: 425) |
| 11-Q61H-19 | 31 | ACCUGCCUUCUGGACAUCCUGGACA | TCLLDILDTAGHEEY |
| | | CAGCCGGCCACGAGGAAUACAGCGC | SAMRDQYMRTGEG |
| | | CAUGCGGGAUCAGUAUAUGAGGACA | FLC (SEQ ID NO: |
| | | GGAGAAGGCUUCCUCUGC (SEQ ID | 426) |
| | | NO: 170) | |
| 3-Q61H-4 | 8 | ACAGCUGGCCACGAGGAGUAUUCU | TAGHEEYS (SEQ ID |
| | | (SEQ ID NO: 171) | NO: 427) |
| 11-G13R-19 | 31 | ACCGAGUACAAACUUGUGGGUGGU | TEYKLVVVGAGRVG |
| | | UGGAGCAGGGCGUGUCGGCAAAUC | KSALTIQLIQNHFVD |
| | | UGCCCUGACCAUCCAGCUGAUACAA | EY (SEQ ID NO: 428) |
| | | AACCAUUUUGUGGAUGAGUAU (SEQ | |
| | | ID NO: 172) | |
| 3-G13R-4 | 8 | GGCGGUGCGGGACGAGUGGGCAAG | GAGRVGKS (SEQ ID |
| | | AGC (SEQ ID NO: 173) | NO: 429) |

Table 17 shows the variant peptides contained in Antigen 16 and their mRNA sequences.

TABLE 17

| | Length | | |
|---|---|---|---|
| Peptide name | (aa) | mRNA | Peptide |
| 8-G12C-8 | 17 | UAUAAGUUGGUGGUGGUUGGGGCA | YKLVVVGACGVGKS |
| | | UGUGGCGUAGGUAAGAGCGCACUG | ALT (SEQ ID NO: |
| | | ACU (SEQ ID NO: 174) | 430) |
| 8-G12D-8 | 17 | UAUAAGCUAGUUGUCGUGGGAGCU | YKLVVVGADGVGKS |
| | | GACGGAGUGGGGAAGAGUGCACUC | ALT (SEQ ID NO: |
| | | ACA (SEQ ID NO: 175) | 431) |
| 8-G12V-8 | 17 | UACAAGCUCGUCGUGGUGGGCCGC | YKLVVVGAVGVGKS |
| | | CGUCGGCGUAGGGAAAUCAGCUCU | ALT (SEQ ID NO: |
| | | UACC (SEQ ID NO: 176) | 432) |
| 8-G12R-8 | 17 | UAUAAACUCGUGGUGGUGGGCGCU | YKLVVVGARGVGKS |
| | | CGCGGUGUGGGCAAAAGUGCCCUG | ALT (SEQ ID NO: |
| | | ACC (SEQ ID NO: 177) | 433) |
| 8-G13C-8 | 17 | AAGCUGGUCGUCGUGGGCGCCGGC | KLVVVGAGCVGKSA |
| | | UGCGUUGGGAAAUCCGCCCUGACAA | LTI (SEQ ID NO: |
| | | UC (SEQ ID NO: 178) | 434) |
| 8-G13D-8 | 17 | AAAUUGGUCGUCGUGGGCGCCGGG | KLVVVGAGDVGKSA |
| | | GACGUGGGUAAGUCAGCCUUGACC | LTI (SEQ ID NO: |
| | | AUC (SEQ ID NO: 179) | 435) |
| 8-Q61H-8 | 17 | CUCGACAUACUGGACACUGCCGGCC | LDILDTAGHEEYSA |
| | | ACGAAGAAUAUUCUGCCAUGAGGGA | MRD (SEQ ID NO: |
| | | U (SEQ ID NO: 180) | 436) |
| 8-G13R-8 | 17 | AAAUUAGUGGUGGUAGGUGCCGGU | KLVVVGAGRVGKSA |
| | | AGAGUGGGGAAGUCGGCUCUCACC | LTI (SEQ ID NO: |
| | | AUU (SEQ ID NO: 181) | 437) |
| 8-G12C-8 | 17 | UACAAGCUGGGUGGUAGUCGGCGC | YKLVVVGACGVGKS |
| | | AUGUGGAGUGGGAAAAAGCGCCCU | ALT (SEQ ID NO: |
| | | GACA (SEQ ID NO: 182) | 438) |
| 8-G12D-8 | 17 | UACAAAUUAGUGGUGGUCGGUGCU | YKLVVVGADGVGKS |
| | | GAUGGGGUCGGCAAGUCAGCUCUG | ALT (SEQ ID NO: |
| | | ACC (SEQ ID NO: 183) | 439) |
| 8-G12V-8 | 17 | UACAAACUCGUUGUCGUUGGGGCC | YKLVVVGAVGVGKS |
| | | GUCGGAGUGGGUAAAUCCGCCCUG | ALT (SEQ ID NO: |
| | | ACC (SEQ ID NO: 184) | 440) |
| 8-G12R-8 | 17 | UAUAAAUUGGUCGUGGUAGGGGCU | YKLVVVGARGVGKS |
| | | CGGGGAGUCGGGAAAUCUGCGCUG | ALT (SEQ ID NO: |
| | | ACC (SEQ ID NO: 185) | 441) |
| 8-G13C-8 | 17 | AAGCUCGUUGUGGUGGGAGCAGGU | KLVVVGAGCVGKSA |
| | | UGCGUUGGUAAGUCUGCUUUGACU | LTI (SEQ ID NO: |
| | | AUC (SEQ ID NO: 186) | 442) |
| 8-Q61H-8 | 17 | CUCGACAUCCUCGACACCGCAGGAC | LDILDTAGHEEYSA |
| | | AUGAGGAGUACUCCGCCAUGCGUG | MRD (SEQ ID NO: |
| | | AC (SEQ ID NO: 187) | 443) |
| 8-G13D-8 | 17 | AAGCUAGUAGUUGUAGGAGCCGGU | KLVVVGAGDVGKSA |
| | | GAUGUGGGAAAGUCCGCUCUGACAA | LTI (SEQ ID NO: |
| | | UU (SEQ ID NO: 188) | 444) |
| 8-G13R-8 | 17 | AAAUUGGUAGUCGUGGGGGCCGGC | KLVVVGAGRVGKSA |
| | | AGAGUGGGAAAAAGCGCGCUGACAA | LTI (SEQ ID NO: |
| | | UC (SEQ ID NO: 189) | 445) |
| 8-G12V-8 | 17 | UAUAAAACUUGUUGUGGUGGGCGC | YKLVVVGAVGVGKS |
| | | AGUCGGAGUUGGUAAGUCAGCUCU | ALT (SEQ ID NO: |
| | | GACU(SEQ ID NO: 190) | 446) |
| 8-G12R-8 | 17 | UACAAGCUCGUUGUUGUGGGGCGC | YKLVVVGARGVGKS |
| | | UCGCGGGGUGGGCAAGUCUGCCCU | ALT (SEQ ID NO: |
| | | AACA (SEQ ID NO: 191) | 447) |
| 8-G13C-8 | 17 | AAACUAGUGGUGGUCGGCGCCGGG | KLVVVGAGCVGKSA |
| | | UGCGUAGGAAAGAGUGCUCUGACCA | LTI (SEQ ID NO: |
| | | UA (SEQ ID NO: 192) | 448) |
| 8-Q61H-8 | 17 | CUUGACAUUCUGGAUACUGCGGGC | LDILDTAGHEEYSA |
| | | CACGAGGAAUAUAGCGCGAUGCGAG | MRD (SEQ ID NO: |
| | | AU (SEQ ID NO: 193) | 449) |
| 8-G12C-8 | 17 | UACAAACUGGUUGUUGUGGGAGCA | YKLVVVGACGVGKS |
| | | UGUGGCGUCGGGAAGUCCGCAUUA | ALT (SEQ ID NO: |
| | | ACG (SEQ ID NO: 194) | 450) |
| 8-G12D-8 | 17 | UACAAGUUAGUGGUGGUCGGAGCG | YKLVVVGADGVGKS |
| | | GAUGGGCGUUGGGAAAGUCUGCAC | ALT (SEQ ID NO: |
| | | UUACG (SEQ ID NO: 195) | 451) |
| 8-G13D-8 | 17 | AAGUUGGUGGUGGUGGGGGCCGGA | KLVVVGAGDVGKSA |
| | | GACGUAGGCAAGUCCGCACUUACCA | LTI (SEQ ID NO: |
| | | UC (SEQ ID NO: 196) | 452) |
| 8-G13R-8 | 17 | AAACUGGUUGUCGUCGGCGCCGGG | KLVVVGAGRVGKSA |
| | | CGGGUGGGCAAGAGCGCACUGACU | LTI (SEQ ID NO: |
| | | AUU (SEQ ID NO: 197) | 453) |

Table 18 shows the variant peptides contained in Antigen 17 and their mRNA sequences.

TABLE 18

| | Length | | |
|---|---|---|---|
| Peptide name | (aa) | mRNA | Peptide |
| 11-G12D-19 | 31 | AUGACGGAAUACAAGCUCGUCGUGG | MTEYKLVVVGADGV |
| | | UGGGCGCCGAUGGCGUCGGCAAGU | GKSALTIQLIQNHFV |
| | | CGGCACUGACUAUCCAGCUCAUACA | DE (SEQ ID NO: 454) |
| | | AAAUCAUUUUGUUGAUGAA (SEQ ID | |
| | | NO: 198) | |
| 3-G12D-4 | 8 | GUGGGGGCGGACGGAGUGGGGAAG | VGADGVGK (SEQ ID |
| | | (SEQ ID NO: 199) | NO: 455) |
| 11-G12C-19 | 31 | AUGACCGAAUACAAACUGGUGGUGG | MTEYKLVVVGACGV |
| | | UCGGGGGCUGUGGGGGGUGGGCAA | GKSALTIQLIQNHFV |
| | | AUCAGCCCUCACAAUACAGCUGAUU | DE (SEQ ID NO: 456) |
| | | CAGAACCACUUUGUUGACGAG (SEQ | |
| | | ID NO: 200) | |
| 3-G12C-4 | 8 | GUGGGGGCAUGUGGAGUGGGGAAG | VGACGVGK (SEQ ID |
| | | (SEQ ID NO: 201) | NO: 457) |
| 11-G12V-19 | 31 | AUGACUGAGUACAAGCUGGUUGUU | MTEYKLVVVGAVGV |
| | | GUAGGUGCUGUUGGAGUGGGAAAG | GKSALTIQLIQNHFV |
| | | AGCGCCCUCACAAUCCAAUUGAUCC | DE (SEQ ID NO: 458) |
| | | AGAAUCACUUCGUCGACGAG (SEQ | |
| | | ID NO: 202) | |
| 3-G12V-4 | 8 | GUGGGGGCCGUAGGCGUGGGCAAG | VGAVGVGK (SEQ ID |
| | | (SEQ ID NO: 203) | NO: 459) |
| 11-G12R-19 | 31 | AUGACCGAAUAUAAGUUGGUAGUGG | MTEYKLVVVGARGV |
| | | UGGGGGCUCGCGGAGUCGGGAAGA | GKSALTIQLIQNHFV |
| | | GUGCUCUUACUAUUCAGCUGAAUUC | DE (SEQ ID NO: 460) |
| | | AGAACCAUUUCGUAGAUGAA (SEQ | |
| | | ID NO: 204) | |
| 3-G12R-4 | 8 | GUGGGCGCCAGAGGCGUGGGCAAA | VGARGVGK (SEQ ID |
| | | (SEQ ID NO: 205) | NO: 461) |
| 11-G13C-19 | 31 | ACUGAGUACAAACUAGUAGUCGUAG | TEYKLVVVGAGCVG |
| | | GAGCUGGGUGUGUGGGCAAAAGUG | KSALTIQLIQNHFVD |
| | | CACUCACCAUCCAGCUGAUCCAGAA | EY (SEQ ID NO: 462) |
| | | CCAUUUCGUGGAUGAGUAU (SEQ ID | |
| | | NO: 206) | |
| 3-G13C-4 | 8 | GGAGCUGGCUGCGUGGGCAAAAGC | GAGCVGKS (SEQ ID |
| | | (SEQ ID NO: 207) | NO: 463) |
| 11-G13D-19 | 31 | ACGGAAUACAAACUAGUUGUUGUGG | TEYKLVVVGAGDVG |
| | | GGGCUGGUGAUGUCGGAAAGUCCG | KSALTIQLIQNHFVD |
| | | CCUUGACUAUUCAGCUUAUUCAAAA | EY (SEQ ID NO: 464) |
| | | UCACUUUGUCGACGAGUAU (SEQ ID | |
| | | NO: 208) | |
| 3-G13D-4 | 8 | GGCGCAGGUGACGUAGGAAAGUCU | GAGDVGKS (SEQ ID |
| | | (SEQ ID NO: 209) | NO: 465) |
| 11-Q61H-19 | 31 | ACCUGCCUUCUGGACAUCCUGGACA | TCLLDILDTAGHEEY |
| | | CAGCCGGCCACGAGGAAUACAGCGC | SAMRDQYMRTGEG |
| | | CAUGCGGGAUCAGUAUAUGAGGACA | FLC (SEQ ID NO: |
| | | GGAGAAGGCUUCCUCUGC (SEQ ID | 466) |
| | | NO: 210) | |
| 3-Q61H-4 | 8 | ACAGCUGGCCACGAGGAGUAUUCU | TAGHEEYS (SEQ ID |
| | | (SEQ ID NO: 211) | NO: 467) |

Table 19 shows the variant peptides contained in Antigen 18 and their mRNA sequences.

TABLE 19

| | Length | | |
|---|---|---|---|
| Peptide name | (aa) | mRNA | Peptide |
| 11-G12D-19 | 31 | AUGACAGAGUAUAAACUCGUGGUGG | MTEYKLVVVGADGV |
| | | UCGGCGCAGAUGGCGUUGGAAAGA | GKSALTIQLIQNHFV |
| | | GCGCCCUGACUAUUCAGCUCAUCCA | DE(SEQ ID NO: 468) |
| | | GAACCAUUUCGUAGACGAA (SEQ ID | |
| | | NO: 212) | |
| 3-G12D-4 | 8 | GUCGGCGCAGACGGUGUGGGAAAG | VGADGVGK (SEQ ID |
| | | (SEQ ID NO: 213) | NO: 469) |
| 11-G12C-19 | 31 | AUGACCGAAUACAAGUUAGUGGUGG | MTEYKLVVVGACGV |
| | | UCGGGGCCUGCGGGUGUGGGUAAA | GKSALTIQLIQNHFV |
| | | AGCGCUCUGACCAUACAGCUUAUCC | DE (SEQ ID NO: 470) |
| | | AGAACCACUUCGUGGAUGAG (SEQ | |
| | | ID NO: 214) | |
| 3-G12C-4 | 8 | GUGGGGGCUUGCGGAGUUGGCAAG | VGACGVGK (SEQ ID |
| | | (SEQ ID NO: 215) | NO: 471) |
| 11-G12V-19 | 31 | AUGACAGAAUAUAAAUUGGUGGUAG | MTEYKLVVVGAVGV |
| | | UGGGGGCCGUAGGCGUCGGCAAGA | GKSALTIQLIQNHFV |
| | | GCGCCCUGACUAUACAACUGAUCCA | DE (SEQ ID NO: 472) |
| | | AAACCACUUCGUGGACGAG (SEQ ID | |
| | | NO: 216) | |
| 3-G12V-4 | 8 | GUGGGCGCCGUUGGAGUCGGGAAA | VGAVGVGK (SEQ ID |
| | | (SEQ ID NO: 217) | NO: 473) |
| 11-G12R-19 | 31 | AUGACUGAGUAUAAACUUGUUGUAG | MTEYKLVVVGARGV |
| | | UCGGGGCACGGGGCGUGGGAAAGU | GKSALTIQLIQNHFV |
| | | CCGCUUUAACCAUACAAUUGAUACA | DE (SEQ ID NO: 474) |
| | | GAACCACUUUGUUGAUGAA (SEQ ID | |
| | | NO: 218) | |
| 3-G12R-4 | 8 | GUGGGUGCCAGGGGAGUCGGUAAG | VGARGVGK (SEQ ID |
| | | (SEQ ID NO: 219) | NO: 475) |
| 11-Q61H-19 | 31 | ACCUGUCUCCUAGACAUUCUGGACA | TCLLDILDTAGHEEY |
| | | CAGCCGGCCACGAGGAGUACUCAG | SAMRDQYMRTGEG |
| | | CUAUGCGCGAUCAGUAUAUGAGAAC | FLC (SEQ ID NO: |
| | | AGGGGAGGGAUUUCUGUGU (SEQ ID | 476) |
| | | NO: 220) | |
| 3-Q61H-4 | 8 | ACAGCAGGGCACGAAGAGUACAGC | TAGHEEYS (SEQ ID |
| | | (SEQ ID NO: 221) | NO: 477) |
| 11-G13D-19 | 31 | ACUGAGUACAAGCUAGUCGUGGUU | TEYKLVVVGAGDVG |
| | | GGCGCAGGCGACGUGGGUAAAUCU | KSALTIQLIQNHFVD |
| | | GCCCUCACCAUUCAGUUAAUUCAGA | EY (SEQ ID NO: 478) |
| | | AUCAUUUCGUCGAUGAAUAU (SEQ | |
| | | ID NO: 222) | |
| 3-G13D-4 | 8 | GGCGCUGGAGACGUCGGCAAAUCC | GAGDVGKS (SEQ ID |
| | | (SEQ ID NO: 223) | NO: 479) |
| 11-G12A-19 | 31 | AUGACGGAGUAUAAACUGGUAGUCG | MTEYKLVVVGAAGV |
| | | UAGGUGCUGCCGGCGUCGGCAAAA | GKSALTIQLIQNHFV |
| | | GCGCGCUAACCAUCCAGCUGAUCCA | DE (SEQ ID NO: 480) |
| | | GAAUCACUUUGUGGAUGAA (SEQ ID | |
| | | NO: 224) | |
| 3-G12A-4 | 8 | GUGGGAGCCGCUGGAGUGGGGAAG | VGAAGVGK (SEQ ID |
| | | (SEQ ID NO: 225) | NO: 481) |
| 11-A146T-19 | 31 | AGAUCUUACGGAAUCCCUUUCAUCG | RSYGIPFIETSTKTR |
| | | AAACCUCAACCAAGACUCGUCAAAG | QRVEDAFYTLVREIR |
| | | GGUUGAAGACGCUUUCUAUACACUU | Q (SEQ ID NO: 482) |
| | | GUCAGAGAGAUUAGGCAG (SEQ ID | |
| | | NO: 226) | |
| 3-A146T-4 | 8 | GAAACCUCCACAAAGACGCGGCAA | ETSTKTRQ (SEQ ID |
| | | (SEQ ID NO: 227) | NO: 483) |
| 11-G13C-19 | 31 | ACUGAGUACAAGCUGGUGGUAGUG | TEYKLVVVGAGCVG |
| | | GGAGCCGGUUGCGUGGGCAAGUCA | KSALTIQLIQNHFVD |
| | | GCGCUGACAAUCCAGCUCAUCCAGA | EY (SEQ ID NO: 484) |
| | | AUCAUUUUGUUGAUGAGUAC (SEQ | |
| | | ID NO: 228) | |
| 3-G13C-4 | 8 | GGCGCAGGCUGCGUGGGGAAAUCG | GAGCVGKS (SEQ ID |
| | | (SEQ ID NO: 229) | NO: 485) |
| 11-G12S-19 | 31 | AUGACUGAGUACAAGCUGGUGGUG | MTEYKLVVVGASGV |
| | | GUGGGGGCGUCUGGGGUGGGGGAA | GKSALTIQLIQNHFV |
| | | AUCUGCACUGACGAUUCAACUGAUC | DE (SEQ ID NO: 486) |
| | | CAGAAUCAUUUUGUAGACGAG (SEQ | |
| | | ID NO: 230) | |
| 3-G12S-4 | 8 | GUGGGCGCGUCCGGCGUGGGUAAG | VGASGVGK (SEQ ID |
| | | (SEQ ID NO: 231) | NO: 487) |
| 11-Q61R-19 | 31 | ACCUGUUUGCUGGAUAUUCUGGACA | TCLLDILDTAGREEY |
| | | CUGCCGGACGAGAAGAAUACAGUGC | SAMRDQYMRTGEG |
| | | AAUGAGGGAUCAGUAUAUGCGAACC | FLC (SEQ ID NO: |
| | | GGAGAGGGCUUUCUUUGU (SEQ ID | 488) |
| | | NO: 232) | |
| 3-Q61R-4 | 8 | ACAGCCGGCCGCGAAGAGUACAGC | TAGREEYS (SEQ ID |
| | | (SEQ ID NO: 233) | NO: 489) |

Table 20 shows the variant peptides contained in Antigen 19 and their mRNA sequences.

TABLE 20

| | Length | | |
|---|---|---|---|
| Peptide name | (aa) | mRNA | Peptide |
| 11-G12C-19 | 31 | AUGACGGAAUAUAAGCUCGUGGUCG | MTEYKLVVVGACGV |
| | | UUGGGGCCUGUGGCGUGGGUAAGA | GKSALTIQLIQNHFV |
| | | GCGCACUAACCAUUCAGUUAAUCCA | DE (SEQ ID NO: 490) |
| | | GAACCACUUUGUUGAUGAA (SEQ ID | |
| | | NO: 234) | |
| 3-G12C-4 | 8 | GUUGGGGCCUGCGGCGUUGGAAAA | VGACGVGK (SEQ ID |
| | | (SEQ ID NO: 235) | NO: 491) |
| 11-G12V-19 | 31 | AUGACAGAGUACAAACUGGUCGUGG | MTEYKLVVVGAVGV |
| | | UGGGGGCUGUGGGUGUGGGCAAGU | GKSALTIQLIQNHFV |
| | | CCGCCCUGACUAUCCAACUGAUACA | DE (SEQ ID NO: 492) |
| | | GAACCAUUUCGUAGAUGAA (SEQ ID | |
| | | NO: 236) | |
| 3-G12V-4 | 8 | GUCGGCGCAGUCGGGGUAGGUAAA | VGAVGVGK (SEQ ID |
| | | (SEQ ID NO: 237) | NO: 493) |
| 11-G12D-19 | 31 | AUGACUGAGUAUAAACUCGUGGUUG | MTEYKLVVVGADGV |
| | | UGGGUGCAGAUGGAGUAGGAAAGA | GKSALTIQLIQNHFV |
| | | GUGCCCUCACAAUACAACUGAUUCA | DE (SEQ ID NO: 494) |
| | | GAAUCAUUUUGUCGAUGAA (SEQ ID | |
| | | NO: 238) | |
| 3-G12D-4 | 8 | GUGGGAGCUGAUGGUGUUGGCAAG | VGADGVGK (SEQ ID |
| | | (SEQ ID NO: 239) | NO: 495) |
| 11-Q61H-19 | 31 | ACCUGCCUGCUGGACAUCCUGGACA | TCLLDILDTAGHEEY |
| | | CCGCGGGGCACGAAGAGUAUAGCG | SAMRDQYMRTGEG |
| | | CUAUGCGGGACCAGUACAUGAGAAC | FLC (SEQ ID NO: |
| | | CGGUGAGGGCUUUCUGUGU (SEQ ID | 496) |
| | | NO: 240) | |
| 3-Q61H-4 | 8 | ACAGCCGGCCACGAGGAGUAUUCU | TAGHEEYS (SEQ ID |
| | | (SEQ ID NO: 241) | NO: 497) |
| 11-G13C-19 | 31 | ACUGAGUACAAGCUGGUGGUGGUU | TEYKLVVVGAGCVG |
| | | GGCGCUGGCUGUGUGGGGAAAUCU | KSALTIQLIQNHFVD |
| | | GCAUUAACGAUUCAACUGAUCCAGA | EY (SEQ ID NO: 498) |
| | | AUCACUUCGUCGAUGAGUAC (SEQ | |
| | | ID NO: 242) | |
| 3-G13C-4 | 8 | GGCGCAGGCUGCGUGGGCAAGUCA | GAGCVGKS (SEQ ID |
| | | (SEQ ID NO: 243) | NO: 499) |
| 11-G13D-19 | 31 | ACAGAGUACAAGCUGGUUGUCGUU | TEYKLVVVGAGDVG |
| | | GGGGCAGGGGACGUCGGGAAGUCC | KSALTIQLIQNHFVD |
| | | GCCCUGACUAUCCAGCUGAUUCAGA | EY (SEQ ID NO: 500) |
| | | ACCAUUUUGUGGACGAAUAU (SEQ | |
| | | ID NO: 244) | |
| 3-G13D-4 | 8 | GGUGCUGGAGACGUGGGAAAAUCC | GAGDVGKS (SEQ ID |
| | | (SEQ ID NO: 245) | NO: 501) |
| 11-G12A-19 | 31 | AUGACAGAGUACAAACUUGUGGUGG | MTEYKLVVVGAAGV |
| | | UUGGCGCCUGCUGGAGUAGGAAAA | GKSALTIQLIQNHFV |
| | | UCCGCCCUGACCAUACAACUUAUUC | DE (SEQ ID NO: 502) |
| | | AGAAUCACUUCGUGGACGAG (SEQ | |
| | | ID NO: 246) | |
| 3-G12A-4 | 8 | GUCGGGGCGGCCGGGGUGGGGAAG | VGAAGVGK (SEQ ID |
| | | (SEQ ID NO: 247) | NO: 503) |
| 11-G12S-19 | 31 | AUGACUGAAUACAAGUUGGUGGUG | MTEYKLVVVGASGV |
| | | GUGGGCGCGAGCGGGGGUGGGUAA | GKSALTIQLIQNHFV |
| | | GUCUGCCUUGACCAUCCAGCUGAUC | DE (SEQ ID NO: 504) |
| | | CAGAACCAUUUCGUUGAUGAA (SEQ | |
| | | ID NO: 248) | |
| 3-G12S-4 | 8 | GUCGGCGCCUCGGGCGUGGGGAAA | VGASGVGK (SEQ ID |
| | | (SEQ ID NO: 249) | NO: 505) |

Table 21 shows the variant peptides contained in Antigen 20 and their mRNA sequences.

TABLE 21

| | Length | | |
|---|---|---|---|
| Peptide name | (aa) | mRNA | Peptide |
| 11-G12D-19 | 31 | AUGACAGAGUACAAACUGGUAGUAG | DE(SEQ ID NO: 506) |
| | | UGGGAGCAGAUGGGGUUGGCAAAU | MTEYKLVVVGADGV |
| | | CCGCGCUCACCAUCCAGCUGAUUCA | GKSALTIQLIQNHFV |
| | | AAACCAUUUCGUCGAUGAG (SEQ ID | |
| | | NO: 250) | |
| 3-G12D-4 | 8 | GUGGGCGCUGACGGGGUGGGCAAG | VGADGVGK (SEQ ID |
| | | (SEQ ID NO: 251) | NO: 507) |
| 11-G12V-19 | 31 | AUGACCGAGUAUAAACUCGUAGUGG | MTEYKLVVVGAVGV |
| | | UCGGAGCUGUGGGAGUGGGAAAGU | GKSALTIQLIQNHFV |
| | | CAGCGCUCACUAUCCAACUAAUACA | DE (SEQ ID NO: 508) |
| | | GAAUCACUUUGUCGACGAA (SEQ ID | |
| | | NO: 252) | |
| 3-G12V-4 | 8 | GUGGGCGCCGUGGGUGUCGGCAAG | VGAVGVGK (SEQ ID |
| | | (SEQ ID NO: 253) | NO: 509) |
| 11-G12R-19 | 31 | AUGACAGAAUACAAGUUGGUCGUUG | MTEYKLVVVGARGV |
| | | UUGGUGCCCGCGGAGUGGGCAAAU | GKSALTIQLIQNHFV |
| | | CUGCACUGACCAUCCAACUGAUCCA | DE (SEQ ID NO: 510) |
| | | GAACCACUUUGUGGAUGAG (SEQ ID | |
| | | NO: 254) | |
| 3-G12R-4 | 8 | GUCGGUGCCAGGGGUGUGGGAAAG | VGARGVGK (SEQ ID |
| | | (SEQ ID NO: 255) | NO: 511) |
| 11-Q61H-19 | 31 | ACAUGUUUGUUAGACAUUCUCGACA | TCLLDILDTAGHEEY |
| | | CGGCUGGGCAUGAGGAGUAUAGCG | SAMRDQYMRTGEG |
| | | CUAUGCGGGAUCAGUAUAUGAGAAC | FLC (SEQ ID NO: |
| | | CGGCGAAGGCUUUCUCUGC (SEQ ID | 512) |
| | | NO: 256) | |
| 3-Q61H-4 | 8 | ACAGCCGGGCACGAGGAAUAUUCC | TAGHEEYS (SEQ ID |
| | | (SEQ ID NO: 257) | NO: 513) |
| 11-Q61R-19 | 31 | ACUUGCUUGCUGGACAUAUUAGACA | TCLLDILDTAGREEY |
| | | CAGCAGGGAGGGAAGAGUAUUCCG | SAMRDQYMRTGEG |
| | | CAAUGCGAGAUCAGUACAUGCGUAC | FLC (SEQ ID NO: |
| | | UGGGGAAGGGUUCCUGUGC (SEQ ID | 514) |
| | | NO: 258) | |
| 3-Q61R-4 | 8 | ACCGCCGGGAGGGAGGAAUACAGC | TAGREEYS (SEQ ID |
| | | (SEQ ID NO: 259) | NO: 515) |
| 11-G13C-19 | 31 | ACUGAGUACAAGCUGGUUGUGGUG | TEYKLVVVGAGCVG |
| | | GGAGCCGGAUGUGUCGGUAAAAGU | KSALTIQLIQNHFVD |
| | | GCCCUAACCAUCCAGCUCAUUCAGA | EY (SEQ ID NO: 516) |
| | | ACCACUUCGUCGAUGAGUAU (SEQ | |
| | | ID NO: 260) | |
| 3-G13C-4 | 8 | GGGGCCGGCUGUGUUGGGAAAAGC | GAGCVGKS (SEQ ID |
| | | (SEQ ID NO: 261) | NO: 517) |
| 11-G12C-19 | 31 | AUGACUGAGUACAAGCUCGUUGUCG | MTEYKLVVVGACGV |
| | | UGGGUGCAUGCGGAGUAGGGAAGU | GKSALTIQLIQNHFV |
| | | CAGCGCUGACAAUUCAGUUAAUCCA | DE (SEQ ID NO: 518) |
| | | GAAUCACUUCGUCGAUGAA (SEQ ID | |
| | | NO: 262) | |
| 3-G12C-4 | 8 | GUCGGCGCCUGUGGAGUGGGUAAG | VGACGVGK (SEQ ID |
| | | (SEQ ID NO: 263) | NO: 519) |
| 11-G13D-19 | 31 | ACGGAGUACAAGCUGGUCGUGGUU | TEYKLVVVGAGDVG |
| | | GGCGCCGGCGACGUGGGCAAAAGC | KSALTIQLIQNHFVD |
| | | GCUUUGACCAUUCAACUGAUCCAGA | EY (SEQ ID NO: 520) |
| | | AUCAUUUUGUGGAUGAAUAC (SEQ | |
| | | ID NO: 264) | |
| 3-G13D-4 | 8 | GGGGCAGGGGACGUGGGGAAAUCU | GAGDVGKS (SEQ ID |
| | | (SEQ ID NO: 265) | NO: 521) |

Table 22 shows the variant peptides contained in Antigen 21 and their mRNA sequences.

TABLE 22

| | Length | | |
|---|---|---|---|
| Peptide name | (aa) | mRNA | Peptide |
| 11-G12D-19 | 31 | AUGACUGAAUAUAAAUUAGUUGUGG | MTEYKLVVVGADGV |
| | | UGGGGAGCCGAUGGAGUGGGGAAA | GKSALTIQLIQNHFV |
| | | UCCGCGUUGACAAUCCAGCUGAUCC | DE (SEQ ID NO: 522) |
| | | AGAACCAUUUUGUUGACGAA (SEQ | |
| | | ID NO: 266) | |
| 3-G12D-4 | 8 | GUUGGAGCUGAUGGCGUUGGCAAG | VGADGVGK (SEQ ID |
| | | (SEQ ID NO: 267) | NO: 523) |
| 11-G12V-19 | 31 | AUGACGGAAUACAAGCUGGUCGUCG | MTEYKLVVVGAVGV |
| | | UGGGCGCGGUCGGGGUAGGUAAGU | GKSALTIQLIQNHFV |
| | | CCGCGCUGACAAUUCAGCUGAAUUC | DE (SEQ ID NO: 524) |
| | | AGAAUCACUUCGUGGAUGAG (SEQ | |
| | | ID NO: 268) | |
| 3-G12V-4 | 8 | GUGGGCGCCGUGGGAGUCGGUAAG | VGAVGVGK (SEQ ID |
| | | (SEQ ID NO: 269) | NO: 525) |
| 11-G13D-19 | 31 | ACCGAGUACAAGCUCGUGGUUGUC | TEYKLVVVGAGDVG |
| | | GGUGCUGGUGACGUGGGCAAGUCG | KSALTIQLIQNHFVD |
| | | GCUCUGACGAUCCAACUGAUCCAGA | EY (SEQ ID NO: 526) |
| | | AUCAUUUCGUUGAUGAGUAC (SEQ | |
| | | ID NO: 270) | |
| 3-G13D-4 | 8 | GGGGCCGGAGAUGUCGGCAAAUCU | GAGDVGKS (SEQ ID |
| | | (SEQ ID NO: 271) | NO: 527) |
| 11-A146T-19 | 31 | JAGGUCAUACGGGAUACCUUUCAUUG | RSYGIPFIETSTKTR |
| | | AGACUUCCACCAAAACUCGUCAACG | QRVEDAFYTLVREIR |
| | | CGUCGAGGAUGCCUUUUAUACCCUA | Q (SEQ ID NO: 528) |
| | | GUGAGAGAGAUCCGACAG (SEQ ID | |
| | | NO: 272) | |
| 3-A146T-4 | 8 | GAAACAAGCACCAAGACCCGGCAG | ETSTKTRQ (SEQ ID |
| | | (SEQ ID NO: 273) | NO: 529) |
| 11-G12C-19 | 31 | AUGACAGAAUACAAGUUGGUAGUUG | MTEYKLVVVGACGV |
| | | UCGGGCCCUGCGGGGUGGGGAAAA | GKSALTIQLIQNHFV |
| | | GUGCUCUAACCAUUCAAUUAAUCCA | DE (SEQ ID NO: 530) |
| | | AAAUCACUUUGUCGACGAG (SEQ ID | |
| | | NO: 274) | |
| 3-G12C-4 | 8 | GUGGGCGCCUGCGGGGUAGGCAAA | VGACGVGK (SEQ ID |
| | | (SEQ ID NO: 275) | NO: 531) |
| 11-G12A-19 | 31 | AUGACCGAAUAUAAACUGGUGGUGG | MTEYKLVVVGAAGV |
| | | UGGGAGCUGCAGGAGUUGGAAAAA | GKSALTIQLIQNHFV |
| | | GCGCUCUGACUAUCCAACUGAUUCA | DE (SEQ ID NO: 532) |
| | | GAACCAUUUCGUGGACGAG (SEQ ID | |
| | | NO: 276) | |
| 3-G12A-4 | 8 | GUCGGGGCAGCCGGCGUGGGAAAA | VGAAGVGK (SEQ ID |
| | | (SEQ ID NO: 277) | NO: 533) |
| 11-G12S-19 | 31 | AUGACCGAAUACAAGCUGGUGGUG | MTEYKLVVVGASGV |
| | | GUUGGCGCAUCCGGUGUGGGAAAG | GKSALTIQLIQNHFV |
| | | UCAGCCCUGACAAUCCAGCUUAUAC | DE (SEQ ID NO: 534) |
| | | AGAACCACUUCGUCGACGAA (SEQ | |
| | | ID NO: 278) | |
| 3-G12S-4 | 8 | GUGGGCGCAUCUGGAGUCGGAAAG | VGASGVGK (SEQ ID |
| | | (SEQ ID NO: 279) | NO: 535) |
| 11-G13C-19 | 31 | ACAGAGUAUAAAUUGGUGGUGGUAG | TEYKLVVVGAGCVG |
| | | GUGCUGGAUGUGUCGGCAAGUCCG | KSALTIQLIQNHFVD |
| | | CACUUACUAUACAGUCUCAUUCAGA | EY (SEQ ID NO: 536) |
| | | ACCACUUUGUAGACGAGUAU (SEQ | |
| | | ID NO: 280) | |
| 3-G13C-4 | 8 | GGGGCAGGGUGCGUAGGCAAGUCC | GAGCVGKS (SEQ ID |
| | | (SEQ ID NO: 281) | NO: 537) |

Table 23 shows the variant peptides contained in Antigen 22 and their mRNA sequences.

TABLE 23

| | Length | | |
|---|---|---|---|
| Peptide name | (aa) | mRNA | Peptide |
| 11-G12D-19 | 31 | AUGACGGAAUAUAAGUUGGUCAUGG | MTEYKLVMVGADG |
| | | UGGGUGCGGAUGGUGUAGGCAAAU | VGKSALTIQLIQNHF |
| | | CAGCUCUGACUAUCCAACUGAUCCA | VDE (SEQ ID NO: |
| | | GAAUCACUUUGUGGACGAA (SEQ ID | 538) |
| | | NO: 282) | |
| 3-G12D-4 | 8 | GUCGGGGCUGACGGUGUCGGAAAA | VGADGVGK (SEQ ID |
| | | (SEQ ID NO: 283) | NO: 539) |
| 11-G12C-19 | 31 | AUGACCGAAUAUAAGCUCGUUGUGG | MTEYKLVVVGACGV |
| | | UGGGGGCUUGUGGAGUUGGCAAGU | GKSALTIQLIQNHFV |
| | | CCGCUCUGACUAUCCAACUAAUUCA | DE (SEQ ID NO: 540) |
| | | GAAUCAUUUCGUCGAUGAG (SEQ ID | |
| | | NO: 284) | |
| 3-G12C-4 | 8 | GUCGGCGCUUGCGGAGUAGGCAAA | VGACGVGK (SEQ ID |
| | | (SEQ ID NO: 285) | NO: 541) |
| 11-G12V-19 | 31 | AUGACCGAAUACAAGCUCGUGGUGG | MTEYKLVVVGAVGV |
| | | UAGGCGCCGUUGGUGUAGGGAAGA | GKSALTIQLIQNHFV |
| | | GUGCGCUAACCAUUCAACUUAUCCA | DE (SEQ ID NO: 542) |
| | | GAACCAUUUUGUUGACGAG (SEQ ID | |
| | | NO: 286) | |
| 3-G12V-4 | 8 | GUAGGCGCGGUGGGAGUGGGCAAG | VGAVGVGK (SEQ ID |
| | | (SEQ ID NO: 287) | NO: 543) |
| 11-G12R-19 | 31 | AUGACCGAGUAUAAACUUGUAGUGG | MTEYKLVVVGARGV |
| | | UGGGCGCCAGGGGCGUGGGGAAAA | GKSALTIQLIQNHFV |
| | | GCGCCCUGACAAUCCAGCUCAUACA | DE (SEQ ID NO: 544) |
| | | GAAUCACUUCGUAGACCGAG (SEQ | |
| | | ID NO: 288) | |
| 3-G12R-4 | 8 | GUGGGCGCCCGCGGCGUUGGUAAG | VGARGVGK (SEQ ID |
| | | (SEQ ID NO: 289) | NO: 545) |
| 11-G13C-19 | 31 | ACUGAAUAUAAACUCGUCGUCGUGG | TEYKLVVVGAGCVG |
| | | GAGCAGGCUGUGUGGGGAAGAGUG | KSALTIQLIQNHFVD |
| | | CCUUGACCAUUCAGUUGAUCCAGAA | EY (SEQ ID NO: 546) |
| | | CCACUUCGUCGACGAGUAU (SEQ ID | |
| | | NO: 290) | |
| 3-G13C-4 | 8 | GGAGCAGGGUGCGUUGGAAAGAGC | GAGCVGKS (SEQ ID |
| | | (SEQ ID NO: 291) | NO: 547) |
| 11-G13D-19 | 31 | ACAGAAUACAAACUGGUUGUCGUGG | TEYKLVVVGAGDVG |
| | | GGGCCGGCGACGUGGGCAAGAGCG | KSALTIQLIQNHFVD |
| | | CCUUGACUAUUCAACUCAUACAGAA | EY (SEQ ID NO: 548) |
| | | CCACUUUGUGGAUGAGUAC (SEQ ID | |
| | | NO: 292) | |
| 3-G13D-4 | 8 | GGCGCCGGGGACGUGGGGAAAAGC | GAGDVGKS (SEQ ID |
| | | (SEQ ID NO: 293) | NO: 549) |
| 11-Q61H-19 | 31 | ACGUGUCUUCUGGAUAUUUUAGACA | TCLLDILDTAGHEEY |
| | | CCGCAGGGCAUGAGGAGUACUCCG | SAMRDQYMRTGEG |
| | | CAAUGCGAGAUCAGUACAUGAGGAC | FLC (SEQ ID NO: |
| | | AGGUGAGGGUUUCCUGUGC (SEQ ID | 550) |
| | | NO: 294) | |
| 3-Q61H-4 | 8 | ACAGCCGGCCACGAGGAAUACUCU | TAGHEEYS (SEQ ID |
| | | (SEQ ID NO: 295) | NO: 551) |

### Example 2: Cloning of a Sequence Encoding an Antigenic Peptide Linked to Multiple KRAS Variant Peptides

For cloning of a nucleotide sequence having as an open reading frame (ORF) the DNA sequence encoding an antigenic peptide linked to multiple KRAS variant peptides described in Example 1 above, a T7 promoter and a 5′-UTR sequence were added to the 5′ region of the sequence, and HindIII and Xhol restriction enzyme sequences were inserted to the 5′ and 3′ ends, respectively, to secure the sequence through gene synthesis.

The synthesized sequence contained HindIII and Xhol restriction enzyme sequences at the 5′ and 3′ ends, respectively, so that it could be used for cloning. Each synthesized sequence encoding the antigens and the template vector were digested with HindIII and Xhol, and then ligated using DNA ligase to construct a vector comprising a sequence encoding “mRNA.”

The full-length mRNAs of Antigens 1 to 22 shown in Table 1 each have the structure of 5′UTR (SEQ ID NO: 554)-ORF-stop codon-3′UTR (SEQ ID NO: 555)-polyA (SEQ ID NO: 556).

Table 24 shows the full-length mRNA sequences of Antigens 2, 6, 14, 15, and 17, which are KRAS mutant anticancer vaccine candidates used for efficacy evaluation.

TABLE 24

| | Full-length mRNA sequence | ORF |
|---|---|---|
| Candidate | AAGCTTTAATACGACTCACTATAAGTCCTCCCC | (SEQ ID NO: 2) |
| Substance | ATCCTCTCCCTCTGTCCCTCTGTCCCTCTGACC | |
| 2 | CTGCACTGTCCCAGCACCATGGATATGAGGGT | |
| | GCCCGCCCAGCTGCTGGGGCTGCTGCTGCTG | |
| | TGGCTGCGGGGCGCCCGGTGCTACAAGCTGG | |
| | TCGTCGTGGGGGCCTGCGGCGTGGGGAAGTC | |
| | CGCTCTGACCTACAAACTGGTGGTGGTGGGG | |
| | GCCGATGGGGTGGGCAAGTCCGCCCTGACCT | |
| | ACAAGCTGGTGGTGGTCGGGGCCGTCGGCGT | |
| | GGGCAAAAGCGCCCTGACATACAAGCTCGTGG | |
| | TGGTGGGCGCCAGGGGGGTGGGCAAGTCCGC | |
| | CCTGACAAAGCTGGTCGTGGTGGGCGCCGGG | |
| | TGCGTGGGCAAGAGCGCCCTGACAATCAAGCT | |
| | GGTGGTGGTGGGGGCCGGGGATGTGGGCAA | |
| | GAGCGCCCTGACCATCCTGGACATCCTGGACA | |
| | CAGCCGGGCACGAGGAGTACAGCGCCATGAG | |
| | GGATTGATGACTCGAGGTGTGTGGAGGACACC | |
| | CTGAACCCCCCGCTTTCAAACAAGTTTTCAAAT | |
| | TGTTTGAGGTCAGGATTTCTCAAACTGATTCCT | |
| | TTCTTTGCATATGAGTATTTGAAAATAAATATTT | |
| | TCCCAGAATATAAATAAATCATCACATGATTATT | |
| | TTAACTATGCTAGCAAAAAAAAAAAAAAAAAAA | |
| | AAAAAAAAAAAAAAAAAAAAAAAAAAAAAAAAAA | |
| | AAAAAAAAAAAAAAAAAAAAAAAAAAAAAAAAAA | |
| | AAAAAAAAAAAAAAGAAGAGCGAATTC (SEQ ID | |
| | NO: 296) | |
| Candidate | AAGCTTTAATACGACTCACTATAAGTCCTCCCC | (SEQ ID NO: 6) |
| Substance | ATCCTCTCCCTCTGTCCCTCTGTCCCTCTGACC | |
| 6 | CTGCACTGTCCCAGCACCATGGATATGAGAGT | |
| | GCCCGCCCAACTGCTGGGGCTGCTGCTGCTG | |
| | TGGCTGAGAGGCGCGAGATGCATGACAGAATA | |
| | CAAACTTGTGGTAGTTGGAGCATGCGGTGTGG | |
| | GTAAGAGTGCCCTCACCATCCAACTAATTCAGA | |
| | ACCACTTCGTTGACGAACTCCTCTCAGTGGGG | |
| | GGGGTTGGAGCATGTGGTGTAGGGAAGCTGTT | |
| | GAGCGTCGGCGGGATGACTGAATACAAACTTG | |
| | TGGTTGTTGGAGCAGATGGGGTCGGAAAATCC | |
| | GCCTTGACAATACAGCTTATTCAGAATCACTTT | |
| | GTGGATGAGCTGTTAAGCGTTGGCGGCGTCG | |
| | GAGCTGATGGCGTCGGAAAGCTCTTGTCTGTG | |
| | GGCGGCACTGAGTATAAATTAGTGGTAGTCGG | |
| | GGCTGGAGACGTGGGCAAGAGTGCCCTTACC | |
| | ATCCAGCTCATCCAGAATCATTTTGTGGACGAG | |
| | TATCTATTGTCCGTGGGAGGTGGAGCTGGTGA | |
| | CGTCGGAAAGTCTTAATGACTCGAGGTGTGTG | |
| | GAGGACACCCTGAACCCCCCGCTTTCAAACAA | |
| | GTTTTCAAATTGTTTGAGGTCAGGATTTCTCAA | |
| | ACTGATTCCTTTCTTTGCATATGAGTATTTGAAA | |
| | ATAAATATTTTCCCAGAATATAAATAAATCATCA | |
| | CATGATTATTTTAACTATGCTAGCAAAAAAAAAA | |
| | AAAAAAAAAAAAAAAAAAAAAAAAAAAAAAAAAA | |
| | AAAAAAAAAAAAAAAAAAAAAAAAAAAAAAAAAA | |
| | AAAAAAAAAAAAAAAAAAAAAAAGAAGAGCGAA | |
| | TTC (SEQ ID NO: 297) | |
| Candidate | AAGCTTTAATACGACTCACTATAAGAATCCAGT | (SEQ ID NO: |
| Substance | CAACGCATATCCACGTATACCCGAACACATCA | 14) |
| 14 | CAGCCACCATGGATATGCGAGTGCCCGCTCAG | |
| | CTTCTGGGGTTGCTTCTGTTATGGCTGAGGGG | |
| | GGCCCGGTGTATGACAGAATACAAGTTAGTGG | |
| | TGGTAGGAGCGTGTGGAGTGGGGAAAAGCGC | |
| | CCTAACTATTCAGTTGATACAAAACCATTTCGT | |
| | GGACGAAGTGGGCGCATGTGGAGTCGGCAAG | |
| | ATGACCGAGTATAAGCTCGTAGTCGTGGGGGC | |
| | CGACGGCGTGGGGAAATCAGCCCTTACTATCC | |
| | AGCTTATACAAAACCATTTTGTTGACGAAGTAG | |
| | GCGCCGACGGGGTCGGAAAGATGACCGAGTA | |
| | CAAGCTGGTTGTCGTCGGGGCCGTAGGGGTT | |
| | GGCAAAAGCGCCCTGACTATCCAGCTCATCCA | |
| | AAATCATTTCGTCGACGAGGTGGGCGCCGTTG | |
| | GAGTCGGTAAAATGACGGAATACAAGTTGGTG | |
| | GTAGTGGGGGCTCGGGGGGTGGGCAAGAGTG | |
| | CTTTGACCATACAGTTAATTCAGAACCACTTTG | |
| | TGGATGAGGTCGGCGCTCGCGGAGTCGGCAA | |
| | AACCGAGTATAAATTGGTTGTTGTCGGCGCAG | |
| | GCTGTGTGGGCAAGTCTGCTCTCACCATTCAA | |
| | CTAATCCAGAACCACTTTGTTGATGAGTACGGA | |
| | GCAGGCTGCGTGGGAAAAAGCACGGAGTACA | |
| | AGCTGGTTGTCGTAGGAGCAGGAGACGTGGG | |
| | CAAATCCGCTCTGACAATTCAGCTTATCCAGAA | |
| | TCACTTTGTGGATGAATATGGTGCCGGGGATG | |
| | TCGGTAAAAGTACCTGCTTACTGGACATCCTG | |
| | GACACTGCTGGTCACGAGGAATACTCCGCGAT | |
| | GCGAGATCAGTATATGCGGACAGGAGAGGGTT | |
| | TCCTGTGCACAGCAGGCCATGAGGAATATTCT | |
| | ACTGAATATAAGCTGGTGGTGGTGGGGGCTGG | |
| | TAGAGTGGGGAAGTCAGCACTGACAATCCAGC | |
| | TCATTCAGAATCACTTCGTTGATGAGTACGGTG | |
| | CGGGTAGAGTGGGCAAGTCCTAATGACTCGAG | |
| | GTGTGTGGAGGACACCCTGAACCCCCCGCTTT | |
| | CAAACAAGTTTTCAAATTGTTTGAGGTCAGGAT | |
| | TTCTCAAACTGATTCCTTTCTTTGCATATGAGTA | |
| | TTTGAAAATAAATATTTTCCCAGAATATAAATAA | |
| | ATCATCACATGATTATTTTAACTATGCTAGCAAA | |
| | AAAAAAAAAAAAAAAAAAAAAAAAAAAAAAAAAA | |
| | AAAAAAAAAAAAAAAAAAAAAAAAAAAAAAAAAA | |
| | AAAAAAAAAAAAAAAAAAAAAAAAAAAAAAGAA | |
| | GAGCGAATTC (SEQ ID NO: 298) | |
| Candidate | AAGCTTTAATACGACTCACTATAAGAATCCAGT | (SEQ ID NO: |
| Substance | CAACGCATATCCACGTATACCCGAACACATCA | 15) |
| 15 | CAGCCACCATGGATATGCGAGTGCCCGCTCAG | |
| | CTTCTGGGGTTGCTTCTGTTATGGCTGAGGGG | |
| | GGCCCGGTGTATGACGGAATACAAGCTCGTCG | |
| | TGGTGGGCGCCGATGGCGTCGGCAAGTCGGC | |
| | ACTGACTATCCAGCTCATACAAAATCATTTTGT | |
| | TGATGAACTTTTATCCGTCGGTGGAGTGGGGG | |
| | CGGACGGAGTGGGGAAGCTGCTGAGCGTTGG | |
| | TGGGATGACCGAATACAAACTGGTGGTGGTCG | |
| | GGGCGTGTGGGGTGGGCAAATCAGCCCTCAC | |
| | AATACAGCTGATTCAGAACCACTTTGTTGACGA | |
| | GCTTCTGTCCGTGGGGGGGGTGGGGGCATGT | |
| | GGAGTGGGGAAGCTCCTGTCCGTAGGGGGCA | |
| | TGACTGAGTACAAGCTGGTTGTTGTAGGTGCT | |
| | GTTGGAGTGGGAAAGAGCGCCCTCACAATCCA | |
| | ATTGATCCAGAATCACTTCGTCGACGAGCTCTT | |
| | GAGCGTTGGAGGTGTGGGGGCCGTAGGCGTG | |
| | GGCAAGCTGCTATCTGTGGGTGGAATGACCGA | |
| | ATATAAGTTGGTAGTGGTGGGGGCTCGCGGAG | |
| | TCGGGAAGAGTGCTCTTACTATTCAGCTGATTC | |
| | AGAACCATTTCGTAGATGAACTCCTATCAGTCG | |
| | GTGGCGTGGGCGCCAGAGGCGTGGGCAAACT | |
| | GTTATCAGTGGGCGGTACTGAGTACAAACTAG | |
| | TAGTCGTAGGAGCTGGGTGTGTGGGCAAAAGT | |
| | GCACTCACCATCCAGCTGATCCAGAACCATTT | |
| | CGTGGATGAGTATTTGTTGAGTGTCGGAGGTG | |
| | GAGCTGGCTGCGTGGGCAAAAGCCTGCTGTC | |
| | CGTTGGGGGGACGGAATACAAACTAGTTGTTG | |
| | TGGGGGCTGGTGATGTCGGAAAGTCCGCCTT | |
| | GACTATTCAGCTTATTCAAAATCACTTTGTCGA | |
| | CGAGTATCTTCTGAGCGTCGGCGGCGGCGCA | |
| | GGTGACGTAGGAAAGTCTTTACTGTCAGTTGG | |
| | GGGAACCTGCCTTCTGGACATCCTGGACACAG | |
| | CCGGCCACGAGGAATACAGCGCCATGCGGGA | |
| | TCAGTATATGAGGACAGGAGAAGGCTTCCTCT | |
| | GCCTCTTAAGTGTCGGCGGTACAGCTGGCCAC | |
| | GAGGAGTATTCTCTGCTGTCTGTCGGAGGCAC | |
| | CGAGTACAAACTTGTGGTGGTTGGAGCAGGGC | |
| | GTGTCGGCAAATCTGCCCTGACCATCCAGCTG | |
| | ATACAAAACCATTTTGTGGATGAGTATTTACTTT | |
| | CCGTGGGAGGCGGTGCGGGACGAGTGGGCAA | |
| | GAGCTAATGACTCGAGGTGTGTGGAGGACACC | |
| | CTGAACCCCCCGCTTTCAAACAAGTTTTCAAAT | |
| | TGTTTGAGGTCAGGATTTCTCAAACTGATTCCT | |
| | TTCTTTGCATATGAGTATTTGAAAATAAATATTT | |
| | TCCCAGAATATAAATAAATCATCACATGATTATT | |
| | TTAACTATGCTAGCAAAAAAAAAAAAAAAAAAA | |
| | AAAAAAAAAAAAAAAAAAAAAAAAAAAAAAAAAA | |
| | AAAAAAAAAAAAAAAAAAAAAAAAAAAAAAAAAA | |
| | AAAAAAAAAAAAAAGAAGAGCGAATTC (SEQ ID | |
| | NO: 299) | |
| Candidate | AAGCTTTAATACGACTCACTATAAGAATCCAGT | (SEQ ID NO: |
| Substance | CAACGCATATCCACGTATACCCGAACACATCA | 17) |
| 17 | CAGCCACCATGGACATGAGGGTGCCCGCCCA | |
| | GTTGCTGGGGCTGCTGCTCTTGTGGCTCAGAG | |
| | GGGCACGGTGCATGACGGAATACAAGCTCGTC | |
| | GTGGTGGGCGCCGATGGCGTCGGCAAGTCGG | |
| | CACTGACTATCCAGCTCATACAAAATCATTTTG | |
| | TTGATGAACTTTTATCCGTCGGTGGAGTGGGG | |
| | GCGGACGGAGTGGGGAAGCTGCTGAGCGTTG | |
| | GTGGGATGACCGAATACAAACTGGTGGTGGTC | |
| | GGGGCGTGTGGGGTGGGCAAATCAGCCCTCA | |
| | CAATACAGCTGATTCAGAACCACTTTGTTGACG | |
| | AGCTTCTGTCCGTGGGGGGGGTGGGGGCATG | |
| | TGGAGTGGGGAAGCTCCTGTCCGTAGGGGGC | |
| | ATGACTGAGTACAAGCTGGTTGTTGTAGGTGC | |
| | TGTTGGAGTGGGAAAGAGCGCCCTCACAATCC | |
| | AATTGATCCAGAATCACTTCGTCGACGAGCTCT | |
| | TGAGCGTTGGAGGTGTGGGGGCCGTAGGCGT | |
| | GGGCAAGCTGCTATCTGTGGGTGGAATGACCG | |
| | AATATAAGTTGGTAGTGGTGGGGGCTCGCGGA | |
| | GTCGGGAAGAGTGCTCTTACTATTCAGCTGATT | |
| | CAGAACCATTTCGTAGATGAACTCCTATCAGTC | |
| | GGTGGCGTGGGCGCCAGAGGCGTGGGCAAAC | |
| | TGTTATCAGTGGGCGGTACTGAGTACAAACTA | |
| | GTAGTCGTAGGAGCTGGGTGTGTGGGCAAAA | |
| | GTGCACTCACCATCCAGCTGATCCAGAACCAT | |
| | TTCGTGGATGAGTATTTGTTGAGTGTCGGAGG | |
| | TGGAGCTGGCTGCGTGGGCAAAAGCCTGCTG | |
| | TCCGTTGGGGGGACGGAATACAAACTAGTTGT | |
| | TGTGGGGGCTGGTGATGTCGGAAAGTCCGCC | |
| | TTGACTATTCAGCTTATTCAAAATCACTTTGTCG | |
| | ACGAGTATCTTCTGAGCGTCGGCGGCGGCGC | |
| | AGGTGACGTAGGAAAGTCTTTACTGTCAGTTG | |
| | GGGGAACCTGCCTTCTGGACATCCTGGACACA | |
| | GCCGGCCACGAGGAATACAGCGCCATGCGGG | |
| | ATCAGTATATGAGGACAGGAGAAGGCTTCCTC | |
| | TGCCTCTTAAGTGTCGGCGGTACAGCTGGCCA | |
| | CGAGGAGTATTCTTAATGACTCGAGGTGTGTG | |
| | GAGGACACCCTGAACCCCCCGCTTTCAAACAA | |
| | GTTTTCAAATTGTTTGAGGTCAGGATTTCTCAA | |
| | ACTGATTCCTTTCTTTGCATATGAGTATTTGAAA | |
| | ATAAATATTTTCCCAGAATATAAATAAATCATCA | |
| | CATGATTATTTTAACTATGCTAGCAAAAAAAAAA | |
| | AAAAAAAAAAAAAAAAAAAAAAAAAAAAAAAAAA | |
| | AAAAAAAAAAAAAAAAAAAAAAAAAAAAAAAAAA | |
| | AAAAAAAAAAAAAAAAAAAAAAAGAAGAGCGAA | |
| | TTC (SEQ ID NO: 300) | |

### Example 3: Evaluation of Anticancer Efficacy of mRNA Encoding Antigen 2 Linked to KRAS Variant Peptide in Lewis Lung Cancer Mouse Model

The anticancer efficacy of a vaccine containing mRNA encoding Antigen 2 linked to KRAS variant peptide according to the present disclosure was evaluated. The vaccine containing the mRNA encoding Antigen 2 was administered to a Lewis lung carcinoma (LL/2) syngeneic mouse model, in which Lewis lung carcinoma cells (LL/2) had been allografted, and antitumor efficacy was assessed by observing tumor size changes following administration. LL/2 cells are a murine lung cancer cell line established from the lungs of C57BL mice bearing tumors induced by implantation of primary Lewis lung cancer.

The administration group was administered the vaccine at a dose of 30 μg/head/week per subject for 5 weeks. The mRNA-containing vaccine was formulated to be loaded into lipid nanoparticles (SNPs) in PBS. The lipid nanoparticles (LNPs) contained ionizable lipid (ALC0315, 46.3 mol %), phospholipid (DSPC, 9.4 mol %), cholesterol (42.7 mol %), and PEG-lipid (ALC0159, 1.6 mol %) in PBS. The concentration of the loaded mRNA was 0.3 mg/mL. The same formulation of the mRNA-containing vaccine was used throughout the examples described herein. Specifically, after the first administration, LL/2 cells (ATCC), cultured in DMEM medium in a cell culture flask, were subcutaneously injected into the dorsal side of C57BL/6 female mice (6 weeks old, average body weight 18 g, Daehan Bio). A few days later, when tumors became visible to the naked eye, tumor size was monitored over time. Each group was assigned to seven animals with similar body weight. The administration group received 50 μl of the vaccine at a dose of 15 μg/head via intramuscular injection twice a week.

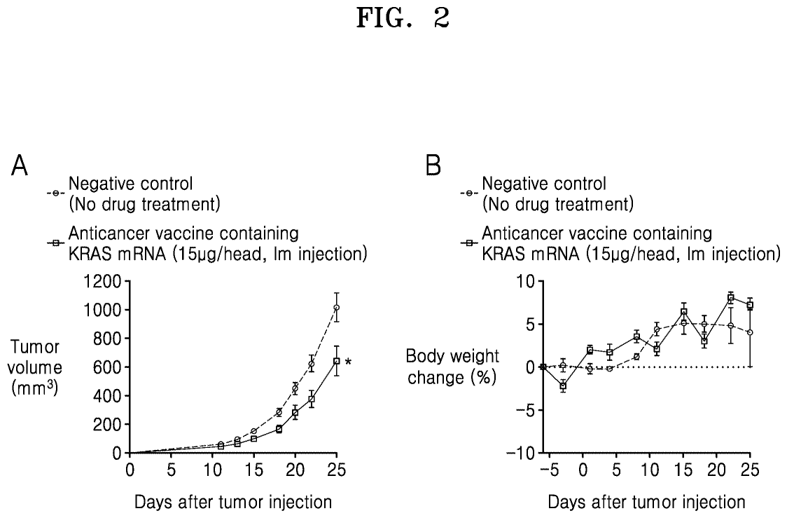

The tumor size of each group was observed for up to Day 26 from the date of tumor injection. FIG. 2 is a diagram showing tumor size and body weight changes after administration of an anti-cancer vaccine containing mRNA encoding Antigen 2 in a Lewis lung cancer mouse model. As shown in FIG. 2, the anti-cancer vaccine containing mRNA encoding Antigen 2 exhibited a significant tumor growth inhibition effect compared to the vehicle-treated group in the Lewis lung cancer mouse model (FIG. 2A). In addition, there was no decrease in mouse body weight after continuous vaccination for 5 weeks, indicating that the vaccine was safe in terms of side effects (FIG. 2B). According to previous reports, Lewis lung tumor is known as a representative immunosuppressive tumor model in which immunosuppressive cells such as myeloid derived suppressor cells (MDSC) and tumor-associated macrophages (TAM) are mainly distributed (Viitala et al., 2019, Clin Cancer Res (2019) 25 (11): 3289-3303).

Additionally, tumors were isolated from the mice, and tumor tissue cells were separated therefrom to perform flow cytometry analysis (FACS analysis). Using flow cytometry, the proportions of CD8+ T cells (CD3+CD8+ cells), memory/effector CD8+ T cells (CD3+CD8+CD44+ cells), and regulatory T cells (regulatory T cells) Treg cells (CD3+CD8+Foxp3+ cells) in the tumor tissue cells were confirmed.

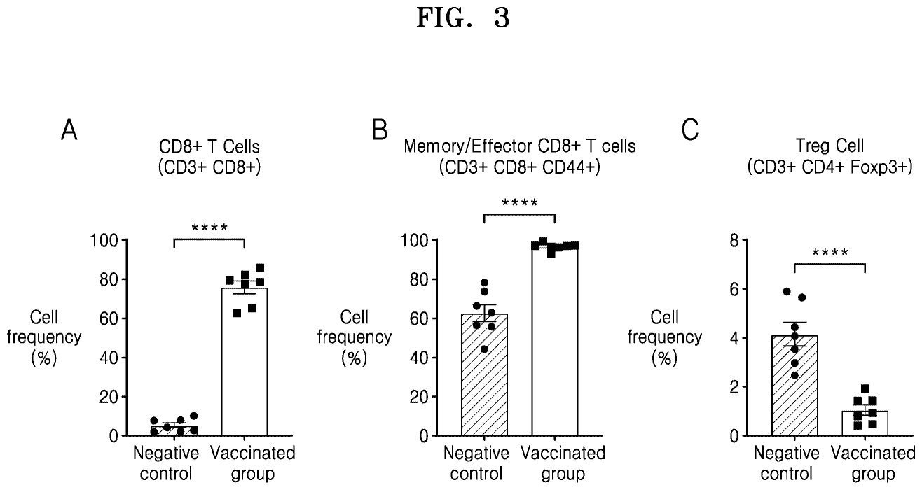

After administering an mRNA-containing anticancer vaccine to a Lewis lung cancer mouse model, changes in the distribution of immune cells among tumor tissue cells were confirmed. As a result, in the group treated with the mRNA-containing anticancer vaccine which encodes Antigen 2, the proportions of CD8+ T cells (CD3+CD8+ cells) and memory/effector CD8+ T cells (CD3+CD8+CD44+ cells) in the tumor tissue increased (FIGS. 3A and 3B), while the proportion of regulatory T cells (Treg; CD3+CD8+Foxp3+ cells), known to suppress immune responses, decreased (FIG. 3C).

These results indicate that the KRAS mRNA anticancer vaccine exerts its anticancer efficacy by promoting the proliferation of tumor-killing cells such as CD8+ T cells (CD3+CD8+ cells) and memory/effector CD8+ T cells (CD3+CD8+CD44+ cells), while suppressing the proliferation of regulatory T cells (Tregs; CD3+CD8+Foxp3+ cells), which inhibit immune responses. Moreover, the observation of anti-tumor effects even in Lewis lung tumors-which are known to be a representative immunosuppressive tumor model-suggests that the KRAS mRNA anticancer vaccine may exhibit potent anticancer efficacy regardless of the tumor immune microenvironment.

This demonstrates that the KRAS mRNA anticancer vaccine is effective in immunosuppressive tumors.

### Example 4: Evaluation of Anticancer Efficacy of mRNA Encoding Antigen 6 Linked to KRAS Variant Peptide in a Lewis Lung Cancer Mouse Model

The anticancer efficacy of an anticancer vaccine containing mRNA encoding Antigen 6 linked to a KRAS variant peptide according to the present disclosure was evaluated. After administering the mRNA-containing vaccine to a Lewis lung cancer mouse model (LL/2 tumor syngeneic mouse model), the antitumor efficacy was evaluated by observing changes in tumor size. The administration group was administered the vaccine at a dose of 20 μg/head/week per individual for 5 weeks. The mRNA-containing vaccine has the same composition as described in Example 3. Specifically, following the first administration, LL/2 cells (ATCC), cultured in DMEM medium in a cell culture flask, were subcutaneously injected into the dorsal side of C57BL/6 female mice (6 weeks old, average body weight 18 g, Daehan Bio). A few days later, when tumors became visibly apparent, tumor sizes were monitored. Each group was composed of seven mice with similar body weights. The administration group received intramuscular injections of the vaccine at a dose of 10 μg/head (50 μL per dose), twice per week.

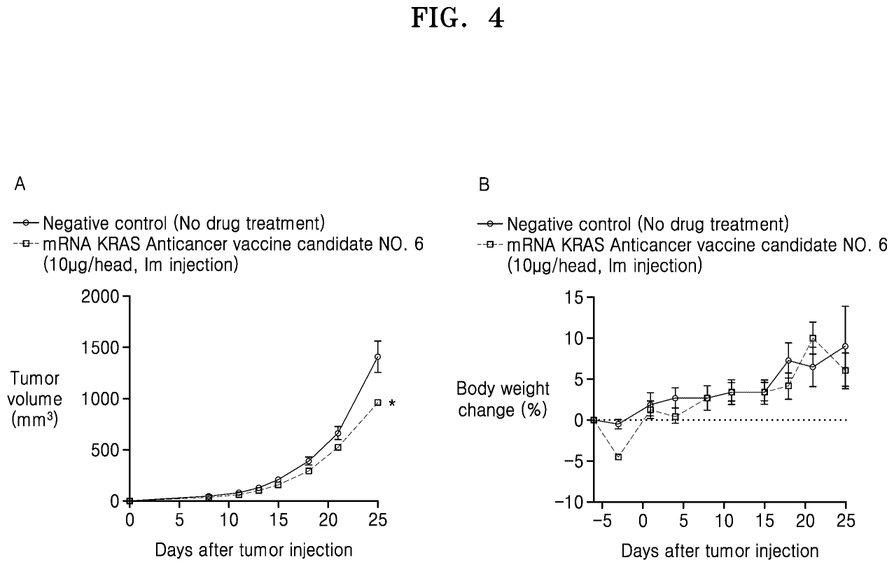

The tumor size of each group was observed for up to Days 26 from the date of tumor injection. After administering an anticancer vaccine containing mRNA encoding Antigen 6 to a Lewis lung cancer mouse model, the anticancer vaccine containing mRNA encoding antigen 6 showed significant tumor growth inhibition efficacy compared to the vehicle-administered group (FIG. 4A). Additionally, no reduction in body weight was observed in mice over the 5-week vaccination period, confirming its safety in terms of side effects (FIG. 4B).

Furthermore, flow cytometry (FACS) analysis was performed on tumors isolated from the mice, and the frequency of CD8+ T cells (CD3+CD8+ cells) within the tumor tissue was examined.

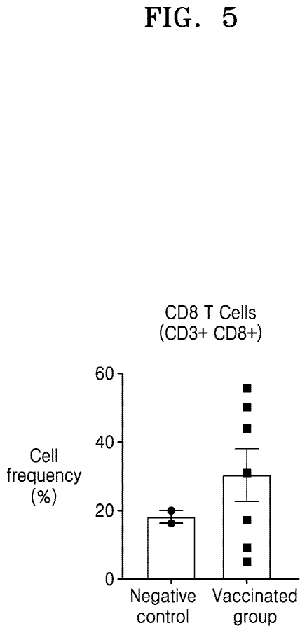

After administering an mRNA-containing anticancer vaccine to a Lewis lung cancer mouse model, changes in the distribution of immune cells among tumor tissue cells were confirmed. When an anticancer vaccine containing mRNA encoding Antigen 6 was administered, the proportion of CD8+ T cells (CD3+CD8+ cells) among tumor tissue cells increased (FIG. 5).

These results indicate that the KRAS mRNA anticancer vaccine exerts its anticancer efficacy by inducing the proliferation of cells involved in tumor killing, such as CD8+ T cells (CD3+CD8+ cells). Moreover, the observation of anti-tumor effects in Lewis lung tumors-known to be representative immunosuppressive tumor models-suggests that the KRAS mRNA anti-cancer vaccine may exhibit strong anticancer efficacy regardless of the cancer immune microenvironment.

This indicates that the KRAS mRNA anticancer vaccine is effective in immunosuppressive tumors.

### Example 5: Evaluation of Anticancer Efficacy of mRNA Encoding Antigen 14 or Antigen 15 Linked to KRAS Variant Peptide in a Lewis Lung Cancer Mouse Model

The anticancer efficacy of an anticancer vaccine containing mRNA encoding Antigen 14 or Antigen 15 linked to a KRAS variant peptide according to the disclosure was evaluated. After administering the mRNA-containing vaccine to a Lewis lung cancer mouse model (LL/2 tumor syngeneic mouse model), the antitumor efficacy was evaluated by observing changes in tumor size. The administration group was administered the above vaccine at a dose of 30 μg/head/week per subject for 5 weeks. The mRNA-containing vaccine used was the same as that described in Example 3. Specifically, after the first administration, LL/2 cells (ATCC) cultured in DMEM medium in a cell culture flask were subcutaneously injected into the dorsal side of C57BL/6 female mice (6 weeks old, average body weight 18 g; Daehan Bio). A few days later, when tumors became visible to the naked eye, tumor sizes were monitored over time. Each group was assigned to seven animals with similar body weight. The administration group received 50 μl of the vaccine at 15 μg/head per session via intramuscular injection twice a week.

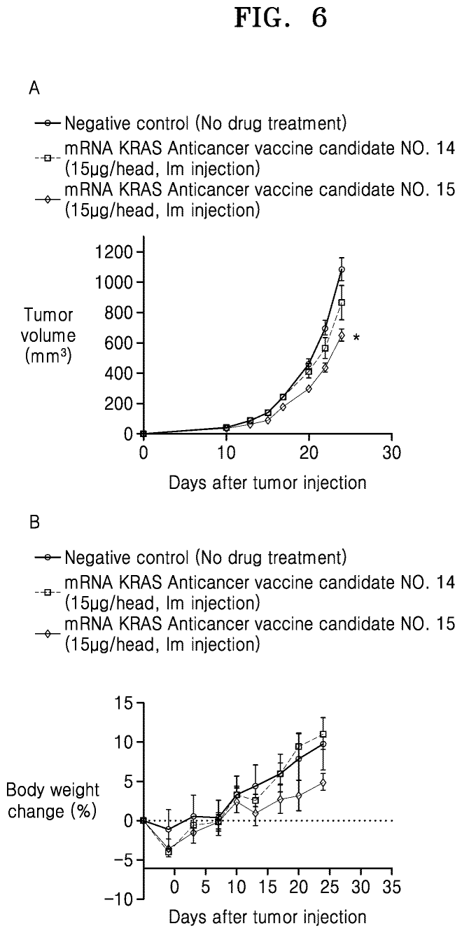

Tumor sizes in each group were monitored up to Day 26 from the day of tumor implantation. As a result, the anticancer vaccine containing mRNA of SEQ ID NO: 14 or 15 showed significant tumor growth inhibition compared to the vehicle-treated group (FIG. 6A). In addition, mRNA encoding Antigen 14 or Antigen 15 was confirmed to be safe in terms of side effects as no decrease in mouse body weight was observed even when continuous vaccination was administered for 5 weeks (FIG. 6B).

These results indicate that the anticancer vaccine containing mRNA encoding Antigen 14 or 15 exerts anticancer efficacy by inducing the proliferation of cells involved in tumor death.

Moreover, the observation of anticancer effects in Lewis lung tumors-which are known as representative immunosuppressive tumor models-suggests that the KRAS mRNA anti-cancer vaccine may demonstrate strong anti-tumor efficacy regardless of the tumor immune microenvironment.

This indicates that the KRAS mRNA anticancer vaccine is effective in immunosuppressive tumors.

### Example 6: Determination of Cell Viability During Co-Culture of mRNA-Transfected Human Peripheral Blood Mononuclear Cells and Target Cells

In this example, it was evaluated whether immune cells expressing mRNA encoding Antigens 2, 6, 15 and 17 linked to KRAS variant peptides specifically responded to target cells. To this end, a co-culture experiment was conducted between human peripheral blood mononuclear cells (PBMCs) transfected with the mRNA and target cells. The target cells included cancer cell lines expressing wild-type KRAS and cancer cell lines expressing mutant KRAS, respectively. The mRNA was synthesized through in vitro transcription. For in vivo experiments, the mRNA was delivered via LNP-loaded formulations, whereas in cell-based experiments, transfection was performed using Lipofectamine.

PBMCs were isolated from human blood collected from healthy donors using density gradient centrifugation with Histopaque®-1077 (Sigma, Cat. No. 10771). The isolated PBMCs were cultured for 2 hours in RPMI-1640 medium supplemented with 10% FBS and 1% P/S, then seeded at 1.5×10″ cells/well in 1.5 ml medium in a 6-well plate. The mRNAs encoding each KRAS variant peptide-linked antigen were mixed with Lipofectamine™ MessengerMAX™ Transfection Reagent (Thermo Scientific™, Cat. No. LMRNA001) at a ratio of 0.5 μL Lipofectamine per 1 μg mRNA and incubated for 20 minutes to form Lipofectamine/mRNA complexes. The reaction product corresponding to 5 μg of mRNA was added to the seeded PBMCs and cultured for 36 hours at 37° C. in 5% CO₂. As a result, PBMCs transfected with the respective mRNAs were obtained.

For the target cells, MRC-5 (0.5× 10ª cells/well, 10% FBS DMEM), H1299 (1×10⁴ cells/well, 10% FBS RPMI-1640), and AsPC-1 (1×10⁴ cells/well, 10% FBS RPMI-1640) were seeded into 96-well plates 24 hours prior to co-culture, allowing for adherence to the plate surface. To the adhered target cells, the mRNA-transfected PBMCs (6× 10⁴ cells/well, 10% FBS RPMI-1640) were added at effector-to-target (E:T) ratios of 1:12 or 1:6, and co-cultured for 48 hours at 37° C. in 5% CO₂.

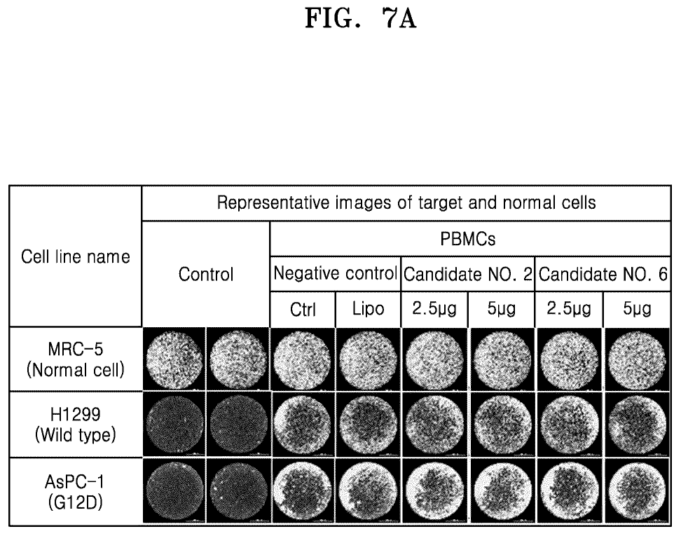

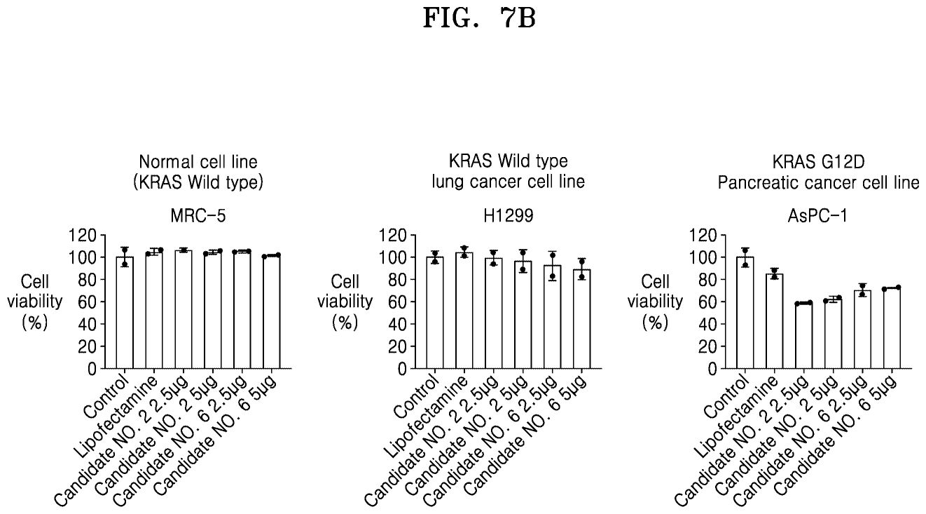

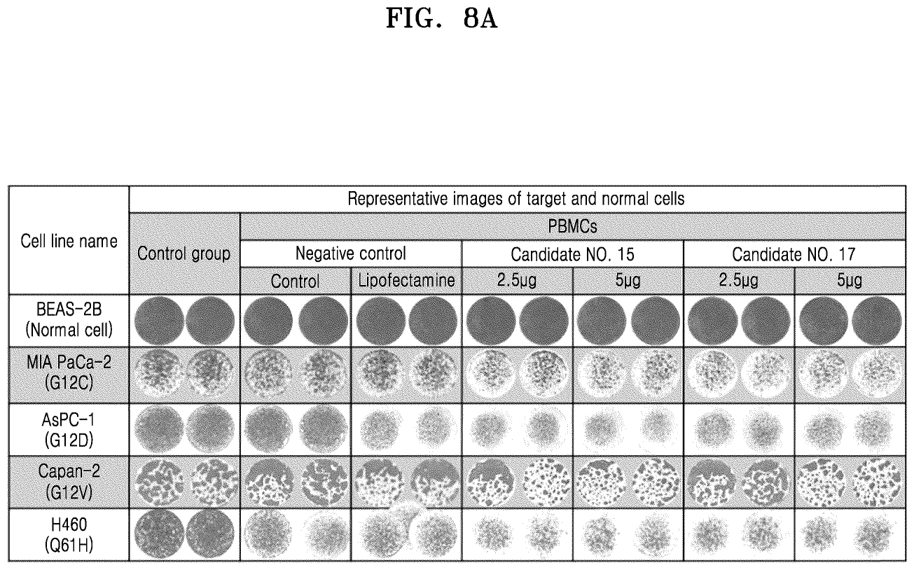

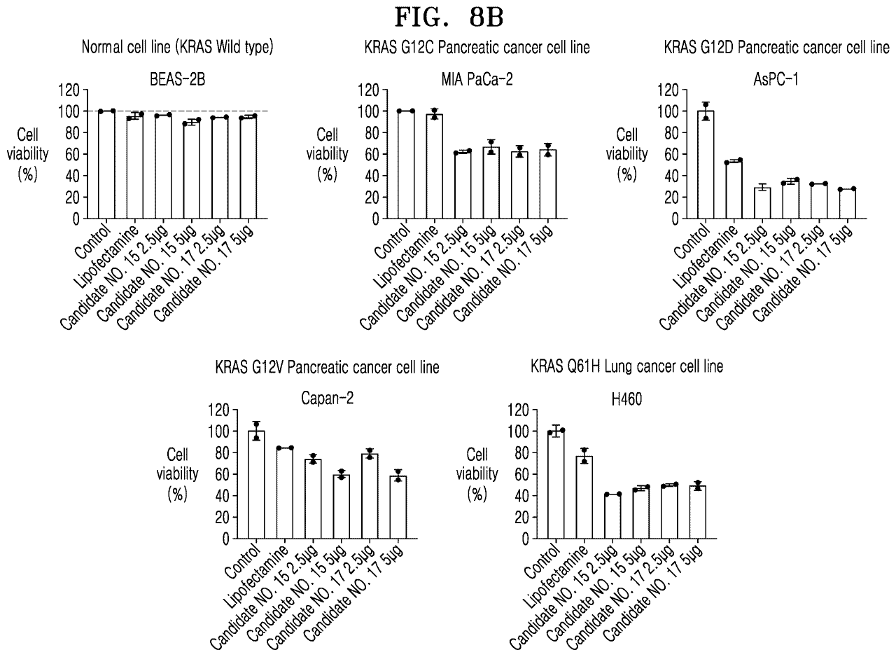

The cells were then stained with sulforhodamine B (SRB) (Sigma, Cat. No. S1402) and was stained with Cytation 5 Imaging system (Agilent, Cat No. S1402), and images were acquired using the Cytation 5 Imaging System (Agilent, Cat. No. 132BR022520). In addition, Tris (Sigma, Cat. No. 10708976001) was added to the wells to lyse the cells, and absorbance was measured at 540 nm. The absorbance at 540 nm was measured to calculate the cell viability (%) shown in FIGS. 7B and 8B.

In FIG. 7A, the “Control” refers to the result of culturing MRC-5, H1299, and AsPC-1 cells alone, without target cells. MRC-5 cells are normal cells derived from human embryonic lung fibroblasts. H1299 cells are a human non-small cell lung carcinoma cell line derived from lymph node tissue, which expresses wild-type KRAS protein. AsPC-1 cells are a nude mouse xenograft-derived cell line initiated from ascites-derived cells of a human pancreatic cancer patient, and are a pancreatic cancer cell line expressing the G12D mutant KRAS.

Additionally, among the negative controls, “Ctrl” and “Lipo” represent PBMCs not transfected with mRNA and co-cultured with PBMC cells transfected in the absence of mRNA and in the presence of Lipofectamine, respectively. In Candidate Substances 2 and 6, 2.5 μg and 5.0 μg represent cases in which transfection was performed using 2.5 μg and 5.0 μg of mRNA encoding Antigens 2 and 6, respectively.

As shown in FIG. 7A, when MRC-5 and H1299 cell lines expressing KRAS wild type were co-cultured with PBMCs transfected with mRNA encoding Antigen 2 or Antigen 6 linked to KRAS variant peptides, there was no difference in the degree of sulforhodamine B staining compared to the negative control. In contrast, when AsPC-1 cells expressing the G12D mutant protein were co-cultured with PBMCs transfected with mRNA encoding Antigen 2 or Antigen 6 linked to the KRAS variant peptide, the degree of sulforhodamine B staining was reduced compared to the negative control. The cell viability was confirmed by staining the surviving cells with sulforhodamine B and measuring the absorbance at a wavelength of 540 nm, thereby quantifying it.

As shown in FIG. 7B, when MRC-5 and H1299 cell lines expressing KRAS wild type were co-cultured with hPBMCs transfected with mRNA encoding Antigen 2 or Antigen 6 linked to KRAS variant peptides, there was no difference in the survival rate of target cells compared to the negative control group. In contrast, when AsPC-1 cells expressing the KRAS G12D mutant protein were co-cultured with PBMCs transfected with mRNA encoding Antigen 2 or Antigen 6 linked to the KRAS variant peptide, the viability of target cells was reduced compared to the negative control.

In FIG. 7B, the survival rate of target cells was calculated according to the following formula.

$$Target cell survival rate = Ti / Tc \times 100$$

Tc: Absorbance of 100% growth control target cells alone
Ti: absorbance of target cells under co-culture conditions with PBMC

The results indicate that PBMCs transfected with mRNA encoding Antigen 2 or Antigen 6 linked to KRAS variant peptides selectively inhibit the growth of cancer cells expressing the G12D mutant KRAS, i.e., AsPC-1 cells.

In FIG. 8A, the control group represents the result of co-culturing BEAS-2B, MIA PaCa-2, AsPC-1, Capan-2, and H460 cells (seeded at 1×10⁴ cells/well in 10% FBS RPMI-1640) with PBMCs (6×10⁴ cells/well in 10% FBS RPMI-1640) at a 1:6 ratio, without target cells.

BEAS-2B cells are epithelial cells derived from the bronchial epithelium of a noncancerous individual and represent a normal cell line. MIA PaCa-2 cells are an epithelial pancreatic cancer cell line derived from human pancreatic tumor tissue and express the G12C mutant form of KRAS.

AsPC-1 cells are a pancreatic cancer cell line derived from a xenograft established using ascites fluid from a human pancreatic cancer patient, and express the G12D mutant form of KRAS.

Capan-2 cells are a pancreatic cancer cell line with polygonal morphology, isolated from the pancreas of a human pancreatic cancer patient, and express the G12V mutant form of KRAS.

H460 cells are a lung cancer cell line derived from pleural fluid of a patient with large cell lung cancer, and express the Q61H mutant form of KRAS.

Additionally, among the negative controls, “Control” refers to co-culture with PBMCs that were not transfected with mRNA, and “Lipofectamine” refers to co-culture with PBMCs transfected in the absence of mRNA but in the presence of Lipofectamine. In Candidate Substances 15 and 17, 2.5 μg and 5.0 μg represent cases in which transfection was performed using 2.5 μg and 5.0 μg of mRNA encoding Antigens 15 and 17, respectively.

As shown in FIG. 8A, when BEAS-2B, a normal cell line expressing KRAS wild type, was co-cultured with PBMCs transfected with mRNA encoding Antigen 15 or 17 linked to KRAS variant peptides, there was no difference in the degree of sulforhodamine B staining compared to the negative control. In contrast, when MIA PaCa-2, AsPC-1, Capan-2, and H460 cell lines expressing KRAS G12C, G12D, G12V, and Q61H mutant proteins, respectively, were co-cultured with PBMCs transfected with mRNA encoding Antigen 15 or 17 linked to KRAS variant peptides, the degree of sulforhodamine B staining was reduced compared to the negative control.

As shown in FIG. 8B, when the normal cell line BEAS-2B cell line, which is a cell line expressing the wild type KRAS, was co-cultured with PBMCs transfected with mRNA encoding Antigen 15 or 17 linked to a KRAS variant peptide, there was no difference in the survival rate of target cells compared to the negative control group. In contrast, when MIA PaCa-2, AsPC-1, Capan-2, and H460 cell lines expressing KRAS G12C, G12D, G12V, and Q61H mutant proteins, respectively, were co-cultured with PBMCs transfected with mRNA encoding Antigen 15 or 17 linked to KRAS variant peptides, the viability of target cells decreased compared to the negative control.

In FIG. 8B, the survival rate of target cells was calculated according to the following formula.

$$Target cell survival rate = Ti / Tc \times 100$$

Tc: Absorbance of 100% growth control target cells alone
Ti: absorbance of target cells under co-culture conditions with PBMC

The results above indicate that PBMCs transfected with mRNA encoding Antigen 15 or 17 linked to KRAS variant peptides selectively inhibit the growth of only cancer cells expressing KRAS G12C, G12D, G12V, and Q61H mutant proteins, namely MIA PaCa-2, AsPC-1, Capan-2, and H460 cells.

The results in FIGS. 7A, 7B, 8A, and 8B demonstrate that immune cells, such as PBMCs, transfected with mRNA encoding an antigen linked to a KRAS variant peptide selectively inhibit only cancer cells expressing the KRAS variant peptide. Therefore, the antigen linked to the above KRAS variant peptide may be used to specifically inhibit cancer cells without acting on normal cells.

### Example 7: Confirmation of Expression of KRAS Mutant mRNA-Based Anticancer Agent in LL/2 Cells

In this example, the mRNAs encoding each KRAS variant peptide-linked antigen were delivered into LL/2 cells using Lipofectamine™ MessengerMAX™ Transfection Reagent (Thermo Scientific™, Cat. No. LMRNA001), and the expression of each antigen was confirmed. For this purpose, the following in vitro experiment was conducted. LL/2 cells were seeded in 6-well plates at a density of 4×10⁵ cells/2 ml/well and incubated for 24 hours. Each mRNA was mixed with Lipofectamine™ MessengerMAX™ Transfection Reagent at a ratio of 0.5 μL per 1 μg of mRNA and incubated for 20 minutes. The resulting complex was added to the wells containing cultured LL/2 cells in an amount equivalent to 5 μg of mRNA, and the cells were incubated at 37° C. for 24 hours. At this time, DMEM medium was used.

After the incubation, 100 μL/well of lysis buffer and M-PER™ (Mammalian Protein Extraction Reagent) (Thermo Scientific™, Cat. No. 78501) were added to each well to lyse the cells and extract proteins. Protein quantification was performed using the Pierce™ BCA Protein Assay Kit (Thermo Scientific™, Cat. No. 23225). The obtained data were used to determine the amount of protein used in the experiment. The extracted samples were diluted with lysis buffer and 5× reducing dye (containing 10% SDS, 0.5% bromophenol blue, 50% glycerol, 0.5% 2-mercaptoethanol, and 250 mM Tris, pH 6.8) to equalize the protein concentrations.

Western blot analysis was performed on the diluted samples using an anti-RAS (G12D mutant) monoclonal antibody (CST, Cat. No. #14429S) and anti-β-actin antibody (Abcam, Cat. No. ab8227).

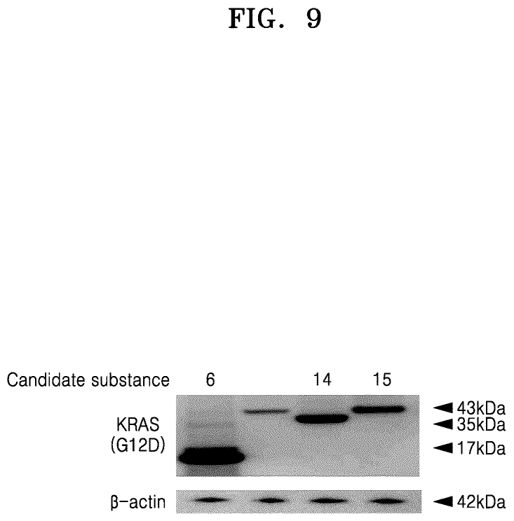

FIG. 9 shows the results of Western blotting using the anti-RAS (G12D mutant) monoclonal antibody for proteins expressed in LL/2 cells, a Lewis lung carcinoma cell line, into which mRNAs encoding KRAS variant peptide-linked Antigens 6, 14, and 15 were introduced.

As shown in FIG. 9, Antigens 6, 14, and 15, each encoding linked KRAS variant peptides, exhibited different molecular size and expression levels within the cells. When comparing expression levels with Antigen 6 as a control, Antigens 14 and 15 showed relatively higher expression, though still lower than that of Antigen 6.

These results suggest that the expression level of an antigen may vary depending on factors such as the amino acid sequence, composition, length of the KRAS variant peptides included in the antigen, and the overall sequence length. For example, among antigens containing the G12D variant peptide, the level of G12D protein expression may differ depending on sequence design, including the peptide's own sequence, its length, and the composition between peptides.

1-20. (canceled)

21. An antigenic peptide in which two or more KRAS variant peptides selected from the group consisting of KRAS variant peptides are linked, wherein the antigenic peptide comprises G12C, G12D, and G13D variant peptides.

22. The antigenic peptide of claim 21, wherein the variant peptide is an antigenic peptide having a length of 8, 17, or 31 amino acids (aa).

23. The antigenic peptide of claim 21, further comprising one or more of G12V, G12R, G13C, G13R, and Q61H variant peptides.

24. The antigenic peptide of claim 21, comprising G12C, G12D, G12V, G12R, G13C, G13D, and Q61H variant peptides, wherein each variant peptide has a length of 17 aa.

25. The antigenic peptide of claim 23, wherein the variant peptides have a length of 8 aa or 31 aa.

26. The antigenic peptide of claim 25, comprising linked variant peptides having identical mutant residues and having lengths of 8 aa and 31 aa.

27. The antigenic peptide of claim 26, comprising G12C, G12D, G12V, G12R, G13C, G13D, G13R and Q61H variant peptides.

28. The antigenic peptide of claim 21, wherein one or more of the variant peptides are linked to each other via a linker.

29. The antigenic peptide of claim 26, wherein the linker is G4S (SEQ ID NO: 552) or LLSVGG (SEQ ID NO: 553).

30. The antigenic peptide of claim 21, comprising the amino acid sequence of SEQ ID NO: 24, 28, 36, or 37.

31. An mRNA encoding the antigenic peptide of claim 1.

32. The mRNA of claim 11, comprising the nucleotide sequence of SEQ ID NO: 2, 6, 14, 15, 296, 297, 298, 299, or 300.

33. An immunogenic composition comprising the antigenic peptides of claim 21, or an mRNA encoding the antigenic peptide.

34. The immunogenic composition of claim 33, for preventing or treating cancer.

35. The immunogenic composition of claim 33, wherein the mRNA comprises a nucleotide sequence of SEQ ID NO: 2, 6, 14, 15, 296, 297, 298, 299, or 300.

36. The immunogenic composition of claim 35, wherein the mRNA is bound to a lipid nanoparticle.

37. The immunogenic composition of claim 34, wherein the cancer is pancreatic cancer or lung cancer.

38. A method of inducing an immune response to KRAS variant peptides in a subject, comprising administering to the subject the antigenic peptides of claim 21, or an mRNA encoding the antigenic peptide.

39. The method of claim 38, for preventing or treating cancer.

40. The method of claim 39, wherein the cancer is pancreatic cancer or lung cancer.
